SEQUENCE LISTING (14 sequences)

SEQ ID NO: 1 - DNA, 21 bp, Artificial Sequence
  Description of Artificial Sequence Synthetic primer
gggaatttggcgacgtaattc

SEQ ID NO: 2 - DNA, 18 bp, Artificial Sequence
  Description of Artificial Sequence Synthetic primer
gcggaggctgaggaacag

SEQ ID NO: 3 - DNA, 24 bp, Artificial Sequence
  Description of Artificial Sequence Synthetic primer
cctgtaccagtccaataccatcct

SEQ ID NO: 4 - DNA, 20 bp, Artificial Sequence
  Description of Artificial Sequence Synthetic primer
tcctgctggtccttcccata

SEQ ID NO: 5 - DNA, 19 bp, Artificial Sequence
  Description of Artificial Sequence Synthetic primer
aggacgcaaggagggtttg

SEQ ID NO: 6 - DNA, 20 bp, Artificial Sequence
  Description of Artificial Sequence Synthetic primer
gtccgccgagtccttactga

SEQ ID NO: 7 - DNA, 23 bp, Artificial Sequence
  Description of Artificial Sequence Synthetic primer
gaagcctattctgcaaagattgc

SEQ ID NO: 8 - DNA, 21 bp, Artificial Sequence
  Description of Artificial Sequence Synthetic primer
gagtaccccaatcgagccaaa

SEQ ID NO: 9 - DNA, 17 bp, Artificial Sequence
  Description of Artificial Sequence Synthetic primer
ggcctcggtgtgccttt

SEQ ID NO: 10 - DNA, 21 bp, Artificial Sequence
  Description of Artificial Sequence Synthetic primer
gatgtgcgcaatcatgtacgt

SEQ ID NO: 11 - DNA, 18 bp, Artificial Sequence
  Description of Artificial Sequence Synthetic primer
tgcgtctcttgccggaat

SEQ ID NO: 12 - DNA, 20 bp, Artificial Sequence
  Description of Artificial Sequence Synthetic primer
ggctcaccctccagaagctt

SEQ ID NO: 13 - DNA, 23 bp, Artificial Sequence
  Description of Artificial Sequence Synthetic primer
ctgaactggtgtatgcagattgc

SEQ ID NO: 14 - DNA, 17 bp, Artificial Sequence
  Description of Artificial Sequence Synthetic primer
ttccgagcggccaagtc

(19) United States
(12) Patent Application Publication
(10) Pub. No.: US 2009/0092979 A1
(43) Pub. Date: Apr. 9, 2009

(54) METHODS FOR ISOLATING LONG FRAGMENT RNA FROM FIXED SAMPLES

(75) Inventor: Kathleen Danenberg, Los Angeles, CA (US)

Correspondence Address: WOMBLE CARLYLE SANDRIDGE & RICE, PLLC, ATTN: PATENT DOCKETING 32ND FLOOR, P.O. BOX 7037, ATLANTA, GA, 30357-0037, US

(73) Assignee: Response Genetics, Inc., Los Angeles, CA (US)

(21) Appl. No.: 12/144,388

(22) Filed: Jun. 23, 2008

Related U.S. Application Data
(60) Provisional application No. 60/945,785, filed on Jun. 22, 2007.

(51) Int. Cl. C12Q 1/68; C07H 21/02

(52) U.S. Cl. 435/6; 536/25.41; 536/23.1

(57) ABSTRACT

The present invention relates to methods for the extraction of long fragment RNA from fixed tissue specimens. In particular, the present invention relates to methods for the extraction of RNA from formalin-fixed paraffin-embedded tissue specimens for use in biologic applications, including assays based on oligonucleotide hybridization.

This application claims priority to provisional application 60/945,785 filed on Jun. 22, 2007, which is herein incorporated by reference in its entirety.

### FIELD OF THE INVENTION

The present invention relates to the field of extraction and isolation of high yield and high quality (long fragment) RNA from fixed tissue samples. The present invention relates to the use of these novel extraction methods to provide a method for assessing gene expression levels for genes such as cancer biomarkers in fixed or fixed and paraffin embedded tissues. The present invention also provides a method for determining a chemotherapy based regimen by measuring mRNA levels of a certain biomaker in a patient's tumor cells and comparing it to a predetermined threshold expression levels.

### BACKGROUND

The quantitative measurement of RNA species is central to the pursuit of modern research in molecular biology. RNA species also has very important clinical significance, for example, in the preparation of gene expression profiles to characterize various different tissue types, such aggressive and non-aggressive tumors (1). Recent technological advances, including the development of highly sensitive fluorescence-based real-time RT-PCR procedures and other hybridization-dependent methodologies, now make it possible to perform rapid and specific quantification of very small amounts of mRNA, such as those obtained from patient biopsy specimens. Problems arise, especially in the application of RNA quantification to clinical studies, in obtaining RNA of sufficient quality for optimal results in most quantification methods.

Messenger RNA (mRNA) is reasonably stable in fresh/frozen tissue, and is also relatively easy to isolate in largely intact form. However, except for small studies in which special efforts are made to collect fresh-frozen tissue samples, biopsy tissue samples taken from patients are typically subjected to formalin fixation and embedding in paraffin. This is true of tissue specimens from patients routinely treated at hospitals as well as from participants in major clinical trials. The primary reason formalin fixation and paraffin embedding (FFPE) is the most commonly used method is to aid in the pathological examination of the tissue. Morphological examination of fresh-frozen tissue that has been cryostat-sectioned is suboptimal as it makes both molecular histopathological correlations difficult and purification of tumor or other tissues by micro-dissection more difficult. In contrast, the formalin-fixation and paraffin-embedding of tissue samples preserves the morphology and makes pathological examination much easier. The secondary reason FFPE is commonly used is the difficulty and expense of storing fresh-frozen tissue samples. The logistical problems involved in the acquisition and securing of sufficient tissue samples for diagnostic analysis and molecular assays seem insurmountable. The result is that few, if any, tissue banks worldwide contain enough frozen tissue samples suitable for a wide range of genetic analyses, or which have sufficiently long-term patient follow-up and outcome data. On the other hand, FFPE tissues remain the basis for current pathology practice. Archival FFPE tissues with long-term follow-up are readily available and easily accessible to both clinicians and researchers and as such represent an extensive source of genetic material for investigation in the clinical setting (2).

Unfortunately, the formalin fixation process, while better preserving tissue morphology, has adverse consequences for the RNA in the tissues. The RNA molecules become fragmented, that is cleaved into smaller pieces, as well as probably cross-linked by the formalin (3-6). Both of these processes greatly increase the difficulty of using RNA from FFPE specimens in RNA quantitation procedures such as the generation of gene expression profiles. Short length RNA makes it more difficult to obtain optimal primer-probe sets for quantitative real-time RT-PCR, whereas cross-linking prevents the advancement of the RNA or DNA polymerase enzymes that synthesize the new strands of RNA or DNA that are necessary to carry out successful amplification of the isolated RNA material. Using randomly-fragmented RNA from FFPE tissue also drastically reduces the yield of amplified RNA compared to that of fresh-frozen tissues, while short fragment length specifically decreases the efficiency and specificity of subsequent hybridization steps. Specificity of hybridization is critically important in avoiding false positive results and high background amplifications in PCR. For these reasons, it is important to develop methods to isolate the highest quality RNA possible in the highest yields possible from FFPE tissues.

Extraction of intact high molecular weight (long fragment) RNA from FFPE tissue has been a difficult and inconsistent process. Various techniques for extracting RNA from samples are known in the art (7-17). These extraction techniques have been tested with varying success. Some studies have provided new methods to optimize DNA and RNA extraction from archival FFPE tissues (18-34), whereas other studies have investigated the effect of duration of fixation on quantitative RT-PCR analyses (a factor over which researchers generally have no control) (10,12,13,26). The studies to date have shown that while it is possible to extract RNA from FFPE that can be successfully subjected to PCR, there are still problems with consistency of isolation yield and quality (length) of the extracted RNA. Previous attempts to amplify fragments of extracted RNA longer than 200 nucleotides (nt) were usually unsuccessful and only amplification of fragments in a range of 60 to 120 nt was generally achieved with any degree of success (2). The emphasis of most studies of extraction methods to date has focused on obtaining the maximal yield of RNA from FFPE tissue, not necessarily discovering how to obtain high quality extracted RNA, that is, preserving the fragment length of RNA by avoiding further degradation during the extraction process. These two goals are not always compatible: obtaining the maximal yield of RNA may require conditions that further degrade the length of the RNA, resulting in more RNA but of shorter lengths.

Another factor that is less often recognized and not generally addressed in most previous studies is DNA contamination of the RNA preparations. Because DNA is a more stable molecule than RNA, FFPE extractions often contain more DNA than RNA. RNA and DNA are chemically very similar molecules and the base sequences of DNA are replicated in the RNA. Thus, in RNA analysis entailing hybridization technologies, a large amount of DNA contamination can lead to spurious results because DNA may compete with the RNA molecules for binding to the hybridization sites. For example, during the PCR, primers and probes can bind to and amplify the contaminating DNA as well as the cDNA that has been generated from the RNA. The presence of DNA in RNA isolations for RT-PCR is often dealt with by using so-called RNA specific primers, that is, primers that cross intron-exon junctions and thus should not amplify corresponding gene sequences in DNA. Even with RNA specific primers, however, pseudo-genes in the DNA can be amplified. One benefit of a lack of appreciable DNA in the sample preparation is that one is not restricted to RNA-specific primers in carrying out RT-PCR and, thus, the choice of primer binding sites is greatly increased.

Accordingly there is a need for a method of isolating high quality (long fragment) RNA in high yields with acceptably low DNA co-isolation/contamination. The present invention satisfies this need.

Isolating RNA to determine expression levels of various biomakers in cancer tissues can be useful in diagnosis of certain conditions or in assisting a physician in determining a proper course of therapy. For example, biomakers have been identified that are useful in diagnosing cancer as well as useful in predicting whether a certain chemotherapeutic regimen would be helpful in treating the disease. Many disease biomarkers are known and include, for example cancer biomarkers ERCC1, TS, DPD, Her2-neu, EGFR, GST-pi, k-ras and RRM1 to name a few.

In addition, the methods of RNA isolation disclosed herein can be used to isolate long fragment RNA from any FFPE tissue. Normally, the FFPE tissue will be from a tumor biopsy from any cancer.

### ERCC1

The excision repair cross-complementing (ERCC1) gene is essential in the repair of DNA adducts. The human ERCC1 gene has been cloned. Westerveld et al., Nature (London) 310:425 428 (1984); Tanaka et al., Nature 348:73 76 (1990). Several studies using mutant human and hamster cell lines that are defective in this gene and studies in human tumor tissues indicate that the product encoded by ERCC1 is involved in the excision repair of platinum-DNA adducts. Dabholkar et al., J. Natl. Cancer Inst. 84:1512 1517 (1992); Dijt et al., Cancer Res. 48:6058 6062 (1988); Hansson et al., Nucleic Acids Res. 18: 35 40 (1990).

When transfected into DNA-repair deficient CHO cells, ERCC1 confers cellular resistance to cisplatin along with the ability to repair platinum-DNA adducts. Hansson et al., Nucleic Acids Res. 18: 35 40 (1990). Currently accepted models of excision repair suggest that the damage recognition/excision step is rate-limiting to the excision repair process.

The relative levels of expression of excision repair genes such as ERCC1 in malignant cells from cancer patients receiving platinum-based therapy has been examined. Dabholkar et al., J. Natl. Cancer Inst. 84:1512 1517 (1992). ERCC1 over-expression in gastric cancer patients has been reported to have a negative impact on tumor response and ultimate survival when treated with the chemotherapeutic regimen of cisplatin (DDP)/fluorouracil (Metzger, et al., J Clin Oncol 16: 309, 1998). Recent evidence indicates that gemcitabine (Gem) may modulate ERCC1 nucleotide excision repair (NER) activity. Thus, intratumoral levels of ERCC1 expression may be a major prognostic factor for determining whether or not DDP and GEM would be an effective therapeutic cancer patients.

### GST-pi

The glutathione-S-transferase (GST) family of proteins is involved in detoxification of cytotoxic drugs. By catalyzing the conjugation of toxic and carcinogenic electrophilic molecules with glutathione the GST enzymes protect cellular macromolecules from damage (Boyer et al., Preparation, characterization and properties of glutathione S-transferases. In: Zakim D, Vessey D (eds.) Biochemical Pharmacology and Toxicology. New York, N.Y.: John Wiley and Sons, 1985.). A certain isomeric type of these proteins, the glutathione S-transferase Pi (GST-pi, also to be interchangeably referred to as GSTP1 or GST-π herein) is widely expressed in human epithelial tissues and has been demonstrated to be over-expressed in several tumors (Terrier et al., Am J Pathol 1990; 137: 845 853; Moscow et al., Cancer Res 1989; 49: 1422 1428). Increased GST-pi levels have been found in drug resistant tumors, although the exact mechanism remains unclear (Tsuchida et al., Crit. Rev Biochem Mol Biol 1992; 27: 337 384). Previous studies have suggested that low expression of GST protein (not mRNA) is associated with response to platinum-based chemotherapy (Nishimura et al., Cancer. Clin Cancer Res 1996; 2:1859 1865; Tominaga, et al., Am. J. Gastro. 94:1664 1668, 1999; Kase, et al., Acta Cytologia. 42: 1397 1402, 1998). However, these studies did not measure quantitative gene expression, but used a semi-quantitative immunohistochemical staining method to measure protein levels. However, quantitative GST-pi gene expression measurements are needed to achieve a very effective prognostication.

Her2 neu/EGFR

Lung cancer is the leading cause of cancer-related deaths among both males and females in western countries. In the United States, approximately 171,000 new cases of lung cancer are diagnosed and 160,000 individuals die from this disease each year. Despite improvements in the detection and treatment of lung cancer in the past two decades, the overall 5-year survival remains less than 15%. Ginsberg, et al., In: DeVita, et al., Cancer: Principles in Practice of Oncology, Ed. 5, pp. 858-910. Philadelphia Lipincott-Raven Publishers, 1997. To further improve the survival rate in patients with Non-Small Cell Lung Carcinoma (NSCLC), their prognostic classification based on molecular alterations is crucial. Such classification will provide more accurate and useful diagnostic tools and, eventually, more effective therapeutic options.

Receptor tyrosine kinases (RTKs) are important in the transduction of mitogenic signals. RTKs are large membrane spanning proteins that possess an extracellular ligand binding domain for growth factors such as epidermal growth factor (EGF) and an intracellular portion that functions as a kinase to phosphorylate tyrosine amino acid residues on cytosol proteins thereby mediating cell proliferation. Various classes of receptor tyrosine kinases are known based on families of growth factors that bind to different receptor tyrosine kinases. (Wilks, Advances in Cancer Research, 1993, 60, 43-73).

Class I kinases such as the EGF-R family of receptor tyrosine kinases include the EGF, HER2-neu, erbB, Xmrk, DER and let23 receptors. These receptors are frequently present in common human cancers such as breast cancer (Sainsbury et al., Brit. J. Cancer, 1988, 58, 458; Guerin et al., Oncogene Res., 1988, 3, 21), squamous cell cancer of the lung (Hendler et al., Cancer Cells, 1989, 7, 347), bladder cancer (Neal et al., Lancet, 1985, 366), oesophageal cancer (Mukaida et al, Cancer, 1991, 68, 142), gastrointestinal cancer such as colon, rectal or stomach cancer (Bolen et al., Oncogene Res., 1987, 1, 149), leukemia (Konaka et al., Cell, 1984, 37, 1035) and ovarian, bronchial or pancreatic cancer (European Patent Specification No. 0400586). As further human tumor tissues are tested for the EGF family of receptor tyrosine kinases it is expected that its widespread prevalence will be established in other cancers such as thyroid and uterine cancer.

Specifically, EGFR tyrosine kinase activity is rarely detected in normal cells whereas it is more frequently detectable in malignant cells (Hunter, Cell, 1987, 50, 823). It has been more recently shown that EGFR is over expressed in many human cancers such as brain, lung squamous cell, bladder, gastric, breast, head and neck, oesophageal, gynecological and thyroid tumors. (W J Gullick, Brit. Med. Bull., 1991, 47, 87). Receptor tyrosine kinases are also important in other cell-proliferation diseases such as psoriasis. EGFR disorders are those characterized by EGFR expression by cells normally not expressing EGFR, or increased EGFR activation leading to unwanted cell proliferation, and/or the existence of inappropriate EGFR levels. The EGFR is known to be activated by its ligand EGF as well as transforming growth factor-alpha (TGF-α).

The Her2-neu protein is also a member of the class I receptor tyrosine kinase (RTK) family. Yarden and Ullrich, Annu. Rev. Biochem. 57:443, 1988; Ullrich and Schlessinger, Cell 61:203, 1990. Her2-neu protein is structurally related to EGFR. Carraway, et al., Cell 78:5, 1994; Carraway, et al., J. Biol. Chem. 269:14303, 1994. These receptors share a common molecular architecture and contain two cysteine-rich regions within their cytoplasmic domains and structurally related enzymatic regions within their cytoplasmic domains.

Ligand-dependent activation of Her2-neu protein is thought to be mediated by neuactivating factor (NAF), which can directly bind to p165(Her2-neu) and stimulate enzymatic activity. Dougall et al., Oncogene 9:2109, 1994; Samata et al., Proc. Natl. Acad. Sci. USA 91:1711, 1994. Ligand-independent homodimerization of Her2-neu protein and resulting receptor activation is facilitated by over-expression of Her2-neu protein. An activated Her2-neu complex acts as a phosphokinase and phosphorylates different cytoplasmic proteins. HER2-neu disorders are characterized by inappropriate activity or over-activity of HER2-neu have increased HER2-neu expression leading to unwanted cell proliferation such as cancer.

Inhibitors of receptor tyrosine kinases EGFR and HER2-neu are employed as selective inhibitors of the growth of mammalian cancer cells (Yaish et al. Science, 1988, 242, 933). For example, erbstatin, an EGF receptor tyrosine kinase inhibitor, reduced the growth of EGFR expressing human mammary carcinoma cells injected into athymic nude mice, yet had no effect on the growth of tumors not expressing EGFR. (Toi et al., Eur. J. Cancer Clin. Oncol., 1990, 26, 722.) Various derivatives of styrene are also stated to possess tyrosine kinase inhibitory properties (European Patent Application Nos. 0211363, 0304493 and 0322738) and to be of use as anti-tumour agents. Two such styrene derivatives are Class I RTK inhibitors whose effectiveness has been demonstrated by attenuating the growth of human squamous cell carcinoma injected into nude mice (Yoneda et al., Cancer Research, 1991, 51, 4430). It is also known from European Patent Applications Nos. 0520722 and 0566226 that certain 4-anilinoquinazoline derivatives are useful as inhibitors of receptor tyrosine kinases. The very tight structure-activity relationships shown by these compounds suggests a clearly-defined binding mode, where the quinazoline ring binds in the adenine pocket and the anilino ring binds in an adjacent, unique lipophilic pocket. Three 4-anilinoquinazoline analogues (two reversible and one irreversible inhibitor) have been evaluated clinically as anticancer drugs. Denny, Farmaco January-February 2001; 56(1-2):51-6. Recently, the U.S. FDA approved the use of the monoclonal antibody trastazumab (Herceptin®) for the treatment of HER2-neu over expressing metastatic breast cancers. Scheurle, et al., Anticancer Res 20:2091-2096, 2000.

Because effective chemotherapy against tumors often requires a combination of agents, the identification and quantification of determinants of resistance or sensitivity to each single drug has become an important tool to design individual combination chemotherapy. Studies have unsuccessfully attempted to reliably correlate the relative levels of expression of EGFR and/or HER2-neu in malignant cells from cancer patients with survivability.

The prognostic importance of EGFR and in NSCLC has heretofore remained controversial. Studies using binding assays correlated increased EGFR expression with advanced stage NSCLC and shortened overall survival, whereas studies using semi-quantitative techniques for measuring EGFR mRNA or protein expression failed to show a consistent correlation with clinical outcome. Veale et al., Br. J. Caner 68:162-165, 1993; Fujino et al., Eur. Cancer 32:2070-2074, 1996; Rusch, et al., Cancer Res 53:2379-2385, 1993; Pfeiffer, et al., Br J Cancer 74:86-91, 1996; Pastorino, et al.,. J Clin Oncol 15:2858-2865, 1997. Studies of EGFR expression in NSCLC tumors using immunohistochemical methods have shown frequencies for EGFR over expression between 32% and 47% in NSCLC tumors. Veale et al., Br. J. Caner 55:513-516, 1987; Veale et al., Br. J. Caner 68:162-165, 1993; Fujino et al., Eur. Cancer 32:2070-2074, 1996; Rusch, et al., Cancer Res 53:2379-2385, 1993; Pastorino et al., J. Clin. One. 15:2858-2865, 1997; Tateishi, et al., Eur J Cancer 27:1372-75, 1991; Rachwal, et al., Br J Cancer 72:56-64, 1995; Rusch, et al., Cancer Res 15:2379-85, 1993; Pfeiffer, et al., Br J Cancer 78:96-9, 1998; Ohsaki, et al., Oncol Rep 7:603-7, 2000. Moreover, significant differences in EGFR expression has been reported among histological subtypes, generally with higher EGFR expression in SCC compared to AC and LC. Fujino et al., Eur. Cancer 32:2070-2074, 1996; Veale et al., Br. J. Caner 55:513-516, 1987; Pastorino et al., J. Clin. One. 15:2858-2865, 1997; Pfeiffer, et al., Br J Cancer 78:96-9, 1998; Ohsaki et al., Oncol. Rep. &:603-7, 2000. However, these studies reported no consistent correlation of EGFR over expression with lung cancer patient survival.

Observations of a purported correlation of EGFR over expression with a decrease in patient survival were made in some inconclusive studies. Veale et al., 1987; Ohsaki et al., 2000. However, Veale et al., analyzed a population of only nineteen NSCLC patients. Ohsaki et al., correlated EGFR protein expression with poor prognosis in NSCLC patients with p53 over expression (P=0.024).

As with EGFR, the prognostic importance of HER2-neu and in NSCLC has heretofore remained controversial. HER2-neu protein over expression has been demonstrated in NSCLC, including squamous cell carcinoma, adenocarcinoma, and large cell carcinoma. Veale et al., 1987; Schneider, et al., Cancer Res 49:4968-4971, 1989; Kern et al., Cancer Res. 50:5184-5191, 1990; Weiner, et al., Cancer Res 50:421425, 1990; Scheurle, et al., Anticancer Res. 20:2091-2096, 2000. Earlier studies, using protein assays, reported an association of HER2-neu protein over expression and inferior overall survival in pulmonary adenocarcinomas (AC). Kern, et al., Cancer Res 50:5184-5191, 1990; Kern et al., J Clin Invest 93:516-20, 1994. However, contradictory studies reported no correlation of HER2-neu protein over expression with inferior overall survival in pulmonary adenocarcinomas (AC). Pfeiffer et al., Br. J. Cancer 74:86-91, 1996.

Another critical question is the evaluation of interrelationships between HER2-neu and EGFR co-over expression as prognosticators of cancer. Tateishi et al., (Eur. J. Cancer 27:1372-75, 1991), measured EGFR and HER2-neu protein co-expression, in 13% of AC analyzed, and found that co-over expression of these two genes correlated with inferior five-year survival. However, as with HER2-neu over expression alone, association between HER2-neu and EGFR co-expression and survival in squamous cell carcinoma (SCC) and large cell carcinoma (LCC) of the lung has not been reported.

Inconsistent methodologies for the determination of EGFR and HER2-neu expression levels has been at the root of the problem in determining to what extent expression of these genes may be used to prognosticate cancer patient survivability. Heretofore investigations of HER2-neu and EGFR expression in NSCLC has resulted in enormous variations in frequencies of NSCLC tumors scored positive for both EGFR and HER2-neu expression. Over expression of HER2-neu, defined as positive protein staining in adenocarcinomas (AC), was reported in 13-80%, in 2-45% in squamous cell carcinomas (SCC), and in 0-20% in large cell carcinomas (LC) by using paraffin embedded tissue on light microscope slides and HER2-neu antisera. Pfeiffer et al., 1996; Kern et al., 1990; Kern et al., 1994; Tateishi et al., 1991; Shi, et al., Mol Carcing 5:213-8, 1992; Bongiorno, et al., J Thorac Cardiovasc Surg 107:590-5, 1994; Harpole, et al., Clin Cancer Res 1:659-64, 1995; Volm et al., Anticancer Res 12:11-20, 1992. Moreover, a recent report illustrates the non-specificity of current protocols designed to assess HER2-neu expression levels. The Hercep Test® for measurement of HER2-neu expression in invasive breast cancers was shown to have very high false positivity. Jacobs et al., J Clin Oncol 17:1983-1987, 1999.

If a precise, accurate, and consistent method for determining the expression levels of EGFR and HER2-neu existed, one could ascertain what expression levels correlate to patient survivability and whether or not a receptor tyrosine kinase targeted chemotherapy is appropriate. Consistent demonstration of EGFR and/or HER2-neu over expression in NSCLC, using a standardized method, is desirable in establishing clinical trials for current and future receptor tyrosine kinase targeted chemotherapies, e.g., chemotherapeutic agents, antibody-based drugs, to treat cancers over expressing these receptors.

### DPD

5-Fluorouracil (5-FU) is a very widely used drug for the treatment of many different types of cancers, including major cancers such as those of the GI tract and breast (Moertel, C. G. New Engl. J. Med., 330:1136-1142, 1994). For more than 40 years the standard first-line treatment for colorectal cancer was the use of 5-FU alone, but it was supplanted as “standard of care” by the combination of 5-FU and CPT-11 (Saltz et al., Irinotecan Study Group. New England Journal of Medicine. 343:905-14, 2000). Recently, the combination of 5-FU and oxaliplatin has produced high response rates in colorectal cancers (Raymond et al, Semin. Oncol., 25:4-12, 1998). Thus, it is likely that 5-FU will be used in cancer treatment for many years because it remains the central component of current chemotherapeutic regimens. In addition, single agent 5-FU therapy continues to be used for patients in whom combination therapy with CPT-11 or oxaliplatin is likely to be excessively toxic.

5-FU is typical of most anti-cancer drugs in that only a minority of patients experience a favorable response to the therapy. Large randomized clinical trials have shown the overall response rates of tumors to 5-FU as a single agent for patients with metastatic colorectal cancer to be in the 15-20% range (Moertel, C. G. New Engl. J. Med., 330:1136-1142, 1994). In combination with other chemotherapeutics mentioned above, tumor response rates to 5-FU-based regimens have been increased to almost 40%. Nevertheless, the majority of treated patients derive no tangible benefit from having received 5-FU based chemotherapy, and are subjected to significant risk, discomfort, and expense. Since there has been no reliable means of anticipating the responsiveness of an individual's tumor prior to treatment, the standard clinical practice has been to subject all patients to 5-FU-based treatments, fully recognizing that the majority will suffer an unsatisfactory outcome.

The mechanism of action and the metabolic pathway of 5-FU have been intensively studied over the years to identify the most important biochemical determinants of the drug's anti-tumor activity. The ultimate goal was to improve the clinical efficacy of 5-FU by: a) modulation of its intracellular metabolism and biochemistry; and b) measuring response determinants in patients' tumors prior to therapy to predict which patients are most likely to respond (or not to respond) to the drug. Two major determinants emerged from these studies: 1) the identity of the target enzyme of 5-FU, thymidylate synthase (TS) and 2) the identity of the 5-FU catabolic enzyme, dihydropyrimidine dehydrogenase (DPD).

The first studies in the area of tumor response prediction to 5-FU based therapy centered on the target enzyme TS in colorectal cancer. Leichman et al (Leichman et al., J. Clin Oncol., 15:3223-3229, 1997) carried out a prospective clinical trial to correlate tumor response to 5-FU with TS gene expression as determined by RT-PCR in pre-treatment biopsies from colorectal cancers. This study showed: 1) a large 50-fold range of TS gene expression levels among these tumors; and 2) strikingly different levels of TS gene expression between responding and non-responding tumors. The range of TS levels of the responding groups (0.5−4.1×10⁻³, relative to an internal control) was narrower than that of the non-responding groups (1.6−23.0×10⁻³, relative to an internal control). The investigators determined a resulting “non-response cutoff” threshold level of TS expression above which there were only non-responders. Thus, patients with TS expression above this “non-response cutoff” threshold could be positively identified as non-responders prior to therapy. The “no response” classification included all therapeutic responses with <50% tumor shrinkage, progressing growth resulting in a >25% tumor increase and non-progressing tumors with either <50% shrinkage, no change or <25% increase. These tumors had the highest TS levels. Thus, high TS expression identifies particularly resistant tumors. TS expression levels above a certain threshold identified a subset of tumors not responding to 5-FU, whereas TS expression levels below this number predicted an appreciably higher response rate yet did not specifically identify responding tumors.

Subsequent studies investigated the usefulness of DPD expression levels as a tumor response determinant to 5-FU treatment in conjunction with TS expression levels. DPD is a catabolic enzyme that reduces the 5,6 double bond of 5-FU, rendering it inactive as a cytotoxic agent. Previous studies have shown that DPD levels in normal tissues could influence the bio-availability of 5-FU, thereby modulating its pharmacokinetics and anti-tumor activity (Harris et al, Cancer Res., 50: 197-201, 1990). Additionally, evidence has been presented that DPD levels in tumors are associated with sensitivity to 5-FU (Etienne et al, J. Clin. Oncol., 13: 1663-1670, 1995; Beck et al., Eur. J. Cancer, 30: 1517-1522, 1994). Salonga et al, (Clin Cancer Res., 6:1322-1327, 2000) investigated gene expression of DPD as a tumor response determinant for 5-FU/leucovorin treatment in a set of tumors in which TS expression had already been determined. As with TS, the range of DPD expression among the responding tumors was relatively narrow (0.6−2.5×10⁻³, 4.2-fold; relative to an internal control) compared with that of the non-responding tumors (0.2−16×10⁻³, 80-fold; relative to an internal control). There were no responding tumors with a DPD expression greater than a threshold level of about 2.5×10⁻³. Furthermore, DPD and TS expression levels showed no correlation with one another, indicating that they are independently regulated genes. Among the group of tumors having both TS and DPD expression levels below their respective “non-response cut-off” threshold levels, 92% responded to 5-FU/LV. Thus, responding tumors could be identified on the basis of low expression levels of DPD and TS.

DPD is also an important marker for 5-FU toxicity. It was observed that patients with very low DPD levels (such as in DPD Deficiency Syndrome; i.e. thymine uraciluria) undergoing 5-FU based therapy suffered from life-threatening toxicity (Lyss et al., Cancer Invest., 11: 2390240, 1993). Indeed, the importance of DPD levels in 5-FU therapy was dramatically illustrated by the occurrence of 19 deaths in Japan from an unfavorable drug interaction between 5-FU and an anti-viral compound, Sorivudine (Diasio et al., Br. J. Clin. Pharmacol. 46, 1-4, 1998). It was subsequently discovered that a metabolite of Sorivudine is a potent inhibitor of DPD. This treatment resulted in DPD Deficiency Syndrome-like depressed levels of DPD which increased the toxicity of 5-FU to the patients (Diasio et al., Br. J. Clin. Pharmacol. 46, 1-4, 1998).

Thus, because of: a) the widespread use of 5-FU protocols in cancer treatment; b) the important role of DPD expression in predicting tumor response to 5-FU; and c) the sensitivity of individuals with DPD-Deficiency Syndrome to common 5-FU based treatments, it is clear that accurate determination of DPD expression levels prior to chemotherapy will provide an important benefit to cancer patients.

Measuring DPD enzyme activity requires a significant amount of fresh tissue that contains active enzyme. Unfortunately, most pre-treatment tumor biopsies are available only as fixed paraffin embedded (FPE) tissues, particularly formalin-fixed paraffin embedded tissues which do not contain active enzyme. Moreover, biopsies generally contain only a very small amount of heterogeneous tissue.

RT-PCR primer and probe sequences are available to analyze DPD expression in frozen tissue or fresh tissue. However, those primers are unsuitable for the quantification of DPD mRNA from fixed tissue by RT-PCR. Heretofore, existing primers give no or erratic results. This is thought to be due to the: a) inherently low levels of DPD RNA; b) very small amount of tissue embedded in the paraffin; and c) degradation of RNA in the paraffin into short pieces of <100 bp. As a result, other investigators have made a concerted, yet unsuccessful efforts to obtain oligonucleotide primer sets allowing for such a quantification of DPD expression in paraffinized tissue. Thus, there is a need for method of quantifying DPD mRNA from fixed tissue to provide an early prognosis for proposed cancer therapies. Because it has been shown that DPD enzyme activity and corresponding mRNA expression levels are well correlated (Ishikawa et al., Clin. Cancer Res., 5:883-889, 1999; Johnson et al, Analyt. Biochem. 278: 175-184, 2000), measuring DPD mRNA expression in FPE specimens provides a way to assess the DPD expression levels status of patients without having to determine enzyme activity in fresh tissues. Furthermore, FPE specimens are readily amenable to microdissection, so that DPD gene expression can be determined in tumor tissue uncontaminated with stromal tissue.

### TS

Thymidylate synthase (TS) is an integral enzyme in DNA biosynthesis where it catalyzes the reductive methylation of deoxyuridine monophosphate (dUMP) to deoxythymidine monophosphate (dTMP) and provides the only route for de novo synthesis of pyrimidine nucleotides within the cell (Johnston et al., 1995). Thymidylate synthase is a target for chemotherapeutic drugs, most commonly the antifolate agent 5-fluorouracil (5-FU). As the most effective single agent for the treatment of colon, head and neck and breast cancers, the primary action of 5-FU is to inhibit TS activity, resulting in depletion of intracellular thymine levels and subsequently leading to cell death.

Considerable variation in TS expression has been reported among clinical tumor specimens from both primary tumors (Johnston et al., 1995; Lenz et al., 1995) and metastases (Farrugia et al., 1997; Leichmann et al., 1997). In colorectal cancer, for example, the ratio of TS expression in tumor tissue relative to normal gastrointestinal mucosal tissue has ranged from 2 to 10 (Ardalan and Zang, 1996).

Thymidylate synthase is also known to have clinical importance in the development of tumor resistance, as demonstrated by studies that have shown acute induction of TS protein and an increase in TS enzyme levels in neoplastic cells after exposure to 5-FU (Spears et al. 1982; Swain et al. 1989). The ability of a tumor to acutely overexpress TS in response to cytotoxic agents such as 5-FU may play a role in the development of fluorouracil resistance. Previous studies have shown that the levels of TS protein directly correlate with the effectiveness of 5-FU therapy, that there is a direct correlation between protein and RNA expression (Jackman et al., 1985) and that TS expression is a powerful prognostic marker in colorectal and breast cancer (Jackman et al., 1985; Horikoshi et al., 1992).

In advanced metastatic disease, both high TS mRNA, quantified by RT-PCR, and high TS protein expression, have been shown to predict a poor response to fluoropyrimidine-based therapy for colorectal (Johnston et al., 1995, Farrugia et al., 1997, Leichman et al., 1997), gastric (Lenz et al., 1995, Alexander et al., 1995), and head and neck (Johnston et al., 1997) cancers. A considerable overlap between responders and non-responders was often present in the low TS category, but patients with TS levels above the median were predominantly nonresponders. The predictive value of TS over expression may be further enhanced if combined with other molecular characteristics such as levels of dihydropyrimidine dehydrogenase (DPD) and thymidine phosphorylase (TP) expression, replication error positive (RER+) status (Kitchens and Berger 1997), and p53 status (Lenz et al., 1997). Studies to date that have evaluated the expression of TS in human tumors suggest that the ability to predict response and outcome based upon TS expression in human tumors may provide the opportunity in the future to select patients most likely to benefit from TS-directed therapy.

### SUMMARY OF THE INVENTION

One aspect of the present invention is to provide a method for the extraction of RNA from fixed tissue specimens. The invention also provides reliable and reproducible methods for the isolation of RNA from formalin-fixed paraffin-embedded tissues.

The present invention provides a method for the isolation of long fragment RNA from fixed tissue samples comprising heating the fixed tissue sample in an extraction solution to a temperature in the range of about 44 to about 62° C. for a time period of 3 hours or more, wherein the extraction solution comprises a chelator at a concentration of about 0.1 mM to about 20 mM, and proteinase K (preferably at a concentration of about 5 μg proteinase K/400 μL, (12.5 μg proteinase K/mL)); and removing DNA contamination and isolating said RNA from the extraction solution.

In certain embodiments the heating may be at a temperature ranging from about 45 to about 60° C., from about 48 to about 58° C., from about 48 to about 55° C., from about 48 to 52° C., or about 50° C. An especially preferred heating temperature is from about 50-56° C.

In certain embodiments, the time period is greater than 4 hours, greater than 8 hours, greater than 12 hours, greater than 14 hours or about 16 hours. In a preferred embodiment the time period is about 16 hours.

The chelator may be any chelator such as, EDTA, EGTA, citrates, citric acids, salicylic acid, salts of salicylic acids, phthalic acids, 2,4-pentanedines, histidines, histidinol dihydrochlorides, 8-hydroxyquinolines, 8-hydroxyquinoline, citrates or o-hydroxyquinones. In a preferred embodiment, the chelator is EDTA or sodium citrate.

In certain embodiments, the chelator is EDTA or sodium citrate and is present at a concentration of about 2.5 mM to about 5.0 mM. In certain embodiments, the EDTA or sodium citrate is present at a concentration of about 2.5 mM to about 5.0 mM, at a concentration of about 3.0 mM to about 4.0 mM, at a concentration of about 3.25 to about 3.75 mM. In a preferred embodiment, the EDTA or sodium citrate is present at a concentration of about 0.6 mM to about 3.6 mM. In another preferred embodiment, EDTA or sodium citrate is present at about 3.6 mM.

DNA contamination can be removed by methods known in the art, such as but not limited to a phenol/chloroform/isoamyl (PCI) alcohol extraction, by two phenol/chloroform/isoamyl (both with or without the addition of DNAse) in the presence of a chaotropic agent in either the first PCI extraction, the second PCI extraction or both, or commercially available purification columns (i.e. Qiagen with or without DNase, or other products) (i.e. Ambion Turbo DNase free process). In some embodiments, a mixture of these methods may be employed.

The chaotropic agent may be any known chaotrope such as urea, guanidinium isothiocyanate, sodium thiocyanate (NaSCN), Guanidine HCl, guanidinium chloride, guanidinium thiocyanate, lithium tetrachloroacetate, sodium perchlorate, rubidium tetrachloroacetate, potassium iodide or cesium trifluoroacetate. In certain embodiments, chaotropic agent is guanidinium isothiocyanate.

In certain embodiments, the fixed formalin-fixed paraffin embedded tissue sample is 16 years old or younger. In a preferred embodiment, the tissue sample is 5 years old or younger and in preferred embodiments, the tissue sample is 2 years old or younger.

In certain embodiments, the long fragment RNA is longer than 200 nucleotides in length. In other embodiments, the long fragment RNA is 300 nucleotides or longer. In a preferred embodiment, the long fragment RNA is between about 300 to about 400 nucleotides in length.

### BRIEF DESCRIPTION OF THE FIGURES

FIGS. 1A and 1B show the effects of temperature on the yield of long fragment RNA. These data show that longer incubation times at lower temperatures would isolate a higher yield of longer fragment RNA.

FIGS. 2 A and B show the effect of heating time on the yield of RNA. The data show that the yield of all sizes of RNA fragments increased at longer heating times. The yield of 100 bp fragments increased by over 10 fold (3.5 PCR cycles) while that of 300 bp fragments increased by almost 2⁶ or about 600-fold when the heating times were increased. The yield of 400 bp fragments similarly increased.

FIGS. 3A and B show the effect of incubation temperature and EDTA concentration of the extraction solution on RNA from FFPE tissue. These data suggest 50° C. as a preferred heating temperature and 3.6 mM as a preferred concentration of EDTA.

FIG. 4 shows the effect of varying the amount of proteinase K in the extraction procedure to remove RNA from the paraffin matrix. The data show that 5 μg (1× in the figure) is a preferred concentration for both maximal RNA yield as well as minimal DNA contamination.

FIGS. 5A and 5B show the results of a comparison of RNA yield and DNA contamination using five extraction methods: 1) high temperature chaotrope method; 2) a PK method (a method of the present invention comprising (proteinase K, low temperature, and long heating time); 3) the PK method using a single phenol extraction with guanidine isothiocyanate (“GITC”); 4) the PK method using a double phenol extraction with GITC in the second extraction; 5) the PK method using a double phenol extraction with Tris buffer instead of GITC. For example, in the figure, the designation of “F4_{—}1_{—}100 bp” means sample F4 treated with the high temperature chaotrope method; F4_{—}2_{—}100 bp means sample F4 treated with the PK method; F4_{—}3_{—}100 bp means sample F4 treated with the PK method using a single phenol extraction with GITC, and so on. The last 5 bars on the left of the chart are designated as NRT (no reverse transcription) and indicate the amount of DNA in the samples.

FIG. 6 shows a comparison of the amount and purity of RNA isolated from FFPE samples designated as B5, D6 and F5. “PK” indicates the use of an isolation method of the present invention, (“RGI”) indicates the use of high temperature isolation method (set forth in U.S. Pat. No. 6,248,535); and “para” indicates the use of the commercially-available Paradise® kit. The purity of the RNA is measured by ultraviolet absorbance at 280 nm. These data show that the present invention isolates a higher yield of RNA and a more pure RNA (as opposed to DNA contaminated) than the Paradise® kit in the 3 samples tested. The results also show that the PK method does not produce as much RNA as the RGI method, but provides a more pure RNA sample.

FIG. 7 shows a comparison of the amounts of the 100, 300, 400 and 1000 bp RNA fragments isolated from FFPE samples B5, D6 and F5 as measured by PCR amplification of β-actin in each sample. “PK” indicates the use of an isolation method of the present invention, (“RGI”) indicates the use of high temperature isolation method (set forth in U.S. Pat. No. 6,248,535); and “para” indicates the use of the commercially-available Paradise® kit. The data show that the preferred method using PK gives the optimal yield of each fragment length as well as the least DNA contamination. The box below the bar graph provides the numerical data represented in the bar graph.

FIG. 8 shows a comparison of the size distributions of RNA fragments isolated by each method. The RNA is fractionated on a size-exclusion column and quantitated by UV absorbance at 280 nm. Smaller fragments migrate through the column faster than larger ones. D5=sample 1; F5=sample 2; D6=sample 3. “PK” indicates the use of an isolation method of the present invention, (“RGI”) indicates the use of high temperature isolation method (set forth in U.S. Pat. No. 6,248,535); “Paradise” indicates the use of the commercially-available Paradise® kit; and “Frozen” is a fractionation of RNA isolated from the corresponding fresh-frozen tissue. The single plot in the fifth row contains molecular weight standards. This figure shows that the PK method provides better quality longer fragment RNA than the other methods.

FIG. 9 shows a comparison of the level of β-actin expression (as determined by the PCR) in RNA isolated from FFPE tissue using the present invention to RNA isolated from fresh frozen tissue using conventional methods. These data show that analysis of gene expression obtained with RNA extracted from FFPE correlates, especially using a method of the present invention, and reliably reflects gene expression in fresh-frozen tissue. For example, a direct correlation between the result of fresh-frozen tissue with that of FFPE would show a slope and R² value as 1. The closer the R² value is to one, the closer the quality. Thus, for the PK isolation, the R² value is 0.89, which is better than the Paradise kit obtained R² value of 0.81 or the RGI R² value of 0.84.

FIG. 10 illustrates the effect of sample age on the extraction yield of long fragment RNA species. The data shows a progressive increase in Ct values (lower yield of RNA) with sample age. Thus, as the samples age, the yield and quality of RNA goes down (less long fragment RNA). Samples 1, 2, 3, 4 and 5 were fixed in 1991; sample 2 was fixed in 2000 and samples D7, D9 and F3 were fixed in 2005. The column labeled “RNA” designates the no-reverse transcription control, i.e., the amount of DNA contamination.

FIG. 11 is a graph showing the overall survival of patients receiving Cisplatin/Gem treatment vs. Corrected Relative ERCC1 Expression in NSCLC. Patient Corrected Relative ERCC1 Expression levels lower than the threshold of 6.7×10⁻³ correlated with significantly better survival. While patient Corrected Relative ERCC1 Expression levels higher than the threshold of 6.7×10⁻³ correlated with significantly worse survival. (P=0.009 Log rank test).

FIG. 12 is a chart illustrating how to calculate Corrected Relative ERCC1 expression relative to an internal control gene. The chart contains data obtained with two test samples, (unknowns 1 and 2), and illustrates how to determine the uncorrected gene expression data (UGE). The chart also illustrates how to normalize UGE generated by the TaqMan® instrument with known relative ERCC1 values determined by pre-TaqMan® technology. This is accomplished by multiplying UGE to a correction factor K_{ERCC1}. The internal control gene in the figure is β-actin and the calibrator RNA is Human Liver Total RNA (Stratagene, Cat. #735017).

FIG. 13 is a table showing the demographic details of the 56 patients in the study, tumor stage and cell types. The median number of treatment cycles received was 3 (range 1 6). Fourteen patients (25%) had previously received chemotherapy, mostly (9 patients) taxane therapy alone or in combination with DDP or carboplatin. Three of the 56 patients had received radiotherapy and 5 patients had under gone surgical resection of the primary tumor.

FIG. 14 is a table showing patients with Corrected ERCC1 expression levels below the threshold had a significantly longer median survival of 61.6 weeks (95% C.I. 42.4, 80.7 weeks) compared to 20.4 weeks (95% C.I. 6.9, 33.9 weeks) for patients with Corrected ERCC1 levels above the threshold. Adjusted for tumor stage, the log rank statistic for the association between low or high ERCC1 expression and overall survival was 3.97 and the P value was 0.046. The unadjusted log rank results are shown in this figure. Also shown are factors that were significantly associated with overall survival on univariable analysis using Kaplan Meier survival curves and the log rank test. These were the presence of pretreatment weight loss and the ECOG performance status. Patient age (P=0.18), sex (P=0.87), tumor stage (P=0.99), tumor cell type (P=0.63), and presence of pleural effusion (P=0.71) were not significant prognostic factors for overall survival. Corrected Relative ERCC1 Expression level, ECOG performance status, and weight loss remained significant prognostic factors for survival in the Cox proportional hazards regression model multivariable analysis. P values for a Cox regression model stratified on tumor stage were 0.038 for ERCC1, 0.017 for weight loss, and 0.02 for ECOG performance status (PS 0 versus 1 or 2).

FIG. 15 is a chart illustrating how to calculate DPD expression relative to an internal control gene. The chart contains data obtained with two test samples, (unknowns 1 and 2), and illustrates how to determine the uncorrected gene expression data (UGE) UCG. The chart also illustrates how to normalize UGE generated by the Taqman instrument with previously published DPD values. This is accomplished by multiplying UGE to a correction factor K_{DPD}. The internal control gene in the figure is β-actin and the calibrator RNA is Universal PE RNA; Cat #4307281, lot #3617812014 from Applied Biosystems.

FIG. 16 shows boxplots of relative corrected DPD expression levels for specimens of each histologic type. The boxes show the 25th and 75th percentile (interquartile) ranges. Median values are shown as a horizontal bar within each box. The whiskers show levels outside the 25th and 75th percentiles but exclude far outlying values, which are shown above the boxes.

FIG. 17 is a graph showing the estimated probability of survival and survival in months of colorectal adenocarcinoma tumor carrying patients with high (greater than about 7.5×10⁻³ times β-actin gene expression; n=7) and low (less than about 7.5×10⁻³ times β-actin gene expression; n=43) corrected TS expression levels receiving 5-FU and oxaliplatin therapeutic regimen.

FIG. 18 is a graph showing the estimated probability of survival and survival in months of colorectal adenocarcinoma tumor carrying patients with high (greater than about 4.9×10⁻³ times β-actin gene expression; n=10) and low (less than about 4.9×10⁻³ times P-actin gene expression; n=40) corrected ERCC1 expression levels receiving 5-FU and oxaliplatin therapeutic regimen.

FIG. 19 is a graph showing the estimated probability of survival and survival in months of colorectal adenocarcinoma tumor carrying patients with high (TS expression greater than about 7.5×10⁻³ times β-actin gene expression and ERCC1 greater than about 4.9×10⁻³ times β-actin gene expression; n=14) and low (TS expression less than about 7.5×10⁻³ times β-actin gene expression and ERCC1 less than about 4.9×10⁻³ times β-actin gene expression; n=36) corrected TS and ERCC1 expression levels receiving 5-FU and oxaliplatin therapeutic regimen.

FIG. 20 is a table showing the survival of oxaliplatin/5-FU treated colorectal cancer patients relative to ERCC1 and TS expression analyzed by univariate analysis.

FIG. 21 is a table showing the survival of oxaliplatin/5-FU treated colorectal cancer patients relative to ERCC1 and TS expression analyzed by stratified analysis.

FIG. 22 is a graph showing the response of colorectal adenocarcinoma tumor carrying patients treated with a 5-FU and oxaliplatin chemotherapeutic regimen relative to. Patients were classified into those with progressive disease (PD), partial response (PR), and stable disease (SD). Patients with low levels of both TS and ERCC1 expression had the best response.

FIG. 23 is a chart illustrating how to calculate ERCC1 expression relative to an internal control gene. The chart contains data obtained with two test samples, (unknowns 1 and 2), and illustrates how to determine the uncorrected gene expression data (UGE). The chart also illustrates how to normalize UGE generated by the TaqMan®. instrument with known relative ERCC1 values determined by pre-TaqMan®. technology. This is accomplished by multiplying UGE to a correction factor K_{ERCC1}. The internal control gene in the figure is β-actin and the calibrator RNA is Human Liver Total RNA (Stratagene, Cat. #735017).

FIG. 24 is a chart illustrating how to calculate TS expression relative to an internal control gene. The chart contains data obtained with two test samples, (unknowns 1 and 2), and illustrates how to determine the uncorrected gene expression data (UGE). The chart also illustrates how to normalize UGE generated by the TaqMan® instrument with previously published TS values. This is accomplished by multiplying UGE to a correction factor K_{TS}. The internal control gene in the figure is β-actin and the calibrator RNA is Universal PE RNA; Cat #4307281, lot #3617812014 from Applied Biosystems.

FIG. 25 is a chart illustrating how to calculate EGFR expression relative to an internal control gene. The chart contains data obtained with two test samples, (unknowns 1 and 2), and illustrates how to determine the uncorrected gene expression data (UGE). The chart also illustrates how to normalize UGE generated by the TaqMan® instrument with known relative EGFR values determined by pre-TaqMan® technology. This is accomplished by multiplying UGE to a correction factor K_{EGFR}. The internal control gene in the figure is β-actin and the calibrator RNA is Human Liver Total RNA (Stratagene, Cat #735017).

FIG. 26 is a chart illustrating how to calculate HER2-neu expression relative to an internal control gene. The chart contains data obtained with two test samples, (unknowns 1 and 2), and illustrates how to determine the uncorrected gene expression data (UGE). The chart also illustrates how to normalize UGE generated by the TaqMan® instrument with previously published HER2-neu values. This is accomplished by multiplying UGE to a correction factor K_{HER2-neu}. The internal control gene in the figure is β-actin and the calibrator RNA is Human Liver Total RNA (Stratagene, Cat #735017).

### DETAILED DESCRIPTION

The invention provides a method for isolating long fragment RNA from fixed tissue specimens. Methods of the present invention are suitable for a wide variety of nucleic acid containing biological samples. Methods of the invention are particularly useful in isolating RNA from fixed tumor tissue specimens. Biological samples are often fixed with fixatives such as formalin (formaldehyde) (including Bouin fixative) and glutaraldehyde. Tissue samples fixed using other fixation techniques such as alcohol immersion (Battifora and Kopinski, J. Histochem. Cytochem. (1986) 34:1095) are also suitable. The tissue samples may also be embedded in paraffin. Most commonly, tissue samples are preserved as formalin-fixed paraffin-embedded (FFPE) samples.

Long fragment RNA is defined herein as RNA longer than 100 nt. Preferably, the RNA is about 150 nt in length or longer, more preferably about 200 nt in length or longer and most preferably, about 300 nt in length or longer. In certain embodiments, long fragment RNA is about 400 nt or longer. Long fragment RNA may also include 1000 nt or longer RNA fragments.

Generally, an elevated temperature for some specified time of incubation is necessary to extract macromolecules such as RNA from FFPE specimens. Two general procedures have emerged for achieving recovery of RNA from paraffin: incubation of the FFPE sample either in the presence of a chaotropic agent such as a guanidinium salt or incubation in the presence of proteinase K, an enzyme that degrades proteins and presumably helps to liberate RNA from a protein matrix. Many variations of these basic methods are known. However, no previous study or method has been aimed specifically toward the extraction of longer RNA fragments from FFPE samples. It is a generally held concept that RNA molecules degrade with increasing temperature and increasing time of exposure to an elevated temperature. However, the quantitative relationships between temperature/time and maximum yield of high quality/long fragment RNA material extracted from FFPE was not known or generally appreciated. Furthermore, it was also not known that there may be a threshold temperature at which the RNA does not degrade appreciably due to thermal effects alone.

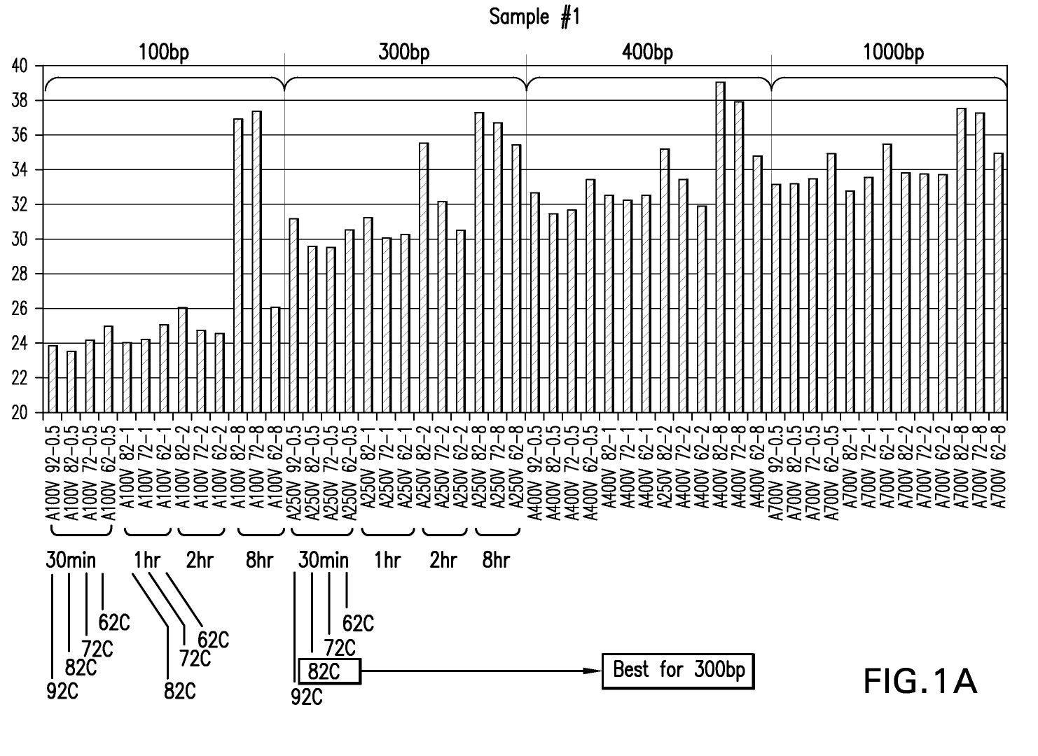

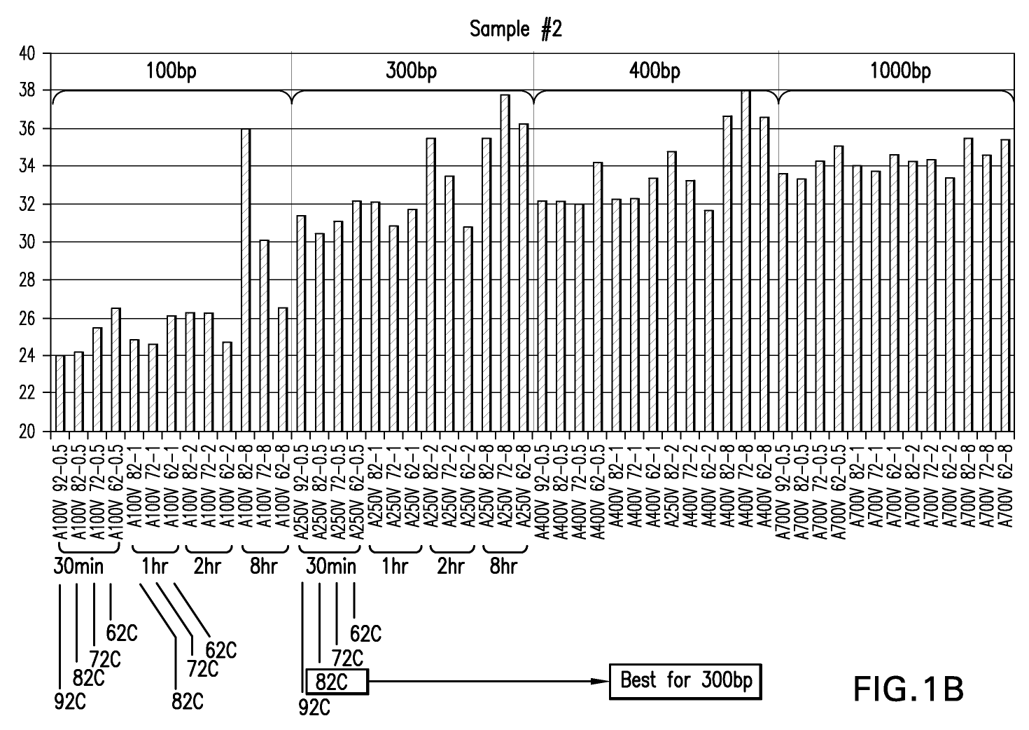

The present inventors have determined that both temperature and time have an effect on RNA quality, but that there is not necessarily a correlation in the effects of temperature and time on short fragment RNA (100 nt or less) to the effects of temperature and time on long fragment RNA (fragments over 100 nt in length, such as 300 nt length fragments). FIGS. 1A and 1B provide the results where FFPE samples were incubated at various temperatures (92, 82, 72 and 62° C.) for various times (0.5, 1, 2, and 8 hours). The Y axis is the Ct value. The Ct value is related to the amount of PCR product and, therefore, relates to the original amount of target present in the PCR reactions. The relation is an inverse one, i.e., a larger Ct number indicates less target RNA originally present. The results shown in FIG. 1 confirm that 300 nt RNA is temperature-sensitive because the yields at 92° C. were lower than at lower temperatures even at the shortest time point of 30 min. However, for 100 nt RNA, at the 30 minute and one hour incubation times, the yield was the lowest at 62° C. and not 92° C. The 300 nt length species are much less abundant than the 100 nt species (about 6 Ct cycles; 2⁶=64-fold) even at short incubation time. In addition, the 300 nt species are more heat sensitive (the Ct cycles increasing by 6 (2⁶=64-fold decrease in yield)) in going from 0.5 hr to 2 hr at 82° C., compared to an increase of 2 cycles (2²=4-fold decrease) for the 100 nt RNA under the same conditions. The changes in yield are minimal at 62° C. over all incubation time periods, indicating the presence of a threshold temperature below which RNA is relatively stable and suggesting that incubation at a lower temperature for longer times would be more productive for optimal isolation of higher molecular weight RNA. Temperatures higher than 62° C. cause considerably more degradation of RNA over time and while the total yield of RNA may increase somewhat at higher temperatures, the yield of long fragment RNA decreases.

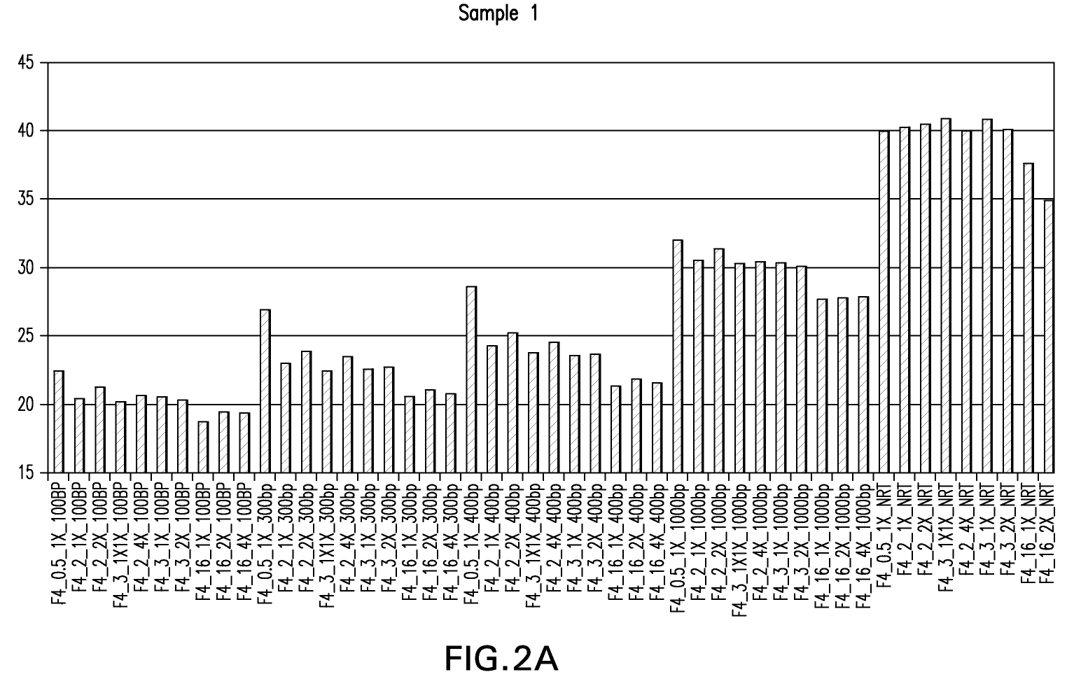

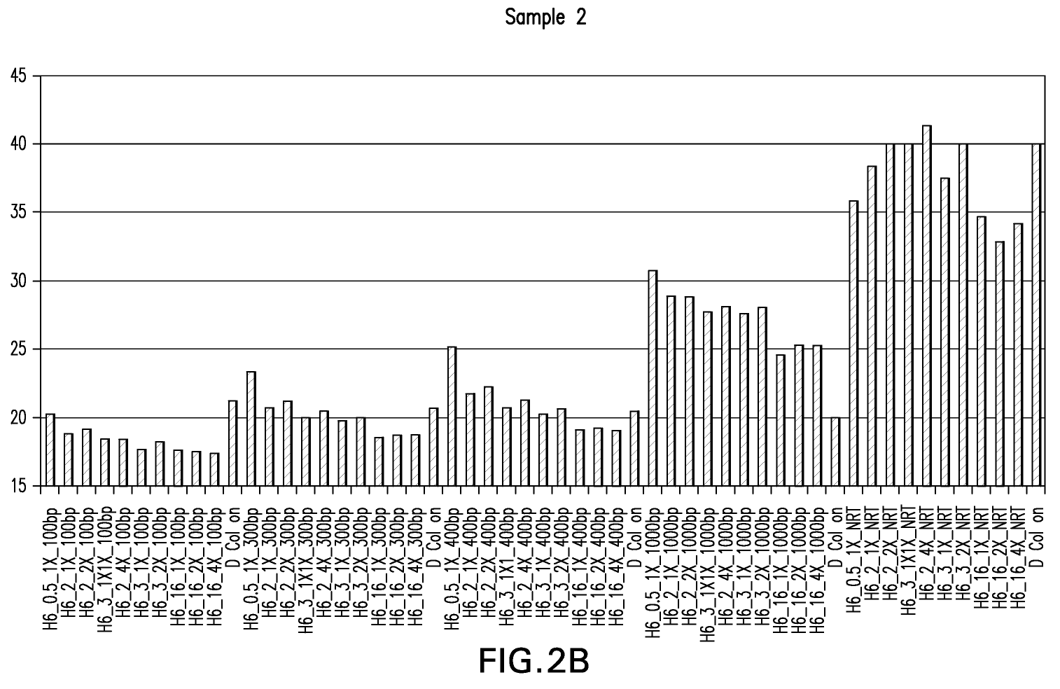

FIGS. 2A and 2B illustrate the effect of heating time on the amount of recovered RNA (yield) of each length of RNA species at 50° C. These figures show that the yield of all sizes of RNA increased when incubated at a longer time. As illustrated in FIG. 2, FFPE samples of tumor tissue were heated in the presence of proteinase K at 50° C. for time periods that varied from 0.5 hr to 16 hr. For example, the designation of F4_{—}0.5_{—}1× 100 BP, means that sample F4 was heated for 0.5 hours and is a 100 nt length fragment. The yield of all sizes of RNA fragments increased with the increase of incubation time. The yield of 100 nt fragments increased by over 10-fold (ca. 3.5 PCR cycles) while that of 300 nt fragments increased by almost 2⁶ (corresponding to a decrease of 6 PCR cycles) or about 60-fold in going from the short to the long incubation times. This was similarly seen for the 400 nt fragments. Somewhat less of an increase in yield was seen for the 1000 nt fragments (about 10-fold), which may indicate increased extraction perhaps balanced by some degradation of this size at the long incubation times. These data illustrate the considerable time dependence of the RNA extraction from the paraffin matrix and also point out the stability of all RNA fragment lengths at 50° C. under these conditions (as opposed to that at 82° C. as shown in FIG. 1). It is apparent that lower incubation temperatures substantially increased the yield of the 300 nt and greater RNA species not only on an absolute level but on a relative level (i.e., only a 2-fold Ct difference between the 100 bp and the 300 bp species at 16 hr incubation times).

Thus, taking FIGS. 1 and 2 together, the data show that RNA is thermally unstable at 92° C. and that an increased temperature is especially detrimental on long fragment RNA. The figures also show that at elevated temperatures, longer incubation times degrade RNA more substantially than at lower temperatures. However, as seen in FIG. 2, when temperatures are lowered to a more moderate temperature, there is not a time dependent degradation that one would expect to still see.

Accordingly, the present invention provides a method of long fragment RNA isolation from a fixed tissue samples, such as FFPE tissue, wherein the tissue sample is heated at a temperature range of about 45 to about 62° C. for a time period of 3 hours or greater. In other embodiments, the fixed tissue sample is heated at a temperature range of about 44 to about 60° C., in more preferred embodiments from about 48 to about 58° C., in other preferred embodiments from about 48 to about 55° C., in other preferred embodiments from 48 to 52° C., and in most preferred embodiments about 50° C. to about 56° C. One skilled in the art would appreciate that the term “about” is used to encompass small insubstantial temperature variations that may occur in heating baths, heating blocks, and PCR machines and that such small variations from device to device or from lab to lab may have no detrimental or net positive effect, and are encompassed within the scope of the claims. One skilled in the art would appreciate that the term about is meant to indicate that temperature variations near the stated temperature range are encompassed by the scope of the claims, as long as the methods work for their intended use (i.e. isolating long fragment RNA).

The tissue sample is heated at the above discussed temperatures for a time period of anywhere of 2 hours or greater up to 20 hours (and any time in between). For example, the time period may fall within any time between 2 hours and up to 20 hours, and in any range within. For example, the present invention provides heating at a time period from about 4 hours to about 19 hours, about 5 hours to about 17 hours, about 6 hours to about 17 hours, about 7 hours to about 16.5 hours, about 8 hours to about 16.5 hours, about 9 hours to about 16.5, about 10 hours to about 16.25 hours, etc. In preferred embodiments, the time period is about 12 hours to 17 hours, and in more preferred embodiments, the time period is for about 14 to about 16 hours. In most preferred embodiments, the time period is about 16 hours. One skilled in the art would appreciate that the term “about” is meant to indicate that time variations near the stated hour amount are encompassed by the scope of the claims, as long as the methods work for their intended use (i.e. isolating long fragment RNA).

An especially preferred embodiment on the present invention comprises heating a fixed tissue sample (such as FFPE) at about 50° C. for about 16 hours, which maximizes the yield of long fragment RNA, and specifically 300 nt or longer RNA. As discussed above and shown in FIGS. 1 and 2, the RNA is stable for long periods of time at this temperature and, since the extraction of macromolecules from paraffin is also time-dependent, an incubation time of about 16 hr is desirable (and possible at this temperature) to achieve the maximum yield of long fragment RNA.

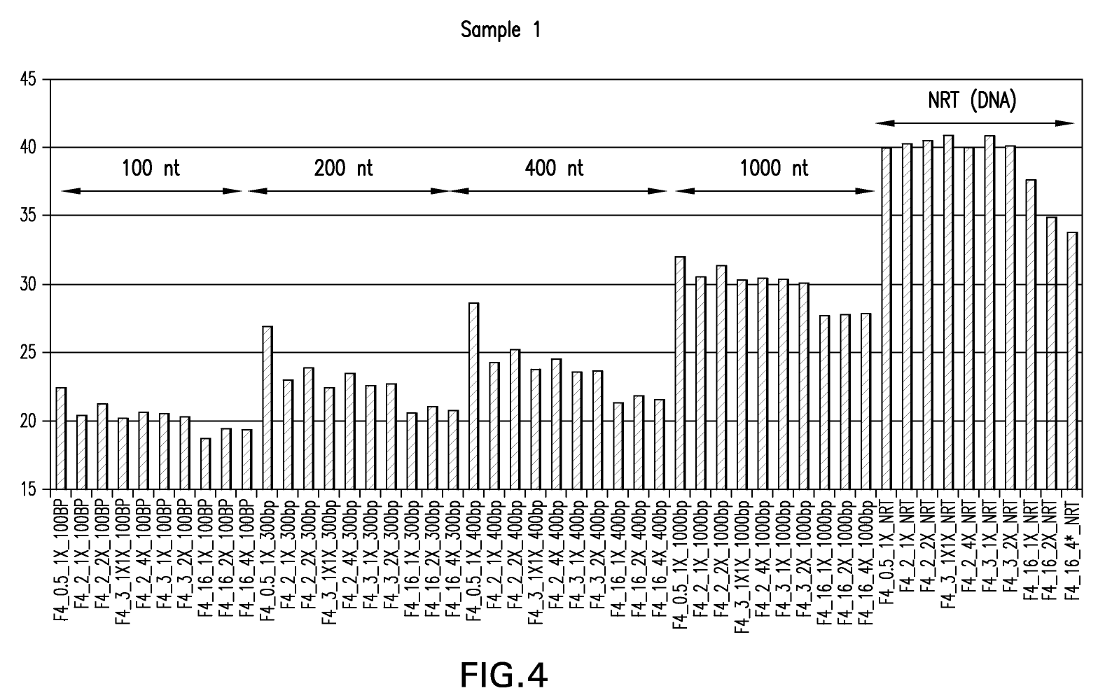

The present method also comprises heating a fixed tissue sample in the presence of proteinase K. As discussed above, proteinase K is useful to extract the maximal amount of RNA. However, if an excess of proteinase K is used in the isolation, it will also liberate more DNA from the matrix, resulting in a higher DNA contamination of the RNA preparation. FIG. 4 shows the effects of varying the amount of proteinase K in the incubation step to remove RNA from the paraffin matrix. For example, the sample labeled F4_{—}16_{—}1×_{—}100 bp, which stands for sample F4, heated for 16 hours, with a 1× (5 μg) amount of proteinase K has a lower CT value than the F4_{—}16_{—}2×_{—}100 bp (2×=10 μg) and the F4_{—}16_{—}4×_{—}100 bp (4×=20 μg) samples. This is also observed in the 300 bp and 400 bp sized RNA (See samples F4_{—}16_{—}1×_{—}300 bp and F4_{—}16_{—}1×_{—}400 bp for having the lowest CT threshold for that sized RNA). Accordingly, a concentration of 5 μg proteinase K/400 μL, (12.5 μg proteinase K/mL) is preferred. However, as shown in Example 19, other amounts of proteinase K can be used successfully (i.e. 0.5×, 1×, 2×, 4×). Alternatives to proteinase K may also be used, including but not limited to, Qiagen® protease, brofasin (a plant proteinase extracted from Bromelia fastiasa), and a cysteine proteinase isolated from carica candamarcensis.

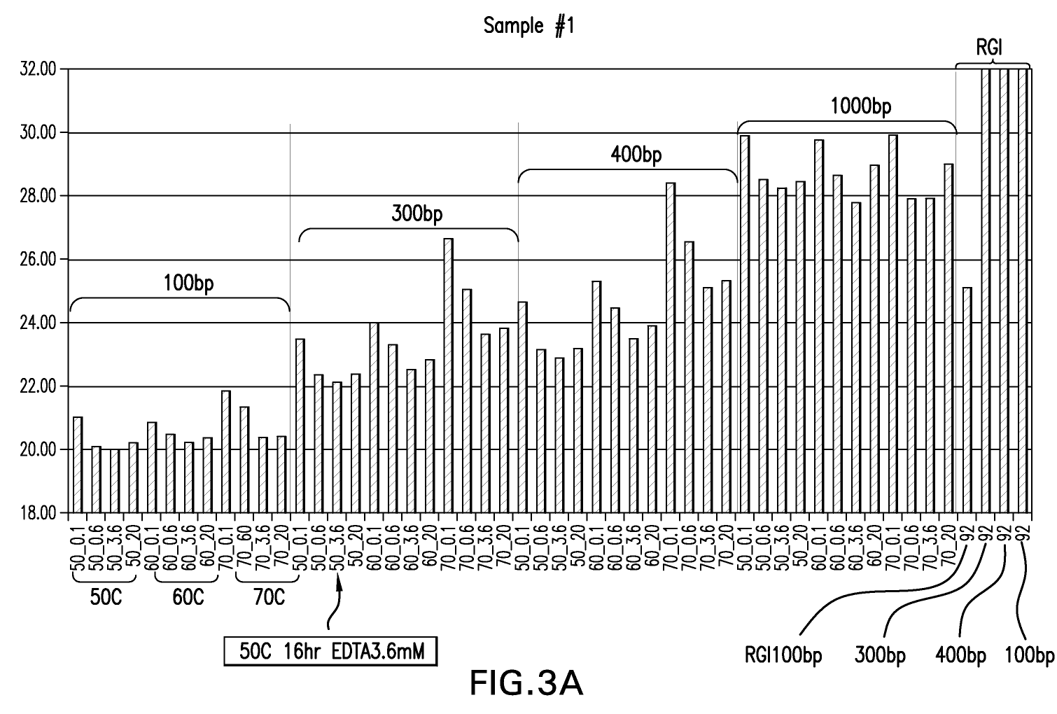

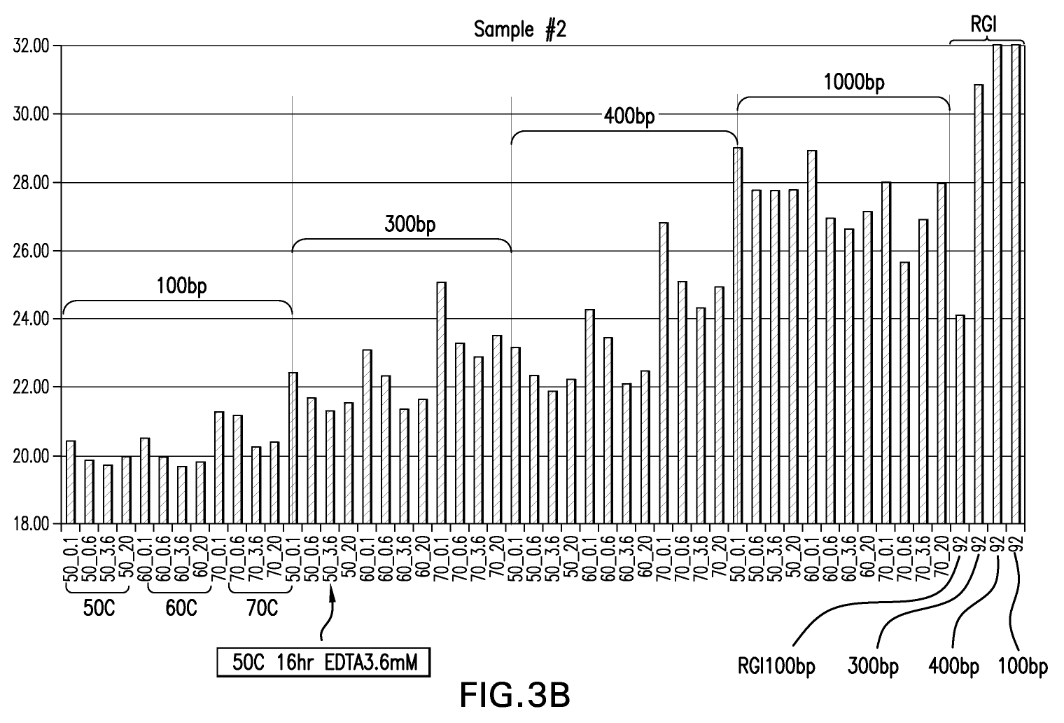

EDTA (ethylene diamine tetraacetic acid), which is used is to chelate divalent or greater metal ions, is often added as a component of extraction buffers. However, a study of known methods that use EDTA in extraction buffers reveal a wide range of concentrations of EDTA in FFPE extraction procedures, which suggests that others did not appreciate that a specific concentration of EDTA existed or was necessary for preventing the effects of divalent metal ions on the RNA molecule. FIG. 3 shows the effects of incubation time and EDTA concentration. FFPE tissue samples were treated according to the methods described in example 1, with the exception that the samples were exposed to different temperatures (50, 60, and 70° C.) during the 16 hr incubation in the presence of proteinase K. Additionally, four different concentrations of EDTA in the extraction solution (0.1, 0.5, 3.6 and 20 mM) was used. FIG. 3A shows that a concentration of 3.6 mM EDTA gave higher yields of long fragment RNA, often increasing the yield by greater than two-fold. FIG. 3 also illustrates that the highest yield of RNA of all sizes was obtained at the 50° C. incubation temperatures (about 25% more than at 60° C. and about 4-fold more than at 70° C.). A low concentration of EDTA (0.1 mM) decreased the yield of RNA by as much as 2-3 fold, while using a high level of 20 mM decreased the yield compared to that at of 3.6 mM about 25%.

Accordingly, the present method further provides the use of a chelator, such as EDTA, in the extraction solution. Chelating agents are well known organic compounds that are capable of forming complexes of multivalent metal ions. As such, other chelators besides EDTA may be employed in the methods of the present invention. The chelator may be chosen from those commonly in use. For example, EDTAs, EGTAs, citrates (such as sodium citrate), citric acids, salicylic acids, salts of salicylic acids, phthalic acids, 2,4-pentanedines, histidines, histidinol dihydrochlorides, 8-hydroxyquinolines, 8-hydroxyquinoline, citrates and o-hydroxyquinones are representative of chelators known in the art. In a preferred embodiment EDTA or sodium citrate is employed. See example 15.

The chelator may be present at a concentration of about 0.1 mM to about 15 mM, and any concentration or range within. Preferred embodiments comprise a chelator at a concentration of about 2.5 mM to about 5.0 mM. A preferred embodiment comprises a chelator at a concentration of about 3.0 mM to about 4.0 mM. More preferred embodiments comprise a chelator at a concentration of about 3.25 to about 3.75 mM. Most preferably, the chelator is present at a concentration of about 3.6 mM and in most preferred embodiments, the chelator is EDTA or sodium citrate and is present at about from about 0.6 mM to about 3.6 mM, or is present about 3.6 mM. One skilled in the art would appreciate that the term “about” is meant to indicate that variations near the stated mM amount are encompassed by the scope of the claims, as long as the methods work for their intended use (i.e. isolating long fragment RNA).

A disadvantage of long incubation times such as used in the present invention (as opposed to short incubation times at higher temperatures with chaotropic agents) is that an appreciable amount of DNA can be co-extracted with the RNA, leading to potential problems with the RNA analysis described in the background section. DNA contamination is often dealt with by treating the sample with deoxyribonuclease (DNAse) at a post-extraction stage of the procedure. While this reagent can be effective at reducing the amount of DNA, it also may reduce the amount of RNA isolated by four- to eight-fold, which can be a serious loss for analysis of a specimen of an already non-abundant tissue content. Accordingly, the present invention also provides a method of isolation that does not require the use of a DNAase and in its place utilizes a double-phenol/chloroform extraction method following the proteinase K step. The double phenol/chloroform extraction involves using a chaotropic agent that specifically removes the bulk of the DNA while preserving the yield of RNA.

In preferred embodiments, double phenol/chloroform extraction with a chaotropic agent co-isolates less than 10% DNA.

The double phenol/chloroform extraction step comprises performing at least a first and a second phenol/chloroform extraction wherein the second phenol/chloroform extraction comprises a chaotropic agent. Any chaotropic agent may be used. Chaotropic agents stabilize nucleic acids by inhibiting nuclease activity. For example, known chaotropic agents include, but are not limited to urea, guanidinium isothiocyanate, sodium thiocyanate (NaSCN), Guanidine HCl, guanidinium chloride, guanidinium thiocyanate, lithium tetrachloroacetate, sodium perchlorate, rubidium tetrachloroacetate, potassium iodide and cesium trifluoroacetate, among others.

Other methods of removing DNA contamination may be employed (as long as they do not appreciably destroy the long fragment RNA), including but not limited to the use of commercially available products such as purification columns from Qiagen (with our without the use of DNase) and Ambion Turbo DNase free process.

After the removal of the DNA contamination, the RNA is isolated using known procedures, such as ethanol or isopropanol precipitation.

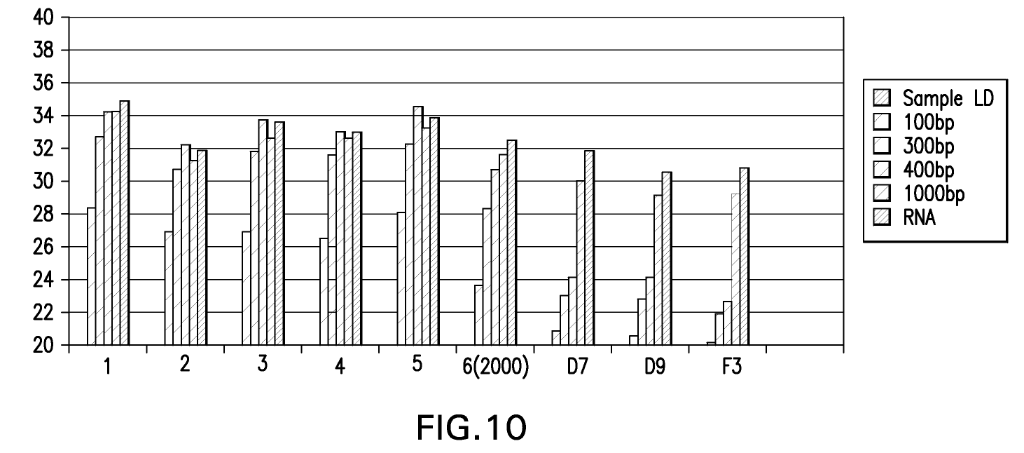

The prevailing belief seems to be that RNA molecules, once formalin-fixed and embedded into the paraffin matrix are stable for indefinite periods of time, so that the age of archival samples is no longer an issue. While it is true that total RNA (all sizes and fragments) decreases only very slowly over time, the present inventors have determined that this is not the case for longer fragments of RNA. The abundance of RNA fragments of 300 nt and higher decreases dramatically in older FFPE specimens. FIG. 10 shows that samples fixed in 1991 (samples 1, 2, 3, 4 and 5) give a lower yield and a lower quality RNA than samples fixed in 2005 (D7, D9 and F3). Thus, for applications in which long fragment RNA is important, the FFPE tissue sample should be 5 years old or less. However, long fragment RNA has been obtained when the sample is 16 years old (data not shown). Accordingly in one embodiment of the present invention, the age of the fixed sample is less than 5 years old. In a more preferred embodiment, the sample is less than 3 years old and in a most preferred embodiment, the sample is less than 2 years old.

RNA isolated by the methods of the invention is suitable for a variety of purposes and molecular biology procedures including, but not limited to: reverse transcription to cDNA; producing radioactively, fluorescently or otherwise labeled cDNA for analysis on gene chips, oligonucleotide microarrays and the like; electrophoresis by acrylamide or agarose gel electrophoresis; purification by chromatography (e.g. ion exchange, silica gel, reversed phase, or size exclusion chromatography); hybridization with nucleic acid probes; and fragmentation by mechanical, sonic or other means.

Often in the field of cancer, expression of biomarkers is a key for diagnosis, as well as determining a therapeutic regimen. The present method can be used to isolate RNA for any desired biomarker. Examples include, but are not limited to, Kras, MMR1, ERCC1, DPD, Gst-pi, EGFR, TS, and Her2-neu.

Accordingly, the present invention provides not only methods of isolating long fragment RNA but also provides RNA isolated by the disclosed methods herein. Another embodiment of the present invention provides cDNA made by copying isolated RNA of the present invention. Those skilled in the art would appreciate that cDNA can readily be made from isolated and purified RNA. Further, another aspect of the present invention provides use of isolated RNA or cDNA made from the isolated RNA to manufacture a microarray or gene chip. The present invention also provides the use of isolated RNA of the present invention in analysis of gene expression or gene copy number for therapeutic or diagnostic purposes, as often occurs in cancer detection/diagnosis and in the field of determining a proper chemotherapeutic regimen based on gene expression levels or gene copy number. Using appropriate PCR primers, the expression level of any messenger RNA can be determined by the methods of the invention. The quantitative RT-PCR technique allows for the comparison of protein expression levels in paraffin-embedded (via immunohistochemistry) with gene expression levels (using RT-PCR) in the same sample.

### ERCCI

Certain embodiments of the present invention reside in part in the finding that the amount of ERCC1 mRNA in a tumor correlates with survival in patients treated with DNA platinating agents. Patients with tumors expressing high levels of ERCC1 mRNA are considered likely to be resistant to platinum-based chemotherapy and thus have lower levels of survivability. Conversely, those patients whose tumors express low amounts of ERCC1 mRNA are likely to be sensitive to platinum-based chemotherapy and have greater levels of survivability. A patient's relative expression of tumor ERCC1 mRNA is judged by comparing it to a predetermined threshold expression level.

Accordingly, one embodiment of the present invention provides a method of quantifying the amount of ERCC1 long fragment mRNA expression in fixed and paraffin-embedded (FPE) tissue relative to gene expression of an internal control. The present inventors have developed oligonucleotide primers that allow accurate assessment of ERCC1 expression in tissues that have been fixed and embedded. The invention provides the use of oligonucleotide primers, ERCC1-504F (SEQ ID NO: 1), ERCC1-574R (SEQ ID NO: 2), or oligonucleotide primers substantially identical thereto, preferably are used together with long fragment RNA extracted from fixed and paraffin embedded (FPE) tumor samples (preferably using the extraction methods of the present invention). This measurement of ERCC1 gene expression may then be used for prognosis of platinum-based chemotherapy. See for example U.S. Pat. No. 6,573,052, incorporated by reference.

As such, one embodiment of the invention involves first, extraction of long fragment RNA from an FPE sample and second, determination of the content of ERCC1 mRNA in the sample by using a pair of oligonucleotide primers, preferably oligionucleotide primer pair ERCC1-504F (SEQ ID NO: 1) and ERCC1-574R (SEQ ID NO: 2), or oligonucleotides substantially identical thereto, for carrying out reverse transcriptase polymerase chain reaction. Preferably RNA is extracted from the FPE cells by any of the methods disclosed herein.

The present methods can be applied to any type of tissue from a patient. For examination of resistance of tumor tissue, it is preferable to examine the tumor tissue. In a preferred embodiment, a portion of normal tissue from the patient from which the tumor is obtained, is also examined. Patients whose normal tissues are expected to be resistant to platinum-based chemotherapeutic compounds, i.e., show a high level of ERCC1 gene expression, but whose tumors are expected to be sensitive to such compounds, i.e., show a low level of ERCC1 gene expression, may then be treated with higher amounts of the chemotherapeutic composition.

Patients showing a level of ERCC1 gene expression below the threshold level, may be treated with higher amounts of the chemotherapeutic composition because they are expected to have greater survivability than patients with tumors expressing a level of ERCC1 gene expression above the threshold level. Alternatively, the clinician may determine that patients with tumors expressing a level of ERCC1 gene expression above the threshold level may not derive any significant benefit from chemotherapy given their low expected survivability.

The methods of the present invention can be applied over a wide range of tumor types. This allows for the preparation of individual “tumor expression profiles” whereby expression levels of ERCC1 are determined in individual patient samples and response to various chemotherapeutics is predicted. Preferably, the methods of the invention regarding ERCC1 are applied to solid tumors, most preferably Non-Small Cell Lung Cancer (NSCLC) tumors. For application of some embodiments of the invention to particular tumor types, it is preferable to confirm the relationship of ERCC1 gene expression levels to survivability by compiling a dataset that enables correlation of a particular ERCC1 expression and clinical resistance to platinum-based chemotherapy.

A “predetermined threshold level,” as defined herein, as it relates to ERCC1 is a corrected relative level of ERCC1 tumor expression above which it has been found that tumors are likely to be resistant to a platinum-based chemotherapeutic regimen. Tumor expression levels below this threshold level are likely to be found in tumors sensitive to platinum-based chemotherapeutic regimen. The range of corrected relative expression of ERCC1, expressed as a ratio of ERCC1:β-actin, among tumors responding to a platinum-based chemotherapeutic regimen is less than about 6.7×10⁻³. Tumors that do not respond to a platinum-based chemotherapeutic regimen have relative expression of ERCC1:β-actin ratio above about 6.7×10⁻³. See Example 7.

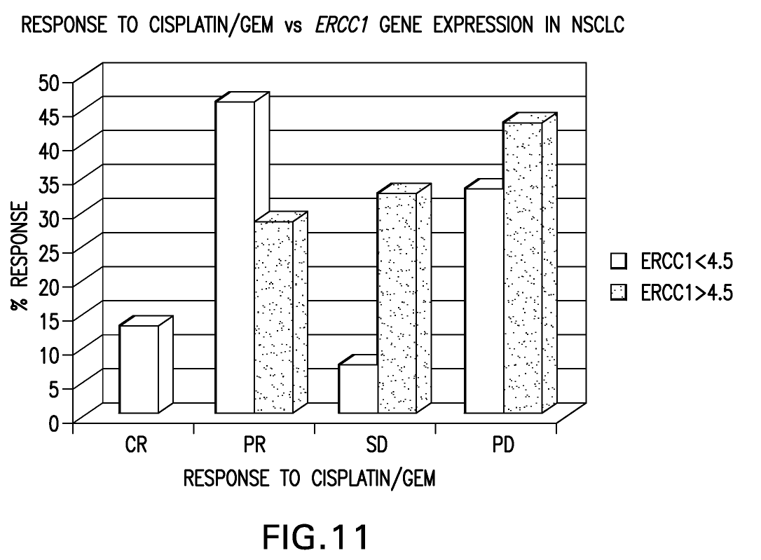

A “predetermined threshold level” is further defined as it relates to ERCC1 as tumor corrected relative ERCC1 expression levels above which patients receiving a platinum-based chemotherapeutic regimen are likely to have low survivability. Tumor corrected relative ERCC1 expression levels below this threshold level in patients receiving a platinum-based chemotherapeutic regimen correlate to high patient survivability. The threshold corrected relative ERCC1 expression, expressed as a ratio of ERCC1:β-actin, is about 6.7×10⁻³. See FIG. 11, Example 7. However, the present invention is not limited to the use of β-actin as an internal control gene.

RNA is extracted from the FPE tissues by any of the methods of the present invention as discussed herein. Fixed and paraffin-embedded (FPE) tissue samples as described herein refers to storable or archival tissue samples. RNA may be isolated from an archival pathological sample or biopsy sample which is first deparaffinized. An exemplary deparaffinization method involves washing the paraffinized sample with an organic solvent, such as xylene, for example. Deparaffinized samples can be rehydrated with an aqueous solution of a lower alcohol. Suitable lower alcohols, for example include, methanol, ethanol, propanols, and butanols. Deparaffinized samples may be rehydrated with successive washes with lower alcoholic solutions of decreasing concentration, for example. Alternatively, the sample is simultaneously deparaffinized and rehydrated. RNA is then extracted from the sample.

The quantification of ERCC1 mRNA from purified total mRNA from fresh, frozen or fixed is preferably carried out using reverse-transcriptase polymerase chain reaction (RT-PCR) methods common in the art, for example. Other methods of quantifying of ERCC1 mRNA include for example, the use of molecular beacons and other labeled probes useful in multiplex PCR. Additionally, the present invention envisages the quantification of ERCC1 mRNA via use of PCR-free systems employing, for example fluorescent labeled probes similar to those of the Invader® Assay (Third Wave Technologies, Inc.). Most preferably, quantification of ERCC1 cDNA and an internal control or house keeping gene (e.g. β-actin) is done sing a fluorescence based real-time detection method (ABI PRISM 7700 or 7900 Sequence Detection System [TaqMan®], Applied Biosystems, Foster City, Calif.) or similar system as described by Heid et al., (Genome Res 1996; 6:986 994) and Gibson et al. (Genome Res 1996; 6:995 1001). The output of the ABI 7700 (TaqMan® Instrument) is expressed in Ct's or “cycle thresholds.” With the TaqMan® system, a highly expressed gene having a higher number of target molecules in a sample generates a signal with fewer PCR cycles (lower Ct) than a gene of lower relative expression with fewer target molecules (higher Ct).

As used herein, a “house keeping” gene or “internal control” is meant to include any constitutively or globally expressed gene whose presence enables an assessment of a target mRNA levels (such as, but not limited to ERCC1, TS, DPD, Her2-neu, Gst-pi, RRM1, Kras, etc.). Such an assessment comprises a determination of the overall constitutive level of gene transcription and a control for variations in RNA recovery. “House-keeping” genes or “internal controls” can include, but are not limited to the cyclophilin gene, β-actin gene, the transferrin receptor gene, GAPDH gene, and the like. Most preferably, the internal control gene is β-actin gene as described by Eads et al., Cancer Research 1999; 59:2302 2306.

A control for variations in RNA recovery requires the use of “calibrator RNA.” The “calibrator RNA” is intended to be any available source of accurately pre-quantified control RNA. Preferably, Human Liver Total RNA (Stratagene, Cat. #735017) is used.

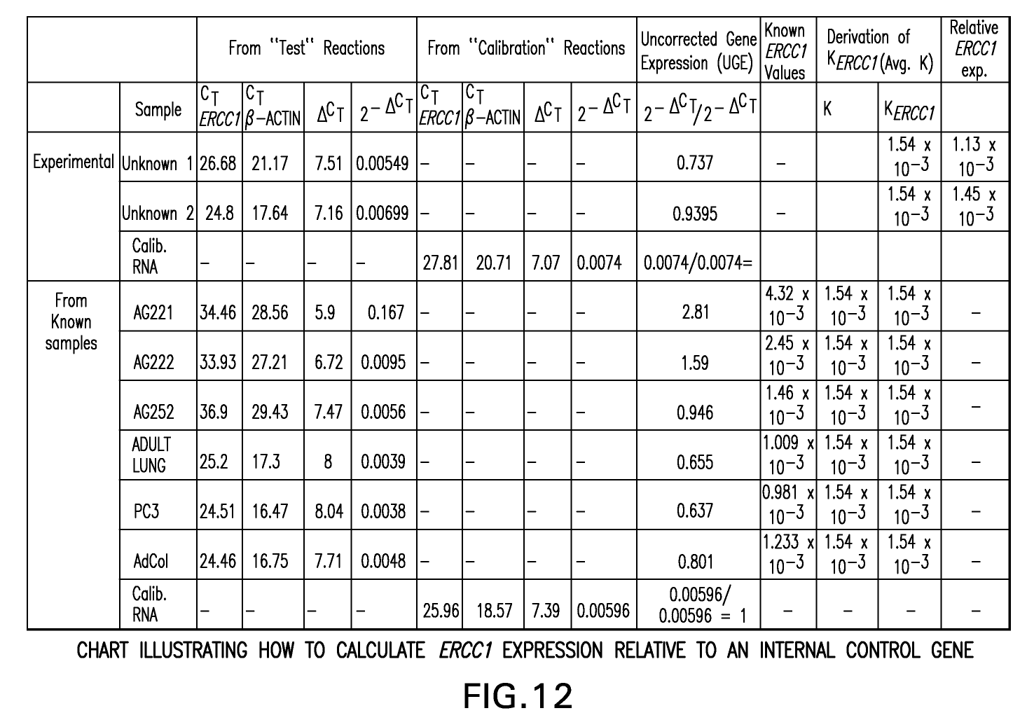

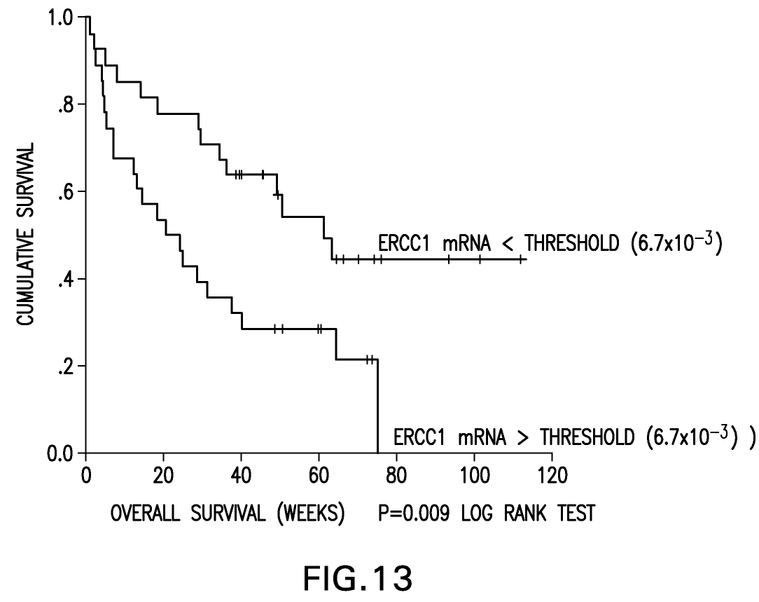

“Uncorrected Gene Expression (UGE)” as used herein refers to the numeric output of a target gene expression relative to an internal control gene generated by the TaqMan® instrument. The equation used to determine UGE for ERCC1 is shown in Example 6, and illustrated with sample calculations in FIG. 12.

A further aspect of this invention provides a method to normalize uncorrected gene expression (UGE) values acquired from the TaqMan® instrument with “known relative gene expression” values derived from non-TaqMan® technology. Preferably, the known non-TaqMan® derived relative ERCC1:β-actin expression values are normalized with TaqMan® derived ERCC1 UGE values from a tissue sample.

“Corrected Relative ERCC1 Expression” as used herein refers to normalized ERCC1 expression whereby UGE is multiplied with a ERCC1 specific correction factor (K_{ERCC1}), resulting in a value that can be compared to a known range of ERCC1 expression levels relative to an internal control gene. Example 6 and FIG. 12 illustrate these calculations in detail. These numerical values allow the determination of whether or not the “Corrected Relative ERCC1 Expression” of a particular sample falls above or below the “predetermined threshold” level. The predetermined threshold level of Corrected Relative ERCC1 Expression to β-actin level is about 6.7×10⁻³. K_{ERCC1} specific for ERCC1, the internal control β-actin and calibrator Human Liver Total RNA (Stratagene, Cat. #735017), is 1.54×10⁻³.

“Known relative gene expression” values are derived from previously analyzed tissue samples and are based on the ratio of the RT-PCR signal of a target gene to a constitutively expressed internal control gene (e.g. β-Actin, GAPDH, etc.). Preferably such tissue samples are formalin fixed and paraffin-embedded (FPE) samples and RNA is extracted from them according to methods described herein. To quantify gene expression relative to an internal control standard quantitative RT-PCR technology known in the art is used. Pre-TaqMan® technology PCR reactions are run for a fixed number of cycles (i.e., 30) and endpoint values are reported for each sample. These values are then reported as a ratio of ERCC1 expression to β-actin expression. See U.S. Pat. No. 5,705,336 to Reed et al.

K_{ERCC1} may be determined for an internal control gene other than β-actin and/or a calibrator RNA different than Human Liver Total RNA (Stratagene, Cat. #735017). To do so, one must calibrate both the internal control gene and the calibrator RNA to tissue samples for which ERCC1 expression levels relative to that particular internal control gene have already been determined (i.e., “known relative gene expression”). Preferably such tissue samples are formalin fixed and paraffin-embedded (FPE) samples and RNA is extracted using a method disclosed herein. Such a determination can be made using standard pre-TaqMan®, quantitative RT-PCR techniques well known in the art. Upon such a determination, such samples have “known relative gene expression” levels of ERCC1 useful in the determining a new K_{ERCC1} specific for the new internal control and/or calibrator RNA as described in Example 6.

Generally, any oligonucleotide pair that flanks a region of ERCC1 gene may be used to carry out the methods of the invention. Primers hybridizing under stringent conditions to a region of the ERCC1 gene for use in the present invention will amplify a product between 20 1000 base pairs, preferably 100-400 base pairs, most preferably about 200-400 base pairs.

The invention provides specific oligonucleotide primers pairs and oligonucleotide primers substantially identical thereto, that allow particularly accurate assessment of ERCC1 expression in FPE tissues. Preferred oligonucleotide primers include, ERCC1-504F (SEQ ID NO: 1) and ERCC1-574R(SEQ ID NO: 2), (also referred to herein as the oligonucleotide primer pair ERCC1) and oligonucleotide primers substantially identical thereto. The oligonucleotide primers ERCC1-504F (SEQ ID NO: 1) and ERCC1-574R, (SEQ ID NO: 2) hybridize to the ERCC1 gene under stringent conditions and have been shown to be particularly effective for measuring ERCC1 mRNA levels using RNA extracted from the FPE cells by any of the methods for mRNA isolation, especially by the methods disclosed herein.

“Substantially identical” in the nucleic acid context as used herein, means hybridization to a target under stringent conditions, and also that the nucleic acid segments, or their complementary strands, when compared, are the same when properly aligned, with the appropriate nucleotide insertions and deletions, in at least about 60% of the nucleotides, typically, at least about 70%, more typically, at least about 80%, usually, at least about 90%, and more usually, at least, about 95 to 98% of the nucleotides. Selective hybridization exists when the hybridization is more selective than total lack of specificity. See, Kanehisa, Nucleic Acids Res., 12:203 213 (1984).

This invention includes substantially identical oligonucleotides that hybridize under stringent conditions (as defined herein) to all or a portion of the oligonucleotide primer sequence of ERCC1-504F (SEQ ID NO: 1), its complement or ERCC1-574R (SEQ ID NO: 2), or its complement.

Under stringent hybridization conditions, only highly complementary, i.e., substantially similar nucleic acid sequences hybridize. Preferably, such conditions prevent hybridization of nucleic acids having 4 or more mismatches out of 20 contiguous nucleotides, more preferably 2 or more mismatches out of 20 contiguous nucleotides, most preferably one or more mismatch out of 20 contiguous nucleotides.

The hybridizing portion of the nucleic acids is typically at least 10 (e.g., 15) nucleotides in length. The hybridizing portion of the hybridizing nucleic acid is at least about 80%, preferably at least about 95%, or most preferably about at least 98%, identical to the sequence of a portion or all of the oligonucleotide primers provided herein or their complement.

Hybridization of the oligonucleotide primer to a nucleic acid sample under stringent conditions is defined below. Nucleic acid duplex or hybrid stability is expressed as a melting temperature (Tₘ), which is the temperature at which the probe dissociates from the target DNA. This melting temperature is used to define the required stringency conditions. If sequences are to be identified that are substantially identical to the probe, rather than identical, then it is useful to first establish the lowest temperature at which only homologous hybridization occurs with a particular concentration of salt (e.g. SSC or SSPE). Then assuming that 1% mismatching results in a 1° C. decrease in Tₘ, the temperature of the final wash in the hybridization reaction is reduced accordingly (for example, if sequences having >95% identity with the probe are sought, the final wash temperature is decrease by 5° C.). In practice, the change in Tₘ can be between 0.5° C. and 1.5° C. per 1% mismatch.

Stringent conditions involve hybridizing at 68° C. in 5×SSC/5× Denhart's solution/1.0% SDS, and washing in 0.2×SSC/0.1% SDS at room temperature. Moderately stringent conditions include washing in 3×SSC at 42° C. The parameters of salt concentration and temperature be varied to achieve optimal level of identity between the primer and the target nucleic acid. Additional guidance regarding such conditions is readily available in the art, for example, Sambrook, Fischer and Maniatis, Molecular Cloning, a laboratory manual, (2nd ed.), Cold Spring Harbor Laboratory Press, New York, (1989) and F. M. Ausubel et al eds., Current Protocols in Molecular Biology, John Wiley and Sons (1994).

Oligonucleotide primers disclosed herein are capable of allowing accurate assessment of ERCC1 gene expression in a fixed or fixed and paraffin embedded tissue, as well as frozen or fresh tissue. This is despite the fact that RNA derived from FPE samples is more fragmented relative to that of fresh or frozen tissue. Thus, the methods of the invention are suitable for use in assaying ERCC1 expression levels in FPE tissue where previously there existed no way to assay ERCC1 gene expression using fixed tissues.

From the measurement of the amount of ERCC1 mRNA that is expressed in the tumor, the skilled practitioner can make a prognosis concerning clinical resistance of a tumor to a particular genotoxin or the survivability of a patient receiving a particular genotoxin. An exemplary platinum-based chemotherapy or a chemotherapy inducing a similar type of DNA damage, is genotoxin.

Platinum-based chemotherapies cause a “bulky adduct” of the DNA, wherein the primary effect is to distort the three-dimensional conformation of the double helix. Such compounds are meant to be administered alone, or together with other chemotherapies such as gemcitabine (Gem) or 5-Fluorouracil (5-FU).

Platinum-based genotoxic chemotherapies comprises heavy metal coordination compounds which form covalent DNA adducts. Generally, these heavy metal compounds bind covalently to DNA to form, in pertinent part, cis-1,2-intrastrand dinucleotide adducts. Generally, this class is represented by cis-diamminedichloroplatinum (II) (cisplatin), and includes cis-diammine-(1,1-cyclobutanedicarboxylato) platinum(II) (carboplatin), cis-diammino-(1,2-cyclohexyl) dichloroplatinum(II), and cis-(1,2-ethylenediammine) dichloroplatinum(II). Platinum first agents include analogs or derivatives of any of the foregoing representative compounds.

Tumors currently manageable by platinum coordination compounds include testicular, endometrial, cervical, gastric, squamous cell, adrenocortical and small cell lung carcinomas along with medulloblastomas and neuroblastomas. Trans-Diamminedichloroplatinum (II) (trans-DDP) is clinically useless owing, it is thought, to the rapid repair of its DNA adducts. The use of trans-DDP as a chemotherapeutic agent herein likely would provide a compound with low toxicity in nonselected cells, and high relative toxicity in selected cells. In a preferred embodiment, the platinum compound is cisplatin.

Many compounds are commonly given along with platinum-based chemotherapy agents. For example, BEP (bleomycin, etoposide, cisplatin) is used for testicular cancer, MVAC (methotrexate, vinblastine, doxorubicin, cisplatin) is used for bladder cancer, MVP (mitomycin C, vinblastine, cisplatin) is used for non-small cell lung cancer treatment. Many studies have documented interactions between platinum-containing agents. Therapeutic drug synergism, for example, has been reported for many drugs potentially included in a platinum based chemotherapy. A very short list of recent references for this include the following: Okamoto et al., Urology 2001; 57:188-192.; Tanaka et al., Anticancer Research 2001; 21:313-315; Slamon et al., Seminars in Oncology 2001; 28:13-19; Lidor et al., Journal of Clinical Investigation 1993; 92:2440-2447; Leopold et al., NCI Monographs 1987; 99-104; Ohta et al., Cancer Letters 2001; 162:39-48; van Moorsel et al., British Journal of Cancer 1999; 80:981-990.

Other genotoxic agents are those that form persistent genomic lesions and are preferred for use as chemotherapeutic agents in the clinical management of cancer. The rate of cellular repair of genotoxin-induced DNA damage, as well as the rate of cell growth via the cell division cycle, affects the outcome of genotoxin therapy. Unrepaired lesions in a cell's genome can impede DNA replication, impair the replication fidelity of newly synthesized DNA or hinder the expression of genes needed for cell survival. Thus, one determinant of a genotoxic agent's cytotoxicity (propensity for contributing to cell death) is the resistance of genomic lesions formed therefrom to cellular repair. Genotoxic agents that form persistent genomic lesions, e.g., lesions that remain in the genome at least until the cell commits to the cell cycle, generally are more effective cytotoxins than agents that form transient, easily repaired genomic lesions.

A general class of genotoxic compounds that are used for treating many cancers and that are affected by levels of ERCC1 expression are DNA alkylating agents and DNA intercalating agents. Psoralens are genotoxic compounds known to be useful in the photochemotherapeutic treatment of cutaneous diseases such as psoriasis, vitiligo, fungal infections and cutaneous T cell lymphoma. Harrison's Principles of Internal Medicine, Part 2 Cardinal Manifestations of Disease, Ch. 60 (12th ed. 1991). Another general class of genotoxic compounds, members of which can alkylate or intercalate into DNA, includes synthetically and naturally sourced antibiotics. Of particular interest herein are antineoplastic antibiotics, which include but are not limited to the following classes of compounds represented by: amsacrine; actinomycin A, C, D (alternatively known as dactinomycin) or F (alternatively KS4); azaserine; bleomycin; caminomycin (carubicin), daunomycin (daunorubicin), or 14-hydroxydaunomycin (adriamycin or doxorubicin); mitomycin A, B or C; mitoxantrone; plicamycin (mithramycin); and the like.

Still another general class of genotoxic agents that are commonly used and that alkylate DNA, are those that include the haloethylnitrosoureas, especially the chloroethylnitrosoureas. Representative members of this broad class include carmustine, chlorozotocin, fotemustine, lomustine, nimustine, ranimustine and streptozotocin. Haloethylnitrosourea first agents can be analogs or derivatives of any of the foregoing representative compounds.

Yet another general class of genotoxic agents, members of which alkylate DNA, includes the sulfur and nitrogen mustards. These compounds damage DNA primarily by forming covalent adducts at the N7 atom of guanine. Representative members of this broad class include chlorambucil, cyclophosphamide, ifosfamide, melphalan, mechloroethamine, novembicin, trofosfamide and the like. Oligonucleotides or analogs thereof that interact covalently or noncovalently with specific sequences in the genome of selected cells can also be used as genotoxic agents, if it is desired to select one or more predefined genomic targets as the locus of a genomic lesion. Another class of agents, members of which alkylate DNA, include the ethylenimines and methylmelamines. These classes include altretamine (hexamethylmelamine), triethylenephosphoramide (TEPA), triethylenethiophosphoramide (ThioTEPA) and triethylenemelamine, for example.

Additional classes of DNA alkylating agents include the alkyl sulfonates, represented by busulfan; the azinidines, represented by benzodepa; and others, represented by, e.g., mitoguazone, mitoxantrone and procarbazine. Each of these classes includes analogs and derivatives of the respective representative compounds.

### DPD

The present inventors disclose oligonucleotide primers and oligonucleotide primers substantially identical thereto that allow accurate assessment of DPD expression in tissues. These oligonucleotide primers, DPD3a-51F (SEQ ID NO: 5) and DPD3a-134R (SEQ ID NO: 6), (also referred to herein as the oligonucleotide primer pair DPD3A) and oligionucleotide primers DPD3b-65 IF (SEQ ID NO: 7) and DPD3b-736R (SEQ ID NO: 8), (also referred to herein as the oligonucleotide primer pair DPD3B) (See U.S. Pat. No. 7,005,278, incorporated by reference) are particularly effective when used to measure DPD gene expression in fixed paraffin embedded (FPE) tumor specimens.

This invention includes substantially identical oligonucleotides that hybridize under stringent conditions (as defined herein) to all or a portion of the oligonucleotide primer sequence of DPD3A-51F (SEQ ID NO: 5), its complement, DPD3A-134R (SEQ ID NO: 6) or its complement. Furthermore, this invention also includes substantially identical oligonucleotides that hybridize under stringent conditions (as defined herein) to all or a portion of the oligonucleotide primer sequence DPD3b-65° F. (SEQ ID NO: 7) its complement, DPD3b-736R (SEQ ID NO: 8), or its complement.

The hybridizing portion of the nucleic acids is typically at least 10 (e.g., 15) nucleotides in length. The hybridizing portion of the hybridizing nucleic acid is at least about 80%, preferably at least about 95%, or most preferably about at least 98%, identical to the sequence of a portion or all of oligonucleotide primer DPD3A-51F (SEQ ID NO: 5), its complement, DPD3A-134R (SEQ ID NO: 6) or its complement. Additionally, the hybridizing portion of the hybridizing nucleic acid is at least about 80%, preferably at least about 95%, or most preferably about at least 98%, identical to the sequence of a portion or all of oligonucleotide primer DPD3b-651F (SEQ ID NO: 7), its complement, DPD3b-736R (SEQ ID NO: 8) or its complement.

This aspect of the invention involves use of a method for reliable extraction of RNA from an FPE specimen using methods described herein and second, determination of the content of DPD mRNA in the specimen by using oligonucleotide primers oligionucleotide primer pair DPD3A (DPD3a-51F (SEQ ID NO: 5) and DPD3a-134R (SEQ ID NO: 6)) or oligonucleotides substantially identical thereto or DPD3B (DPD3b-65 IF (SEQ ID NO: 7) and DPD3b-736R (SEQ ID NO: 8)) or oligonucleotides substantially identical thereto, for carrying out reverse transcriptase polymerase chain reaction. See U.S. patent application Ser. No. 09/469,338, filed Dec. 20, 1999, and is hereby incorporated by reference.

Expression of DPD mRNA is correlated with clinical resistance to 5-FU-based chemotherapy. In particular, expression of high levels of DPD mRNA correlates with resistance to 5-FU-based chemotherapies.

The methods of this invention are applied over a wide range of tumor types. This allows for the preparation of individual “tumor expression profiles” whereby expression levels of DPD may be determined in individual patient samples and response to various chemotherapeutics can be predicted. Most preferably, the methods of the present invention are applied to bronchalveolar, small bowel or colon tumors. For application of some embodiments of the invention to particular tumor types, it is preferable to confirm the relationship of the measurement to clinical resistance by compiling a data-set of the correlation of the particular DPD expression parameter measured and clinical resistance to 5-FU-based chemotherapy.

The present methods can be applied to any type of tissue. For example, for examination of resistance of tumor tissue, it is desirable to examine the tumor tissue. Preferably, it is desirable to also examine a portion of normal tissue from the patient from which the tumor is obtained. Patients whose normal tissues are resistant to 5-FU-based chemotherapeutic compounds, but whose tumors are expected to be sensitive to such compounds, may then be treated with higher amounts of the chemotherapeutic composition.

The methods of the present invention include the step of obtaining sample of cells from a patient's tumor. Solid or lymphoid tumors, or parts thereof are surgically resected from the patient. If it is not possible to extract RNA from the tissue sample soon after its resection, the sample may then be fixed or frozen. It will then be used to obtain RNA. RNA extracted and isolated from frozen or fresh samples of resected tissue is extracted by any method known in the art, for example, Sambrook, Fischer and Maniatis, Molecular Cloning, a laboratory manual, (2nd ed.), Cold Spring Harbor Laboratory Press, New York, (1989). Preferably, care is taken to avoid degradation of RNA during the extraction process.

Alternatively, tissue obtained from the patient may be fixed, preferably by formalin (formaldehyde) or gluteraldehyde treatment, for example. Biological samples fixed by alcohol immersion are also contemplated in the present invention. Fixed biological samples are often dehydrated and embedded in paraffin or other solid supports known to those of skill in the art. Such solid supports are envisioned to be removable with organic solvents, allowing for subsequent rehydration of preserved tissue. Fixed and paraffin-embedded (FPE) tissue specimen as described herein refers to storable or archival tissue samples. RNA is extracted from the FPE cells by any of the methods described herein.

The quantification of DPD mRNA from purified total mRNA from fresh, frozen or fixed is preferably carried out using reverse-transcriptase polymerase chain reaction (RT-PCR) methods common in the art, for example. Other methods of quantifying of DPD mRNA include for example, the use of molecular beacons and other labeled probes useful in multiplex PCR. Additionally, the present invention envisages the quantification of DPD mRNA via use of a PCR-free systems employing, for example fluorescent labeled probes similar to those of the Invader® Assay (Third Wave Technologies, Inc.). Most preferably, quantification of DPD cDNA and an internal control or house keeping gene (e.g. β-actin) is done using a fluorescence based real-time detection method (ABI PRISM 7700 or 7900 Sequence Detection System [TaqMan®], Applied Biosystems, Foster City, Calif.) or similar system as described by Heid et al., (Genome Res 1996; 6:986-994) and Gibson et al. (Genome Res 1996; 6:995-1001). The output of the ABI 7700 (TaqMan® Instrument) is expressed in Ct's or “cycle thresholds”. With the TaqMan® system, a highly expressed gene having a higher number of target molecules in a sample generates a signal with fewer PCR cycles (lower Ct) than a gene of lower relative expression with fewer target molecules (higher Ct).

One aspect of the present invention resides in part in the finding that the relative amount of DPD mRNA is correlated with resistance to the chemotherapeutic agent 5-FU. It has been found herein that tumors expressing high levels of DPD mRNA are likely to be resistant to 5-FU. Conversely, those tumors expressing low amounts of DPD mRNA are likely to be sensitive to 5-FU. A patient's expression of tumor DPD mRNA is judged by comparing it to a predetermined threshold expression level of expression of DPD.

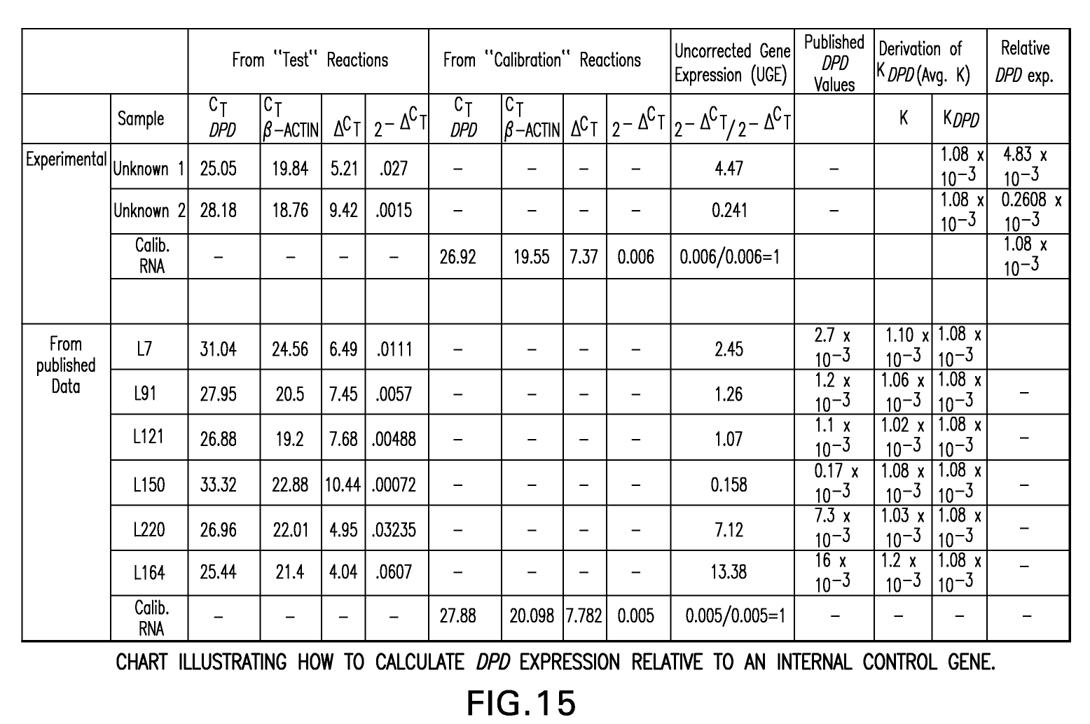

“Uncorrected Gene Expression (UGE)” as used herein as it relates to DPD refers to the numeric output of DPD expression relative to an internal control gene generated by the TaqMan® instrument. The equation used to determine UGE is shown in Example 8, and illustrated with sample calculations in FIG. 15.

A further aspect of this invention provides a method to normalize uncorrected gene expression (UGE) values acquired from the TaqMan® instrument with previously published relative gene expression values derived from non-TaqMan® technology. Preferably, the non-TaqMan® derived relative DPD:β-actin expression values previously published by Salonga, et al., Clinical Cancer Research, 6:1322-1327, 2000, hereby incorporated by reference in its entirety, are normalized with DPD UGE from a tissue sample.

“Corrected Relative DPD Expression” as used herein refers to normalized DPD expression whereby UGE is multiplied with a DPD specific correction factor (K_{DPD}), resulting in a value that can be compared to a previously published range of values. FIG. 15 illustrates these calculations in detail.

“Previously published” relative gene expression results are based on the ratio of the RT-PCR signal of a target gene to a constitutively expressed gene (β-Actin). In pre-TaqMan® technology studies, PCR reactions were run for a fixed number of cycles (i.e., 30) and endpoint values were reported for each sample. These values were then reported as a ratio of DPD expression to β-actin expression. Salonga, et al., Clinical Cancer Research, 6:1322-1327, 2000, which is hereby incorporated by reference in its entirety.

A “predetermined threshold” level of relative DPD expression, as defined herein, is a level of DPD expression above which it has been found that tumors are likely to be resistant to 5-FU. Expression levels below this threshold level are likely to be found in tumors sensitive to 5-FU. The range of relative DPD expression, among tumors responding to a 5-FU based chemotherapeutic regimen responding tumors is less than about 0.6×10⁻³ to about 2.5×10⁻³, (about a 4.2-fold range). Tumors not responding to a 5-FU based chemotherapeutic regimen have relative DPD expression of about 0.2×10⁻³ to about 16×10⁻³ (about an 80-fold range). Tumors generally do not respond to 5-FU treatment if there is a relative DPD expression greater than about 2.0×10⁻³, preferably greater than about 2.5×10⁻³. These numerical values allow the determination of whether or not the “Corrected Relative DPD Expression” of a particular sample falls above or below the “predetermined threshold” level. A threshold level of Corrected Relative DPD Expression level is about 2.0×10⁻³ to about 2.5×10⁻³.

The methods of the invention are applicable to a wide range of tissue and tumor types and so can be used for assessment of treatment in a patient and as a diagnostic or prognostic tool in a range of cancers including breast, head and neck, lung, esophageal, colorectal, and others. Preferably, the present methods are applied to prognosis of bronchoalveolar, small bowel, or colon cancer.

From the measurement of the amount of DPD mRNA that is expressed in the tumor, the skilled practitioner can make a prognosis concerning clinical resistance of a tumor to 5-FU-based chemotherapy. “5-FU-based chemotherapy” comprises administration of 5-FU, its derivatives, alone or with other chemotherapeutics, such as leucovorin or with a DPD inhibitor such as uracil, 5-ethynyluracil, bromovinyluracil, thymine, benzyloxybenzyluracil (BBU) or 5-chloro-2,4-dihydroxypyridine. Furthermore, it has been found that co-administration of a 5′-deoxy-cytidine derivative of the formula (I) with 5-FU or a derivative thereof significantly improves delivery of a chemotherapeutic agent selectively to tumor tissues as compared with the combination of 5-FU or a derivative thereof with a DPD inhibitor 5-ethynyluracil, and shows significantly improved antitumor activity in human cancer xenograft models.

### ERCC1/TS

The present invention resides in part in the finding that the amount of TS and ERCC1 mRNA is correlated with resistance to 5-FU and oxaliplatin agents, respectively. Tumors expressing high levels of TS and ERCC1 mRNA are considered likely to be resistant to platinum-based chemotherapy. Conversely, those tumors expressing low amounts of TS and ERCC1 mRNA are likely to be sensitive to platinum-based chemotherapy. A patient's tumor TS and ERCC1 mRNA expression status is judged by comparing it to a predetermined threshold expression level.

The invention provides a method of quantifying the amount of TS and ERCC1 mRNA expression in fixed or fixed and paraffin-embedded (FPE) tissue relative to gene expression of an internal control. In addition to the ERCC1 primers discussed above, the present inventors have developed oligonucleotide primers that allow accurate assessment of TS gene expression in tissues that have been fixed or fixed and embedded. The invention also provides oligonucleotide primers, TS-763F (SEQ ID NO: 9), TS-825R (SEQ ID NO: 10), or oligonucleotide primers substantially identical thereto, preferably are used together with RNA extracted from fixed and paraffin embedded (FPE) tumor samples. See U.S. Pat. No. 7,049,059, incorporated by reference. This measurement of TS and ERCC1 gene expression may then be used for prognosis of platinum-based chemotherapy.

This embodiment of the invention involves first, a method for reliable extraction of RNA from an FPE sample as described herein and second, determination of the content of TS and ERCC1 mRNA in the sample by using a pair of ERCC1 and TS oligonucleotide primers described above or oligonucleotides substantially identical thereto, for carrying out reverse transcriptase polymerase chain reaction.

The present method can be applied to any type of tissue from a patient. For examination of resistance of tumor tissue, it is preferable to examine the tumor tissue. In a preferred embodiment, a portion of normal tissue from the patient from which the tumor is obtained, is also examined. Patients whose normal tissues are expected to be resistant to platinum-based chemotherapeutic compounds, i.e., show a high level of TS and ERCC1 gene expression, but those whose tumors are expected to be sensitive to such compounds, i.e. show a low level of TS and ERCC1 gene expression, may then be treated with higher amounts of the chemotherapeutic composition.

The methods of the present invention can be applied over a wide range of tumor types. This allows for the preparation of individual “tumor expression profiles” whereby expression levels of TS and/or ERCC1 are determined in individual patient samples and response to various chemotherapeutics is predicted. Preferably, the methods of the invention are applied to solid tumors, most preferably colorectal adenocarcinoma tumors.

A “predetermined threshold level,” as defined herein relating to TS, is a level of TS expression above which it has been found that tumors are likely to be resistant to a 5-FU and 5-FU and oxaliplatin-based chemotherapeutic regimen. Expression levels below this threshold level are likely to be found in tumors sensitive to 5-FU or 5-FU and oxaliplatin-based chemotherapeutic regimen. The range of relative expression of TS, expressed as a ratio of TS:β-actin, among tumors responding to a 5-FU or 5-FU and oxaliplatin-based chemotherapeutic chemotherapeutic regimen is less than about 7.5×10⁻³. Tumors that do not respond to a 5-FU or 5-FU and oxaliplatin-based chemotherapeutic regimen have relative expression of TS:β-actin ratio above about 7.5×10⁻³.

In performing the method of the present invention ERCC1 expression levels and TS expression levels are assayed in patient tumor samples to prognosticate the efficacy of a 5-FU and oxaliplatin-based chemotherapeutic regimen. Moreover, in a method of the present invention TS expression levels are assayed in patient tumor samples to prognosticate the efficacy of a 5-FU based chemotherapeutic regimen. Additionally, in the method of the present invention ERCC1 expression levels are assayed in patient tumor samples to prognosticate the efficacy of a oxaliplatin based chemotherapeutic regimen. Alternatively, expression levels of just TS expression levels are assayed in patient tumor samples to prognosticate the efficacy of a combined 5-FU and oxaliplatin-based chemotherapeutic regimen.

In performing the method of this embodiment of the present invention, tumor cells are preferably isolated from the patient. Solid or lymphoid tumors or portions thereof are surgically resected from the patient or obtained by routine biopsy. RNA isolated from frozen or fresh samples is extracted from the cells by any of the methods typical in the art, for example, Sambrook, Fischer and Maniatis, Molecular Cloning, a laboratory manual, (2nd ed.), Cold Spring Harbor Laboratory Press, New York, (1989). Preferably, care is taken to avoid degradation of the RNA during the extraction process.

RNA is extracted from the FPE cells by any of the methods as described above. The quantification of TS and ERCC1 mRNA from purified total mRNA from fresh, frozen or fixed is preferably carried out using reverse-transcriptase polymerase chain reaction (RT-PCR) methods common in the art, for example. Other methods of quantifying of TS or ERCC1 mRNA include for example, the use of molecular beacons and other labeled probes useful in multiplex PCR. Additionally, the present invention envisages the quantification of TS and/or ERCC1 mRNA via use of a PCR-free systems employing, for example fluorescent labeled probes similar to those of the Invader® Assay (Third Wave Technologies, Inc.). Most preferably, quantification of TS and/or ERCC1 cDNA and an internal control or house keeping gene (e.g. β-actin) is done using a fluorescence based real-time detection method (ABI PRISM 7700 or 7900 Sequence Detection System [TaqMan®], Applied Biosystems, Foster City, Calif.) or similar system as described by Heid et al., (Genome Res 1996; 6:986 994) and Gibson et al. (Genome Res 1996; 6:995 1001). The output of the ABI 7700 (TaqMan® Instrument) is expressed in Ct's or “cycle thresholds”. With the TaqMan® system, a highly expressed gene having a higher number of target molecules in a sample generates a signal with fewer PCR cycles (lower Ct) than a gene of lower relative expression with fewer target molecules (higher Ct).

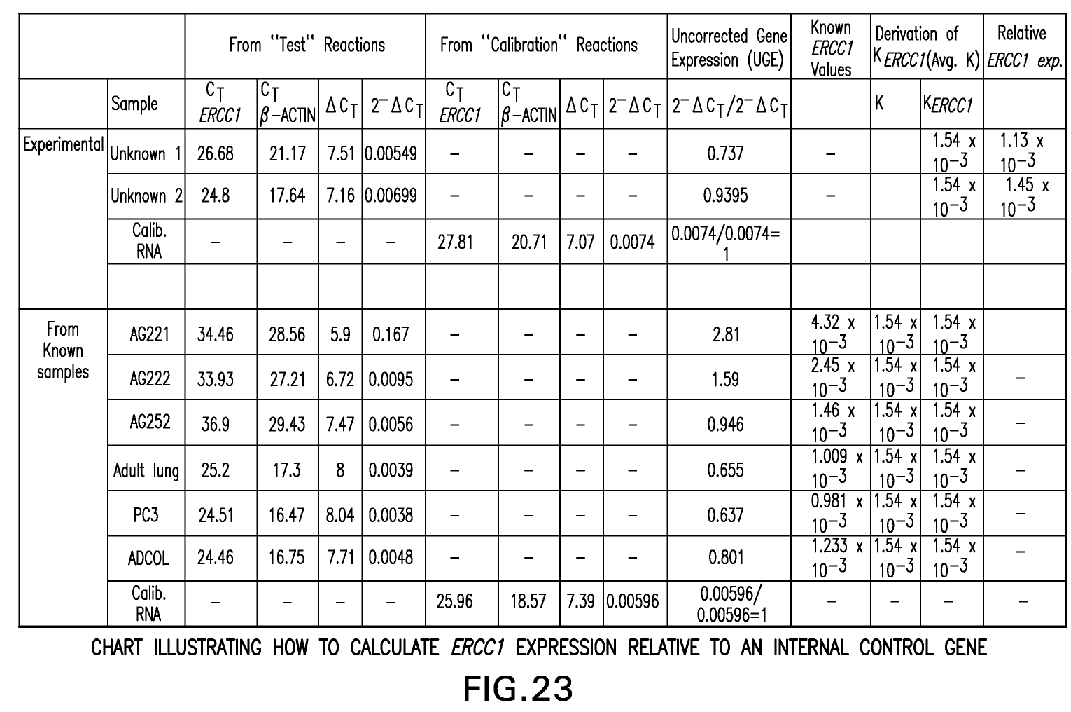

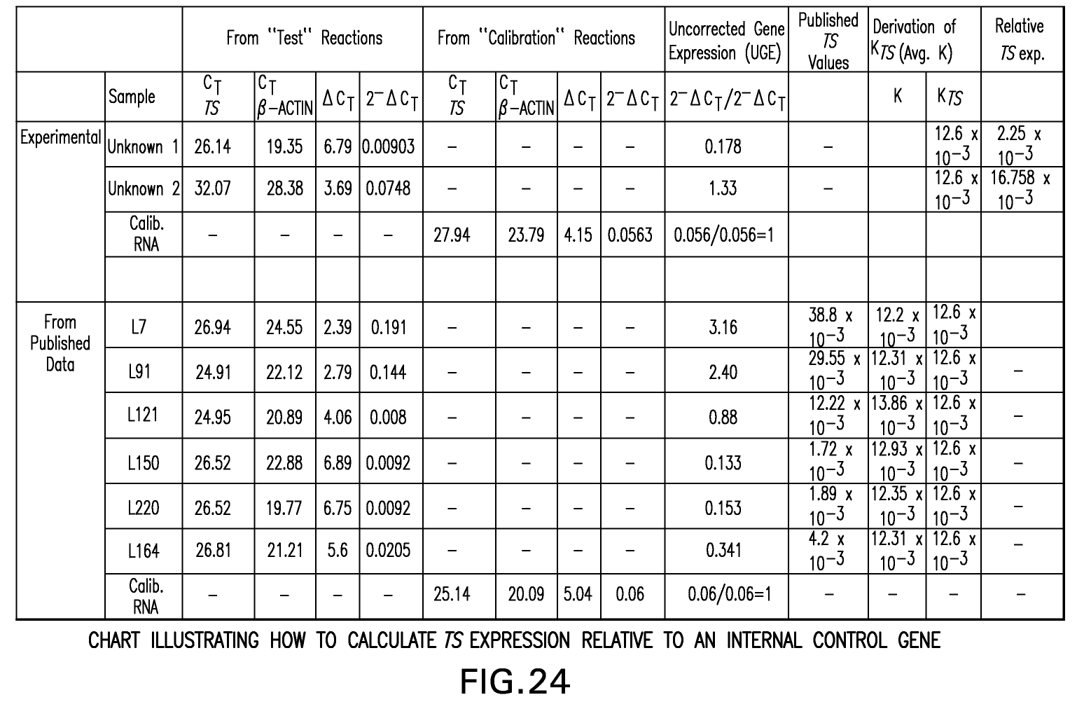

“Uncorrected Gene Expression (UGE)” as used herein refers to the numeric output of TS and/or ERCC1 expression relative to an internal control gene generated by the TaqMan® instrument. The equation used to determine UGE is shown in Examples 10 and 11, and illustrated with sample calculations in FIGS. 23 and 24.

“Corrected Relative TS Expression” as used herein refers to normalized TS expression whereby UGE is multiplied with a TS specific correction factor (K_{TS}), resulting in a value that can be compared to a known range of TS expression levels relative to an internal control gene. Example 10 and FIG. 24 illustrate these calculations in detail. These numerical values allow the determination of whether the “Corrected Relative TS Expression” of a particular sample falls above or below the “predetermined threshold” level. The predetermined threshold level of Corrected Relative TS Expression to β-actin level is about 7.5×10⁻³. K_{TS} specific for TS, the internal control β-actin and calibrator Universal PE RNA; Cat #4307281, lot #3617812014 from Applied Biosystems, is 12.6×10⁻³.

K_{TS} may be determined for an internal control gene other than β-actin and/or a calibrator RNA different than Universal PE RNA; Cat #4307281, lot #3617812014 from Applied Biosystems. To do so, one must calibrate both the internal control gene and the calibrator RNA to tissue samples for which TS expression levels relative to that particular internal control gene have already been determined (i.e., “known relative gene expression” or “previously published”). Preferably such tissue samples are formalin fixed and paraffin-embedded (FPE) samples and RNA is extracted from them according to the protocol described herein. Such a determination can be made using standard pre-TaqMan®, quantitative RT-PCR techniques well known in the art. Upon such a determination, such samples have “known relative gene expression” levels of TS useful in the determining a new K_{TS} specific for the new internal control and/or calibrator RNA as described in Example 10.

“Previously published” relative gene expression results are based on the ratio of the RT-PCR signal of a target gene to a constitutively expressed gene (β-Actin). In pre-TaqMan® technology studies, PCR reactions were run for a fixed number of cycles (i.e., 30) and endpoint values were reported for each sample. These values were then reported as a ratio of ERCC1 or TS expression to β-actin expression. Salonga, et al., Clinical Cancer Research, 6:1322 1327, 2000, incorporated herein by reference in its entirety.

The methods of the invention are applicable to a wide range of tissue and tumor types and so can be used for assessment of clinical treatment of a patient and as a diagnostic or prognostic tool for a range of cancers including breast, head and neck, lung, esophageal, colorectal, and others. In a preferred embodiment, the present methods are applied to prognosis of colorectal adenocarcinoma.

Pre-chemotherapy treatment tumor biopsies are usually available only as fixed paraffin embedded (FPE) tissues, generally containing only a very small amount of heterogeneous tissue. Such FPE samples are readily amenable to microdissection, so that TS and ERCC1 gene expression may be determined in tumor tissue uncontaminated with stromal tissue. Additionally, comparisons can be made between stromal and tumor tissue within a biopsy tissue sample, since such samples often contain both types of tissues.

Any oligonucleotide pairs that flank a region of TS gene may be used to carry out the methods of the invention. Primers hybridizing under stringent conditions to a region of the TS gene for use in the present invention will amplify a product between 20-1000 base pairs, preferably 100-400 base pairs, most preferably 200-400 base pairs.

HER2-neu/EGFR

Tumors expressing high levels of HER2-neu and/or EGFR mRNA are considered likely to be sensitive to receptor tyrosine kinase targeted chemotherapy. Conversely, those tumors expressing low amounts of HER2-neu and EGFR mRNA are not likely to be sensitive to receptor tyrosine kinase targeted chemotherapy. A patient's differential HER2-neu and EGFR mRNA expression status is judged by comparing it to a predetermined threshold expression level.

The invention provides a method of quantifying the amount of HER2-neu and/or EGFR mRNA expression in fresh, frozen, fixed or fixed and paraffin-embedded (FPE) tissue relative to gene expression of an internal control. The present inventors have developed oligonucleotide primers that allow accurate assessment of HER2-neu and EGFR gene expression in fresh, frozen, fixed or fixed and embedded tissues. The oligonucleotide primers, EGFR-1753F (SEQ ID NO: 11), EGFR-1823R (SEQ ID NO: 12), or oligonucleotide primers substantially identical thereto, preferably are used together with RNA extracted from fresh, frozen, fixed or fixed and paraffin embedded (FPE) tumor samples. The invention also provides oligonucleotide primers, HER2-neu 2671F (SEQ ID NO: 13), HER2-neu 2699R (SEQ ID NO: 14)(See U.S. Pat. No. 6,582,919 incorporated by reference), or oligonucleotide primers substantially identical thereto, preferably are used together with RNA extracted from fresh, frozen, fixed or fixed and paraffin embedded (FPE) tumor samples. This measurement of HER2-neu and/or EGFR gene expression may then be used for prognosis of receptor tyrosine kinase targeted chemotherapy

This embodiment of the invention involves, a method for reliable extraction of RNA from fresh, frozen, fixed or FPE samples, and determination of the content of EGFR mRNA in the sample using the methods described herein and by using a pair of oligonucleotide primers, preferably oligionucleotide primer pair EGFR-1753F (SEQ ID NO: 11) and EGFR-1823R (SEQ ID NO: 12), or oligonucleotides substantially identical thereto, for carrying out reverse transcriptase polymerase chain reaction.

Another embodiment of the invention involves a method for reliable extraction of RNA from fresh, frozen, fixed or FPE samples, and determination of the content of HER2-neu mRNA in the sample by using methods described herein and using a pair of oligonucleotide primers oligonucleotide primers, HER2-neu 2671F (SEQ ID NO: 13), HER2-neu 2699R (SEQ ID NO: 14), or oligonucleotide primers substantially identical thereto.

The methods of the present invention can be applied over a wide range of tumor types. This allows for the preparation of individual “tumor expression profiles” whereby expression levels of HER2-neu and/or EGFR are determined in individual patient samples and response to various chemotherapeutics is predicted. Preferably, the methods of the invention are applied to solid tumors, most preferably NSCLC tumors.

A “differential expression level” as defined herein refers to the difference in the level of expression of either EGFR or HER2-neu in a tumor with respect to the level of expression of either EGFR or HER2-neu in a matching non-malignant tissue sample, respectively. The differential expression level is determined by dividing the UGE of a particular gene from the tumor sample with the UGE of the same gene from a matching non-malignant tissue sample.

A “predetermined threshold level”, as defined herein relating to EGFR expression, is a level of differential EGFR expression above which (i.e., high), tumors are likely to be sensitive to a receptor tyrosine kinase targeted chemotherapeutic regimen. A high differential EGFR expression level is prognostic of lower patient survivability. Tumors with expression levels below this threshold level are not likely to be affected by a receptor tyrosine kinase targeted chemotherapeutic regimen. A low differential EGFR expression level is prognostic of higher patient survivability. Whether or not differential expression is above or below a “predetermined threshold level” is determined by the method used by Mafune et al., who calculated individual differential tumor/normal (T/N) expression ratios in matching non-malignant tissues obtained from patients with squamous cell carcinoma of the esophagus. Mafune et al., Clin Cancer Res 5:4073-4078, 1999. This method of analysis leads to a precise expression value for each patient, being based on the individual background expression obtained from matching non-malignant tissue. The differential expression of EGFR is considered “high” and indicative of low survivability if the UGE of EGFR:β-actin in a tumor sample divided by the UGE of EGFR:β-actin in a matching non-malignant tissue sample, is above the predetermined threshold value of about 1.8. The differential expression of EGFR is considered “low” and indicative of high survivability if the UGE of EGFR:β-actin in a tumor sample divided by the UGE of EGFR:β-actin in a matching n+on-malignant tissue sample, is below the predetermined threshold value of about 1.8.

A “predetermined threshold level,” as defined herein relating to differential HER2-neu expression, is a level of HER2-neu expression above which (i.e., high), tumors are likely to be sensitive to a receptor tyrosine kinase targeted chemotherapeutic regimen. A high differential HER2-neu expression level is prognostic of lower patient survivability. Tumors with expression levels below this threshold level are not likely to be affected by a receptor tyrosine kinase targeted chemotherapeutic regimen. A low differential HER2-neu expression level is prognostic of higher patient survivability. The differential expression of HER2-neu is considered “high” and indicative of low survivability if the UGE of HER2-neu:β-actin in a tumor sample divided by the UGE of HER2-neu:β-actin in a matching non-malignant tissue sample, is above the predetermined threshold value of about 1.8. The differential expression of HER2-neu is considered “low” and indicative of high survivability if the UGE of HER2-neu:β-actin in a tumor sample divided by the UGE of HER2-neu:β-actin in a matching non-malignant tissue sample, is below the predetermined threshold value of about 1.8.

A “threshold level” for HER2-neu was determined using the following results and method. The corrected HER2-neu mRNA expression, expressed as the ratio between HER2-neu and β-Actin PCR product, was 4.17×10⁻³ (range 0.28-23.86×10³) in normal lung and 4.35×10⁻³ (range: 0.21-68.11×10⁻³) in tumor tissue (P=0.019 Wilcoxon test). The maximal chi-square method by Miller and Siegmund (Miller et al., Biometrics 38:1011-1016, 1982) and Halpern (Biometrics 38:1017-1023, 1982) determined a threshold value of 1.8 to segregate patients into low and high differential HER2-neu expressors. By this criterion, 29 (34.9%) patients had a high differential HER2-neu expression and 54 (65.1%) had a low differential HER2-neu expression.

A “threshold level” for EGFR was determined using the following results and method. The median corrected EGFR mRNA expression was 8.17×10⁻³ (range: 0.31-46.26×10⁻³) in normal lung and 7.22×10⁻³ (range: 0.27-97.49×10⁻³) in tumor tissue (P=n.s.). The maximal chi-square method (Miller (1982); Halpern (1982)) determined a threshold value of 1.8 to segregate patients into low and high differential EGFR expressors. By this criterion, 28 (33.7%) patients had a high differential EGFR expression and 55 (66.3%) had a low differential EGFR expression status.

In performing the method of the present invention either differential EGFR expression levels or differential HER2-neu expression levels are assayed in a patient to prognosticate the efficacy of a receptor tyrosine kinase targeted chemotherapeutic regimen. Moreover, in the method of the present invention differential HER2-neu expression levels are assayed in a patient prognosticate the efficacy of a receptor tyrosine kinase targeted chemotherapeutic regimen. Additionally, in the method of the present invention differential EGFR expression levels are assayed in a patient to prognosticate the efficacy of a receptor tyrosine kinase targeted chemotherapeutic regimen. Alternatively, both differential EGFR expression levels and differential HER2-neu expression levels are assayed in a patient to prognosticate the efficacy of a receptor tyrosine kinase targeted chemotherapeutic regimen.

“Matching non-malignant sample” as defined herein refers to a sample of non-cancerous tissue derived from the same individual as the tumor sample to be analyzed for differential EGFR and/or differential HER2-neu expression. Preferably a matching non-malignant sample is derived from the same organ as the organ from which the tumor sample is derived. Most preferably, the matching non-malignant tumor sample is derived from the same organ tissue layer from which the tumor sample is derived. Also, it is preferable to take a matching non-malignant tissue sample at the same time a tumor sample is biopsied. In a preferred embodiment tissues from the following two locations are analyzed: lung tumor and non-malignant lung tissue taken from the greatest distance form the tumor or colon tumor and non-malignant colon tissue taken from the greatest distance form the tumor as possible under the circumstances.

In performing the method of this embodiment of the present invention, tumor cells are preferably isolated from the patient. Solid or lymphoid tumors or portions thereof are surgically resected from the patient or obtained by routine biopsy. RNA isolated from frozen or fresh tumor samples is extracted from the cells by any of the methods typical in the art, for example, Sambrook, Fischer and Maniatis, Molecular Cloning, a laboratory manual, (2nd ed.), Cold Spring Harbor Laboratory Press, New York, (1989). Preferably, care is taken to avoid degradation of the RNA during the extraction process.

However, tissue obtained from the patient after biopsy is often fixed, usually by formalin (formaldehyde) or gluteraldehyde, for example, or by alcohol immersion. Fixed biological samples are often dehydrated and embedded in paraffin or other solid supports known to those of skill in the art. See Plenat et al., Ann Pathol January 2001; 21(1):29-47. Non-embedded, fixed tissue as well as fixed and embedded tissue may also be used in the present methods. Solid supports for embedding fixed tissue are envisioned to be removable with organic solvents for example, allowing for subsequent rehydration of preserved tissue.

RNA is extracted from paraffin-embedded (FPE) tissue cells by any of the methods described herein. The quantification of HER2-neu or EGFR mRNA from purified total mRNA from fresh, frozen or fixed is preferably carried out using reverse-transcriptase polymerase chain reaction (RT-PCR) methods common in the art, for example. Other methods of quantifying of HER2-neu or EGFR mRNA include for example, the use of molecular beacons and other labeled probes useful in multiplex PCR. Additionally, the present invention envisages the quantification of HER2-neu and/or EGFR mRNA via use of a PCR-free systems employing, for example fluorescent labeled probes similar to those of the Invader® Assay (Third Wave Technologies, Inc.). Most preferably, quantification of HER2-neu and/or EGFR cDNA and an internal control or house keeping gene (e.g. β-actin) is done using a fluorescence based real-time detection method (ABI PRISM 7700 or 7900 Sequence Detection System [TaqMan®], Applied Biosystems, Foster City, Calif.) or similar system as described by Heid et al., (Genome Res 1996; 6:986-994) and Gibson et al. (Genome Res 1996; 6:995-1001). The output of the ABI 7700 (TaqMan® Instrument) is expressed in Ct's or “cycle thresholds.” With the TaqMan® system, a highly expressed gene having a higher number of target molecules in a sample generates a signal with fewer PCR cycles (lower Ct) than a gene of lower relative expression with fewer target molecules (higher Ct).

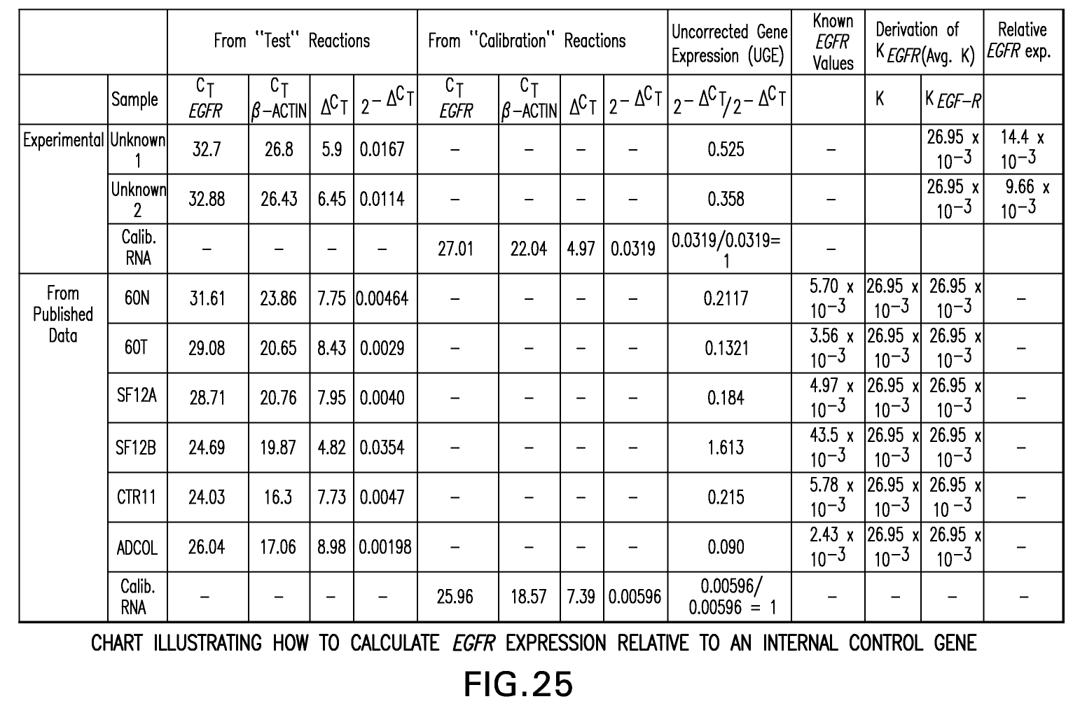

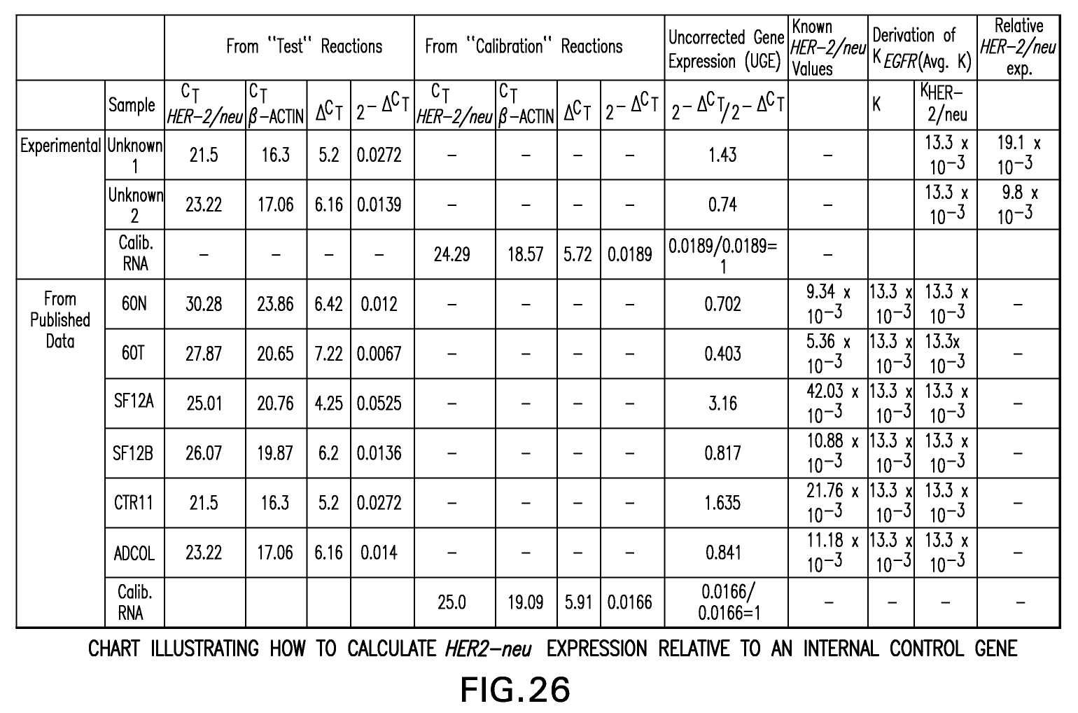

“Uncorrected Gene Expression (UGE)” as used herein refers to the numeric output of HER2-neu and/or EGFR expression relative to an internal control gene generated by the TaqMan® instrument. The equation used to determine UGE is shown in Examples 12 and 13, and illustrated with sample calculations in FIGS. 25 and 26.

These numerical values allow the determination of whether or not the differential gene expression (i.e., “UGE” or of a particular tumor sample divided by the “UGE” of a matching non-tumor sample) falls above or below the “predetermined threshold” level. The predetermined threshold level for EGFR and HER2-neu is about 1.8.

A further aspect of this invention provides a method to normalize uncorrected gene expression (UGE) values acquired from the TaqMan® instrument with “known relative gene expression” values derived from non-TaqMan® technology. Preferably, TaqMan® derived HER2-neu and/or EGFR UGE values from a tissue sample are normalized to samples with known non-TaqMan® derived relative HER2-neu and/or EGFR:β-actin expression values.

“Corrected Relative EGFR Expression” as used herein refers to normalized EGFR expression whereby UGE is multiplied with a EGFR specific correction factor (K_{EGFR}), resulting in a value that can be compared to a known range of EGFR expression levels relative to an internal control gene. Example 12 and FIG. 25 illustrate these calculations in detail. K_{EGFR} specific for EGFR, the internal control β-actin and calibrator Human Liver Total RNA (Stratagene, Cat #735017), is 26.95×10⁻³. These numerical values also allow the determination of whether or not the “Corrected Relative Expression” of a particular tumor sample divided by the “Corrected Relative Expression” of a matching non-tumor sample (i.e., differential expression) falls above or below the “predetermined threshold” level. The predetermined threshold level for HER2-neu or EGFR is about 1.8. In determining whether the differential expression of either EGFR or HER2-neu in a tumor sample is 1.8 times greater than in a matching non-tumor sample, one will readily recognize that either UGE values or Corrected Relative Expression values can be used. For example, if one divides the Corrected Relative Expression level of the tumor with that of the matching non-tumor sample, the K-factor cancels out and one is left with same ratio as if one had used UGE values.

“Known relative gene expression” values are derived from previously analyzed tissue samples and are based on the ratio of the RT-PCR signal of a target gene to a constitutively expressed internal control gene (e.g. β-Actin, GAPDH, etc.). Preferably such tissue samples are formalin fixed and paraffin-embedded (FPE) samples and RNA is extracted from them according to the protocol described herein. To quantify gene expression relative to an internal control standard quantitative RT-PCR technology known in the art is used. Pre-TaqMan® technology PCR reactions are run for a fixed number of cycles (i.e., 30) and endpoint values are reported for each sample. These values are then reported as a ratio of EGFR expression to β-actin expression.

K_{EGFR} may be determined for an internal control gene other than β-actin and/or a calibrator RNA different than Human Liver Total RNA (Stratagene, Cat #735017). To do so, one must calibrate both the internal control gene and the calibrator RNA to tissue samples for which EGFR expression levels relative to that particular internal control gene have already been determined (i.e., “known relative gene expression”). Preferably such tissue samples are formalin fixed and paraffin-embedded (FPE) samples and RNA is extracted from them according to the protocol described in Example 1. Such a determination can be made using standard pre-TaqMan®, quantitative RT-PCR techniques well known in the art. Upon such a determination, such samples have “known relative gene expression” levels of EGFR useful in the determining a new K_{EGFR} specific for the new internal control and/or calibrator RNA as described in Example 12.

“Corrected Relative HER2-neu Expression” as used herein refers to normalized HER2-neu expression whereby UGE is multiplied with a HER-neu specific correction factor (K_{HER2-neu}), resulting in a value that can be compared to a known range of HER2-neu expression levels relative to an internal control gene. Example 13 and FIG. 26 illustrate these calculations in detail. K_{HER-neu} specific for HER2-neu, the internal control β-actin and calibrator Human Liver Total RNA (Stratagene, Cat #735017), is 13.3×10⁻³.

K_{HER2-neu} may be determined for an internal control gene other than β-actin and/or a calibrator RNA different than Human Liver Total RNA (Stratagene, Cat #735017). To do so, one must calibrate both the internal control gene and the calibrator RNA to tissue samples for which HER2-neu expression levels relative to that particular internal control gene have already been determined (i.e., “known relative gene expression”). Preferably such tissue samples are formalin fixed and paraffin-embedded (FPE) samples and RNA is extracted from them according to the protocol described in herein. Such a determination can be made using standard pre-TaqMan®, quantitative RT-PCR techniques well known in the art, for example. Upon such a determination, such samples have “known relative gene expression” levels of HER-neu useful in the determining a new K_{HER2-neu} specific for the new internal control and/or calibrator RNA as described in Example 13.

The methods of the invention are applicable to a wide range of tissue and tumor types and so can be used for assessment of clinical treatment of a patient and as a diagnostic or prognostic tool for a range of cancers including breast, head and neck, lung, esophageal, colorectal, and others. In a preferred embodiment, the present methods are applied to prognosis of NSCLC tumors.

Pre-chemotherapy treatment tumor biopsies are usually available only as fixed paraffin embedded (FPE) tissues, generally containing only a very small amount of heterogeneous tissue. Such FPE samples are readily amenable to microdissection, so that HER2-neu and/or EGFR gene expression may be determined in tumor tissue uncontaminated with non-malignant stromal tissue. Additionally, comparisons can be made between non-malignant stromal and tumor tissue within a biopsy tissue sample, since such samples often contain both types of tissues.

Generally, any oligonucleotide pairs that flank a region of EGFR gene, as shown in SEQ ID NO: 10, may be used to carry out the methods of the invention. Primers hybridizing under stringent conditions to a region of the EGFR gene for use in the present invention will amplify a product between 20-1000 base pairs, preferably 100-400 base pairs, most preferably 200-400 base pairs.

Furthermore, any oligonucleotide pairs that flank a region of HER2-neu gene, may be used to carry out the methods of the invention. Primers hybridizing under stringent conditions to a region of the HER2-neu gene for use in the present invention will amplify a product between about 20-1000 base pairs, preferably 100-400 base pairs, most preferably 200-400 base pairs.

Over-activity of HER2-neu refers to either an amplification of the gene encoding HER2-neu or the production of a level of HER2-neu activity which can be correlated with a cell proliferative disorder (i.e., as the level of HER2-neu increases the severity of one or more of the symptoms of the cell proliferative disorder increases).

The invention being thus described, practice of the invention is illustrated by the experimental examples provided below. The skilled practitioner will realize that the materials and methods used in the illustrative examples can be modified in various ways. Such modifications are considered to fall within the scope of the present invention.

### EXAMPLES

### Example 1

### Long-Fragment RNA Extraction Procedure

I. Tissue Preparation Standard laboratory procedures are used to mount a 10 micron section of a paraffin block containing a FFPE tissue on a glass slide without a cover slip. For deparaffinization and nuclear fast red (NFR) staining the slides are treated as follows:

The slides are washed twice in Xylene for 5 minutes, followed by ethanol (“EtoH”) washes. The slide is stained with NFR using standard laboratory procedures.

Areas of interest (e.g., tumor tissue or stromal tissue) are excised either manually or with a laser capture microdissector (depending on the size of the area to be excised).

II. RNA Extraction

An extraction solution is prepared containing Tris/HCL, EDTA, SDS and water. A tumor tissue is added to the extraction solution in a centrifuge tube and proteinase K. The sample is then heated at the appropriate temperature and time for the maximal yield of long fragment RNA. For example, the sample is heated at 50° C. for about 16 hours. After the heating step, the sample is transferred to a larger tube and 2M sodium acetate (NaOAC) is added. A phenol/chloroform/isoamyl alcohol (PCI) extraction is performed. The upper aqueous phase is transferred to a new clean tube and glycogen is added. The RNA is precipitated with isopropanol (iPrOH). The pelleted RNA is mixed with a chaotropic agent (such as 0.5% sarcosine-guanidine isothiocyanate (GITC)). Dithiothreitol (DTT) is also added to the tube. 5 mM Tris is added and mixed. Then, 2M NaOAc and PCI is added, vortexed, and the tube is incubated on ice. The tube is spun and the upper aqueous phase is transferred to a new tube containing glycogen. The RNA is again pelleted (using iPrOH) and ethanol washed. The RNA is suspended in 5 mM Tris.

III. PCR Quantitation

Using extracted RNA obtained by methods of the present invention, cDNA preparation (35) and real time RT-PCR quantification are performed as previously described (36,37). PCR's of each extraction are done in triplicate. The data are reported as Ct values of the β-actin gene. The Ct value designates the amount of PCR product and, therefore, related to the original amount of target present in the PCR reaction. The relation is an inverse one, i.e., a larger Ct number indicates less target cDNA originally present. Each PCR cycle indicates a two-fold difference in amount so that, for example, a 4-cycle Ct difference between two PCR reactions means a 16-fold difference in cDNA content (2⁴=16).

### Example 2

### Method of Determining Length Distribution of Isolated RNA

To determine the relative amounts of various RNA fragment lengths isolated from FFPE tissues, the following strategy was used. RNA isolated from the FFPE specimens using the present invention and other known extraction methods was converted to cDNA using oligo dT primers. This means that only mRNA fragments containing a 3′-oligo A tail would be extended and converted to cDNA, thus providing a starting point from which to measure fragment length. PCR amplification of β-actin mRNA was used to represent the total population of mRNA. Primers were chosen to amplify approximately 100-120 bp segments of the β-actin gene representing locations 100, 300, 400 and 1000 bp from the 3′-end of the mRNA (FIG. 2). With this strategy, any difference in length-dependent efficiency of amplification would be minimized, as opposed to actually trying to amplify 100, 300, 400 and 1000 bp fragments. Thus the Ct of the PCR products of each of the primer sets should represent nearly the real ratio of the quantities of each fragment size.

TABLE 1
Strategy for determining the length of RNA fragments isolated
from FFPE tissue by amplifying segments of β-actin cDNA
located about 100-300, 400 and 1000 bp from the 3′-end of the
coding region.

Amplicon

| | | |
|---|---|---|
| 100 bp | 21-105 | from 3′ end |
| 300 bp | 206-293 | from 3′ end |
| 400 bp | 322-407 | from 3′ end |
| 1000 bp | 1050-1110 | from 3′ end |

### Example 3

### Effects of Proteinase K

This example illustrates the effect of proteinase K concentration on RNA yield and DNA contamination. The proteinase K concentration was varied over a 4-fold range (5-20 μg, designated as 1×-4× in the figure) at incubation times of 0.5, 2, 3 and 16 hr. at 50° C. As seen in FIG. 4, 1× (5 μg) of proteinase K gives about a 2-fold (1 Ct) better RNA yield than higher amounts but more importantly, amounts of proteinase K greater than 1× give appreciably higher DNA contamination (2-3 Ct cycles). This experiment also illustrates the influence of incubation time on the amount of DNA extracted, which is from 3 to 7 Ct cycles greater at the 16 hr incubation time than at the shorter incubation times. DNA is detected in the extracts by performing the PCR without first doing a reverse transcription reaction to convert RNA to cDNA (the “no reverse transcription or NRT control”). This way, the only PCR amplification that occurs is of the co-extracted DNA, which if too high can give a high background value in the PCR quantitation of the RNA and thus lead to unreliable data.

### Example 4

### Minimizing Co-Extraction of DNA

This example illustrates a procedure to selectively remove DNA from the FFPE extracts with minimal loss of RNA. In an effort to remove more DNA, an experiment was performed to test the effectiveness of a second phenol/chloroform extraction procedure that included the chaotrope, GITC. The following extraction methods were compared for RNA yield and DNA contamination:

1. Incubation of the FFPE tissue at 92° C. for 30 min and phenol/chloroform/isoamyl (“PCI”) extraction (the “RGI” method) or also referred to as “the high temperature chaotrope method.” This is a rapid short-incubation-high temperature method that was previously developed for extracting RNA from FFPE for high-throughput RT-PCR quantitation of gene expression. This method, which is used here for comparison purposes, is described in U.S. Pat. No. 6,248,535 and involves one extraction with PCI followed by isopropanal (“iPrOH”) precipitation and ethanol (“EtOH”) wash. One embodiment of the present invention was compared with the RGI method and this is designated as “PK.”

2 PK (50° C., 16 hr with proteinase K)+PCI+iPrOH+EtOH (i.e., single phenol/chloroform extraction);

3. PK+GITC and PCI+iPrOH+EtOH (adding GITC in the single phenol/chloroform extraction procedure);

4. PK+PCI+iPrOH+EtOH+GITC+PCI+iPrOH+EtOH (a double phenol/chloroform extraction with GITC included in the second phenol/chloroform);

5. PK+PCI+iPrOH+EtOH+add Tris_+PCI+iPrOH+EtOH (a double phenol/chloroform extraction with Tris in the second phenol/chloroform instead of GITC).

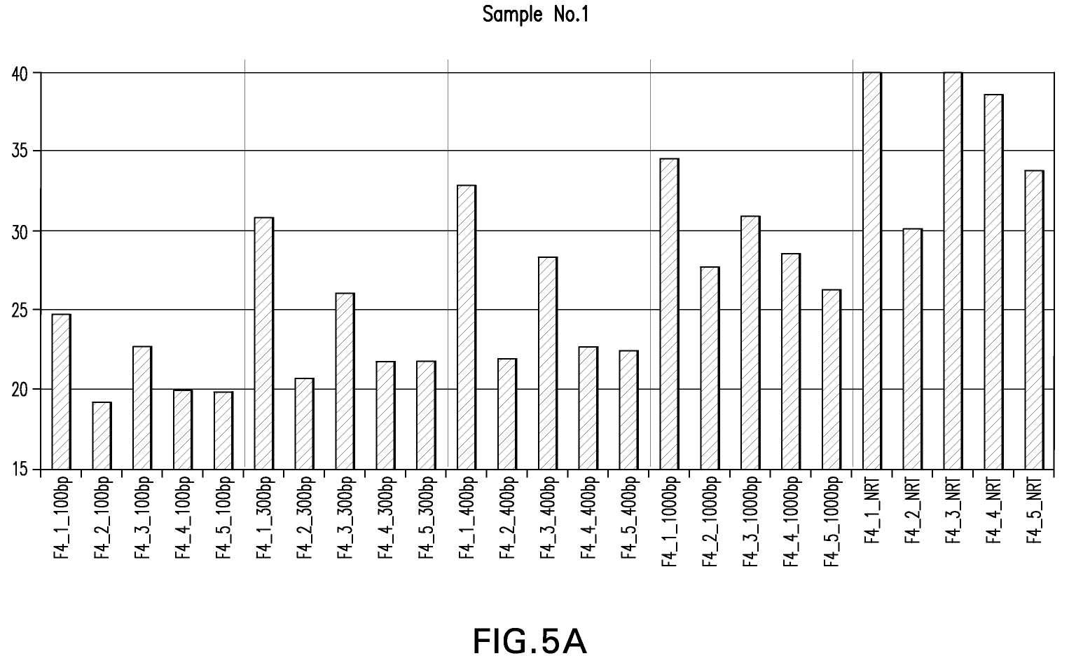

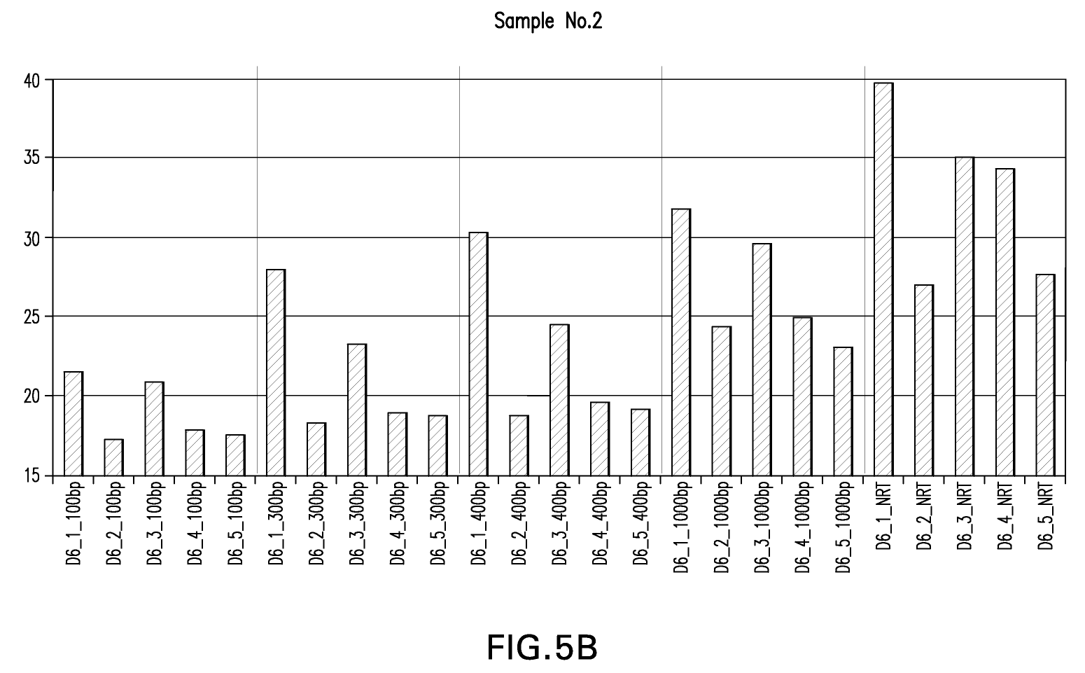

FIG. 5 shows the results of these experiments. The high-temperature (RGI) method gives the least DNA contamination because of the short incubation time but also a low yield of RNA (first bar of each series). The long-incubation PK method gives more RNA but has high DNA contamination (second bar). The effect of adding GITC in the first extraction step resulted in less DNA, but also there was also a decrease in the yield of RNA (third bar). The effect of using GITC in a second phenol/chloroform extraction step was only a slightly less RNA yield than a single phenol/chloroform extraction (about 1 Ct cycle) (fourth bar). However, there was also a decrease of DNA by 7 Ct cycles compared to the single phenol/chloroform extraction (fourth bar of the NRT series). When Tris was used instead of GITC in the second phenol/chloroform extraction, the yield of RNA remained the same but DNA was decreased by only 3 Ct cycles (fifth bar), illustrating the desirability for the chaotrope in the second phenol/chloroform extraction step. From these results, it is concluded that a second phenol/chloroform extraction step containing the chaotrope GITC is the most effective in terms of RNA yield and lowest DNA contamination.

### Example 5

### Comparison of RNA Extracted by PK Extraction Method with RNA Extracted by Two Other Extraction Methods

This example evaluates the RNA extracted by a method of the present invention (the “PK” method) by several different criteria.

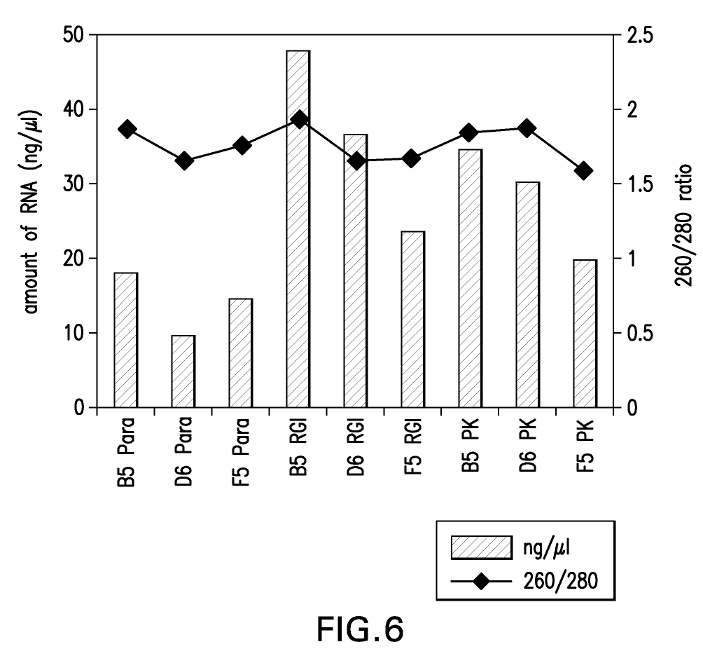

FIG. 6 shows spectrophotometric quantitation of the total amount of RNA isolated from tumor samples B5, D6 and F5 by the PK method, the RGI method (discussed above) and the Paradise kit (Arcturus, Co., Mountain View, Calif.), which is a commercially available method for isolation of RNA from FFPE samples utilizing a column purification step. As indicated by the higher UV absorbance, a PK method gave a higher yield of total RNA than the Paradise kit in the 3 samples tested, but not as high of a yield of total RNA as the RGI method. The 260/280 absorbance ratio, which indicates the purity of the RNA, was close to 1.8 for 2/3 samples isolated by the PK method (the ratio for pure RNA is 1.8).

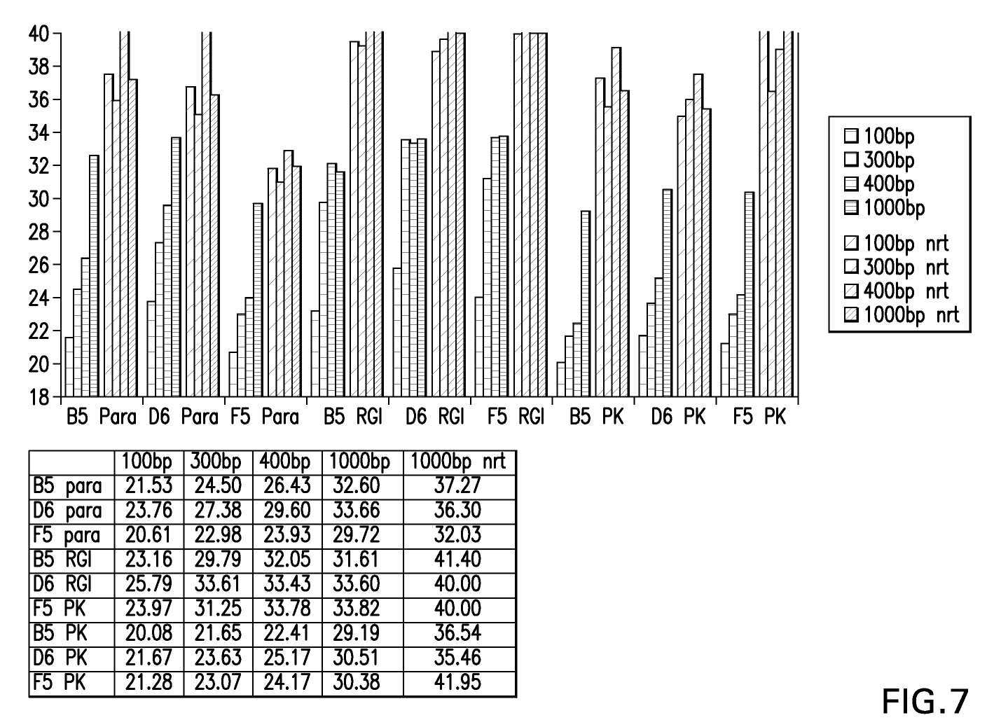

FIG. 7 compares the amounts of 100, 300, 400 and 1000 bp RNA fragments isolated by a PK method, the RGI method and the Paradise kit in tumor samples F5, D5 and D6. The quantitative amount of each fragment was determined by PCR amplification. The data show that a PK method gives the optimal results, both in terms of RNA yield and DNA contamination. The apparent discrepancy between the apparently greater yield of RNA from the RGI method suggested by higher the UV absorbance in FIG. 7 and the lower yield indicated by the PCR in this experiment occurs presumably because the high-temperature method yields many very short fragments that contribute to the overall optical absorbance at 260 nm but cannot be amplified by the primer-probe sets of the PCR due to their short length.

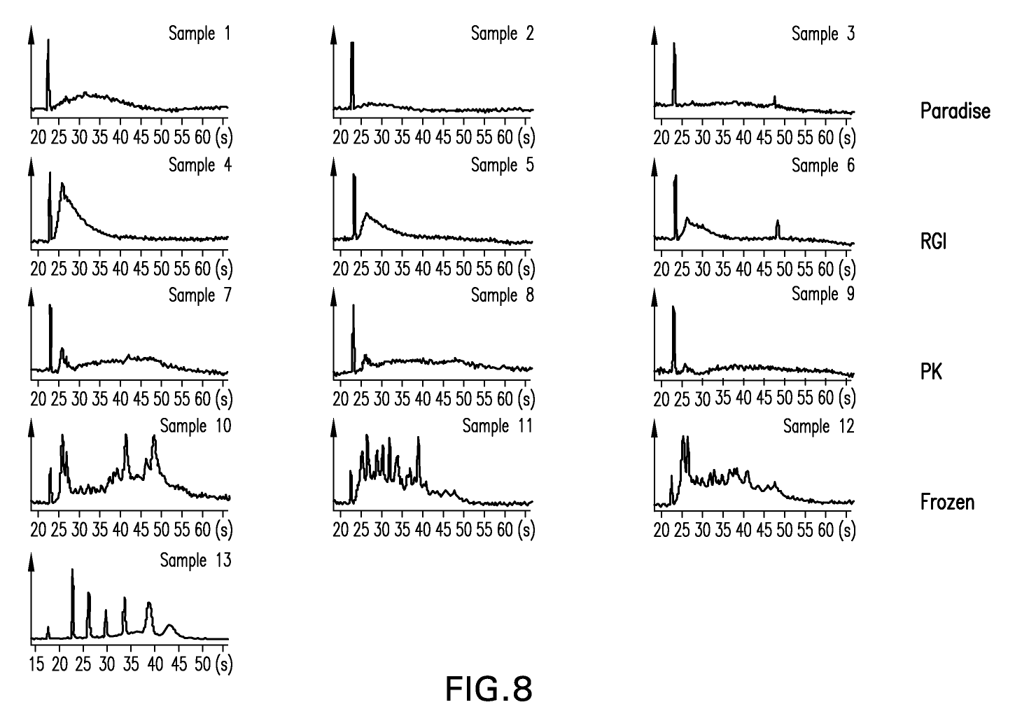

FIG. 8 compares the size distributions of the RNA fragments isolated by the PK method, the RGI method and the Paradise kit. RNA extracted by the three methods was analyzed on an Agilent 2100 Bioanalyzer (Agilent Technologies, Palo Alto, Calif.) using the RNA 6000 Nano Assay according to the manufacturer's instructions and using the Agilent 2100 Bioanalyzer Software. This analyzer separates the oligonucleic acid molecules by elution time on a size-exclusion column with shorter length RNA coming off sooner and thus located closer to the y-axis in these plots. The RGI method gave predominantly short fragments while RNA isolated by the present method (PK method) gave a range of fragment sizes with a higher yield of longer fragments than the Paradise method.

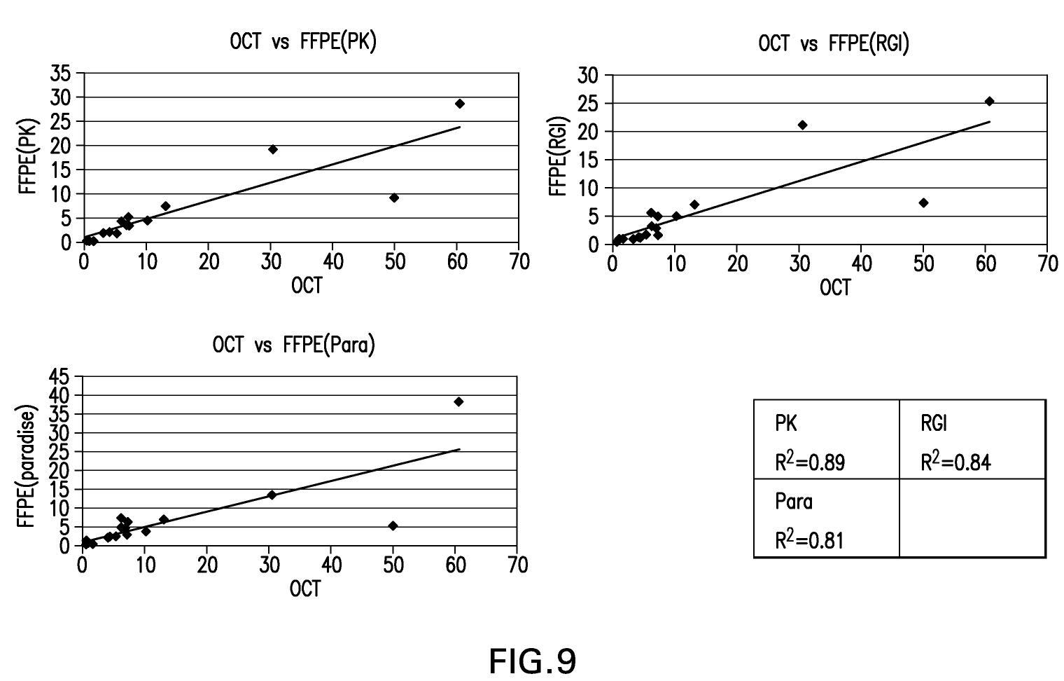

FIG. 9 shows a comparison of the gene expression of β-actin in RNA isolated from FFPE tissue using the present (PK) method to that isolated using the conventional acid guanidium thiocyanate phenol chloroform (AGPC) method (Chomczynski and Saachi, Anal Biochem (1987) 162:156-159) from matched sets of fresh frozen tissue. An excellent correlation (R=0.89) of β-actin gene expression was obtained with RNA isolated from fresh-frozen and FFPE matched specimen sets.

### Example 6

### Determining the Uncorrected Gene Expression (UGE) for ERCC1

Two pairs of parallel reactions are carried out, i.e., “test” reactions and the “calibration” reactions. The ERCC1 amplification reaction and the β-actin internal control amplification reaction are the test reactions. Separate ERCC1 and β-actin amplification reactions are performed on the calibrator RNA template and are referred to as the calibration reactions. The TaqMan® instrument will yield four different cycle threshold (Ct) values: Ct_{ERCC1} and Ct_{β-actin} from the test reactions and Ct_{ERCC1} and Ct_{β-actin} from the calibration reactions. The differences in Ct values for the two reactions are determined according to the following equation:

ΔCtₜₑₛₜ=Ct_{ERCC1}−Ct_{β-actin} (From the “test” reaction)

ΔCt_{calibrator}=Ct_{ERCC1}−Ct_{β-actin} (From the “calibration” reaction)

Next the step involves raising the number 2 to the negative ΔCt, according to the following equations.

2^{-ΔCt}ₜₑₛₜ (From the “test” reaction)

2^{-ΔCt}_{calibrator} (From the “calibration” reaction)

In order to then obtain an uncorrected gene expression for ERCC1 from the TaqMan® instrument the following calculation is carried out.

Uncorrected gene expression (UGE) for ERCC1=2^{-ΔCt}ₜₑₛₜ/^{-ΔCt}_{calibrator}

Normalizing UGE with Known Relative ERCC1 Expression Levels

The normalization calculation entails a multiplication of the UGE with a correction factor (K_{ERCC1}) specific to ERCC1 and a particular calibrator RNA. A correction factor K_{ERCC1} can also be determined for any internal control gene and any accurately pre-quantified calibrator RNA. Preferably, the internal control gene β-actin and the accurately pre-quantified calibrator RNA Human Liver Total RNA (Stratagene, Cat. #735017), are used. Given these reagents correction factor K_{ERCC1} equals 1.54×10⁻³.

Normalization is accomplished using a modification of the Δ Ct method described by Applied Biosystems, the TaqMan® manufacturer, in User Bulletin #2 and described above. To carry out this procedure, the UGE of 6 different test tissues was analyzed for ERCC1 expression using the TaqMan® methodology described above. The internal control gene β-actin and the calibrator RNA, Human Liver Total RNA (Stratagene, Cat. #735017) was used.

The known relative ERCC1 expression level of each sample AG221, AG222, AG252, Adult Lung, PC3, AdCol was divided by its corresponding TaqMan® derived UGE to yield an unaveraged correction factor K.

K_{unaveraged}=Known Values/UGE

Next, all of the K values are averaged to determine a single K_{ERCC1} correction factor specific for ERCC1, Human Liver Total RNA (Stratagene, Cat. #735017) from calibrator RNA and β-actin.

Therefore, to determine the Corrected Relative ERCC1 Expression in an unknown tissue sample on a scale that is consistent with pre-TaqMan®ERCC1 expression studies, one merely multiplies the uncorrected gene expression data (UGE) derived from the TaqMan® apparatus with the K_{ERCC1} specific correction factor, given the use of the same internal control gene and calibrator RNA.

Corrected Relative ERCC1 Expression=UGE×K_{ERCC1}

A K_{ERCC1} may be determined using any accurately pre-quantified calibrator RNA or internal control gene. Future sources of accurately pre-quantified RNA can be calibrated to samples with known relative ERCC1 expression levels as described in the method above or may now be calibrated against a previously calibrated calibrator RNA such as Human Liver Total RNA (Stratagene, Cat. #735017) described above.

For example, if a subsequent K_{ERCC1} is determined for a different internal control gene and/or a different calibrator RNA, one must calibrate both the internal control gene and the calibrator RNA to tissue samples for which ERCC1 expression levels relative to that particular internal control gene have already been determined. Such a determination can be made using standard pre-TaqMan®, quantitative RT-PCR techniques well known in the art. The known expression levels for these samples will be divided by their corresponding UGE levels to determine a K for that sample. K values are then averaged depending on the number of known samples to determine a new K_{ERCC1} specific to the different internal control gene and/or calibrator RNA.

### Example 7

All patients were enrolled in the Cisplatin/Gemcitabine arm of a prospective multicenter three arm randomized trial (GEPC/98-02, Spanish Lung Cancer Group Phase III trial of Cisplatin/Gemcitabine (CG) versus Cisplatin/Gemcitabine/Vinorelbine (CGV) versus sequential doublets of Gemcitabine/Vinorelbine followed by Ifosfamide/Vinorelbine (GV/IV) in advanced NSCLC). All patients received Gem 1250 mg/m² days 1,8 plus CDDP 100 mg/m² day 1 every 3 weeks. Eligibility criteria for GEPC/98-02 were measurable stage 1V (with brain metastases eligible if asymptomatic) or stage IIIB (malignant pleural and/or pericardial effusion and/or supraclavicular adenopathy) NSCLC and Eastern Cooperative Group (ECOG) performance score 0 2. All patients had chest x-ray and a computed tomography (CT) scan of the chest and upper abdomen before entry into the study and underwent repeat evaluations at least every 6 weeks. Tumor response was assessed according to WHO criteria as complete response, partial response, stable disease, and progressive disease. Tumors were reassessed during treatment with the same imaging methods used to establish the baseline tumor measurement.

Total mRNA was isolated from microdissected FPE pretreatment tumor samples, and Corrected Relative ERCC1 Expression was measured using quantitative RT-PCR. One method for mRNA isolation from such samples is described herein and in U.S. patent application Ser. No. 09/469,338, filed Dec. 20, 1999, and is hereby incorporated by reference.

### Statistical Analysis

The Mann-Whitney U test was used to test for significant associations between the continuous test variable Corrected Relative ERCC1 Expression and dichotomous variables (patient sex, age above and below the median age, presence of weight loss, presence of pleural effusion, tumor stage). The Kruskal-Wallis test was used to test for significant differences in Corrected Relative ERCC1 Expression within multiple groups (ECOG performance status, histopathology). Fisher's exact test was used for the analysis of categorical clinicopathological values including response and dichotomized Corrected Relative ERCC1 Expression values.

All patients were followed from first study treatment until death or until the data were censored. Kaplan-Meier survival curves and the log rank test were used to analyze univariate distributions for survival and disease-free survival. The maximal chi-square method of Miller and Siegmund (Biometrics 1982; 38:1011-1016 and Halpern (Biometrics 1982; 38:1017-1023) was adapted to determine which expression value best segregated patients into poor- and good prognosis subgroups (in terms of likelihood of surviving), with the log-rank test as the statistic used to measure the strength of the grouping. To determine a P value that would be interpreted as a measure of the strength of the association based on the maximal chi-square analysis, 1000 boot-strap-like simulations were used to estimate the distribution of the maximal chi-square statistics under the hypothesis of no association. (Biometrics 1982; 38:1017-1023) Cox's proportional hazards modeling of factors that were significant in univariate analysis was performed to identify which factors might have a significant influence on survival. SPSS version 10.0.5 software (SPSS Inc., Chicago Ill.) was used for all statistical analyses. All P values were two-sided.

### Corrected Relative ERCC1 Expression Levels.

ERCC1 mRNA expression was detectable in all 56 samples analyzed. The median Corrected Relative ERCC1 Expression, relative to the expression of the internal control housekeeping gene β-actin, was 6.7×10⁻³ (range 0.8×10⁻³ to 24.6×10⁻³). There were no significant associations between Corrected Relative ERCC1 Expression levels and any of the factors age (P=0.66), sex (P=0.18) presence of weight loss in the six months prior to randomization (P=0.74), tumor stage (IIIB versus IV, P=0.39), or presence of pleural effusion (P=0.25, all Mann-Whitney U test). There were also no significant differences between the Corrected Relative ERCC1 Expression levels among patients with different performance status grades (P=0.48, Kruskal-Wallis test) or different tumor cell types (all four tumor types, P=0.10, Kruskal-Wallis test), but Corrected Relative ERCC1 Expression levels were significantly higher in SCC tumors (median 8.6×10⁻³) compared to adenocarcinomas (median 5.2×10⁻³, P=0.015, Mann-Whitney test).

### Response to Chemotherapy

The overall response rate for the 47 patients who were evaluable was 44.7%. The Corrected Relative ERCC1 Expression levels in the complete response and partial response i.e. “responding” tumors (median 4.3×10⁻³, range 1.2×10⁻³ to 24.6×10⁻³) were not significantly different from the levels in the stable disease and progressive disease i.e. “non-responding” tumors (median 7.85×10⁻³, range 0.8×10⁻³ to 24.3×10⁻³, P=0.31 Mann-Whitney test). There were also no significant differences between the proportion of responding and non-responding tumors with Corrected Relative ERCC1 Expression values greater and less than any ERCC1 level (all Fisher's exact test). The response rate in tumors with Corrected Relative ERCC1 Expression below the threshold value (“low” expression, 52% responders) was higher than for tumors with Corrected Relative ERCC1 Expression above the threshold value (“high” expression, 36.4% responders, Fisher's exact test, P=0.38).

### Association Between Patient Overall Survival and Corrected Relative ERCC1 Expression Levels

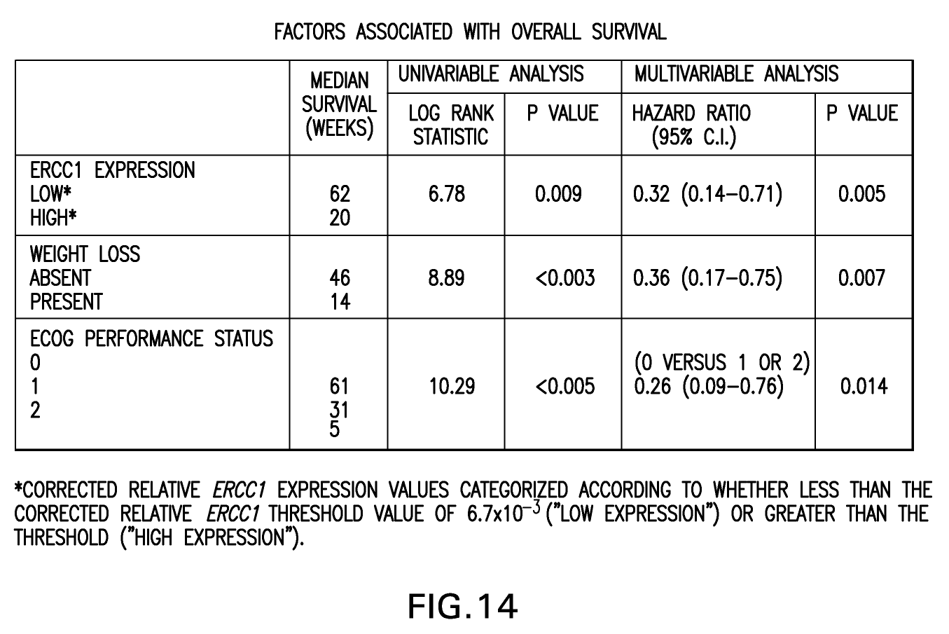

The median overall survival time was 36.6 weeks (range 0-113.4 weeks) and the median time to progression was 24.4 weeks (range 0-102.9 weeks). Use of the log rank test and the maximal chi-square statistic to identify threshold Corrected Relative ERCC1 Expression levels that segregated patients into poor- and good-prognosis subgroups showed that the range of discriminatory values included the median value, which was therefore used as the threshold value for the survival analysis. Therefore, the threshold Corrected Relative ERCC1 Expression value was determined to be 6.7×10⁻³ for NSCLC. FIG. 1 shows the Kaplan-Meier survival curve for patients with intratumoral Corrected Relative ERCC1 Expression levels above and below the threshold Corrected Relative ERCC1 Expression level. As shown in FIG. 14, patients with Corrected Relative ERCC1 Expression levels below the threshold value had a significantly longer median survival of 61.6 weeks (95% C.I. 42.4, 80.7 weeks) compared to 20.4 weeks (95% C.I. 6.9, 33.9 weeks) for patients with Corrected Relative ERCC1 Expression levels above the threshold value. Adjusted for tumor stage, the log rank statistic for the association between low or high Corrected Relative ERCC1 Expression and overall survival was 3.97 and the P value was 0.046. The unadjusted log rank results are shown in FIG. 14.

A separate Corrected Relative ERCC1 Expression threshold value of 5.8×10⁻³ was tested because this value was shown in a previous study to be associated with overall survival for patients with gastric cancer. (Metzger et al., J Clin Oncol 1998; 16:309 316). Overall survival was significantly better for the group of NSCLC patients in this study with Corrected Relative ERCC1 Expression levels less than 5.8×10⁻³ compared to those with ERCC1 levels less than 5.8×10⁻³ (log rank statistic 6.37, P=0.011), although a higher 6.7×10⁻³ Corrected Relative ERCC1 Expression threshold level is a more powerful discriminator.

Other factors that were significantly associated with overall survival on univariable analysis using Kaplan Meier survival curves and the log rank test were the presence of pretreatment weight loss and the ECOG performance status. Patient age (P=0.18), sex (P=0.87), tumor stage (P=0.99), tumor cell type (P=0.63), and presence of pleural effusion (P=0.71) were not significant prognostic factors for overall survival. Corrected Relative ERCC1 Expression level, ECOG performance status, and weight loss remained significant prognostic factors for survival in the Cox proportional hazards regression model multivariable analysis (FIG. 14). P values for a Cox regression model stratified on tumor stage were 0.038 for ERCC1, 0.017 for weight loss, and 0.02 for ECOG performance status (PS 0 versus 1 or 2).

This study found an association between lower ERCC1 mRNA expression levels and improved survival after treatment with a platinum-based chemotherapeutic for patients with cancer.

### Example 8

### Determining the Uncorrected Gene Expression (UGE) for DPD

Two pairs of parallel reactions are carried out. The “test” reactions and the “calibration” reactions. The DPD amplification reaction and the β-actin internal control amplification reaction are the test reactions. Separate β-actin and DPD amplification reactions are performed on the calibrator RNA and are referred to as the calibration reactions. The Taqman instrument will yield four different cycle threshold (Ct) values: Ct_{DPD} and Ct_{β-actin} from the test reactions and Ct_{DPD} and Ct_{β-actin} from the calibration reactions.

The differences in Ct values for the two reactions are determined according to the following equation:

ΔCtₜₑₛₜ=Ct_{DPD}−Ct_{β-actin} (From the “test” reaction)

ΔCt_{calibrator}=Ct_{DPD}−Ct_{β-actin} (From the “calibration” reaction)

Next the step involves raising the number 2 to the negative ΔCt, according to the following equations.

2^{-ΔCt}ₜₑₛₜ (From the “test” reaction)

2^{-ΔCt}_{calibrator} (From the “calibration” reaction)

In order to then obtain an uncorrected gene expression for DPD from the Taqman instrument the following calculation is carried out:

Uncorrected gene expression (UGE) for DPD=2^{-ΔCt}ₜₑₛₜ/2^{-ΔCt}_{calibrator}

Normalizing UGE with Previously Published Values

The normalization calculation entails a multiplication of the UGE with a correction factor (K_{DPD}) specific to DPD and a particular calibrator RNA. The correction factor K_{DPD} can be determined using any internal control gene and any accurately pre-quantified calibrator RNA. Preferably, the internal control gene β-actin and the accurately pre-quantified calibrator RNA, Universal PE RNA; Cat #4307281, lot #3617812014 from Applied Biosystems, are used.

Normalization is accomplished using modification of the ΔCt method described by Applied Biosystems, the Taqman manufacturer, in User Bulletin #2 and described above. To carry out this procedure, the UGE of 6 different previously published test tissues was analyzed for DPD expression using the Taqman methodology described above. The internal control gene β-actin and the calibrator RNA, Universal PE RNA; Cat #4307281, lot #3617812014 from Applied Biosystems was used.

The relative DPD expression level (PV) of each sample previously described in Salonga et al., which is hereby incorporated by reference in its entirety, L7, L91, L121, L150, L220 and L164, was divided by its corresponding Taqman derived UGE to yield an unaveraged correction factor K.

K_{unaveraged}=PV/UGE

Next, all of the K values are averaged to determine a single K_{DPD} correction factor specific for DPD, Universal PE RNA; Cat #4307281, lot #3617812014 calibrator RNA and β-actin.

Therefore, to determine the Corrected Relative DPD Expression in an unknown tissue sample on a scale that is consistent with previously published pre-Taqman DPD expression studies, one merely multiplies the uncorrected gene expression data (UGE) derived from the Taqman apparatus with the K_{DPD} specific correction factor, given the use of the same internal control gene and calibrator RNA.

Corrected Relative DPD Expression=UGE×K_{DPD}

A K_{DPD} may be determined using any accurately pre-quantified calibrator RNA. Future sources of accurately pre-quantified RNA can be calibrated to published samples as described in the method above or may now be calibrated against a previously calibrated calibrator RNA such as Universal PE RNA; Cat #4307281, lot #3617812014 described above.

### Example 9

### DPD Expression in FPE Colorectal Tumor Samples

The methods described above used to analyze 34 tumor samples from 34 patients with advanced colorectal cancer. All patients were treated with an intravenous 5-FU/LV combination regimen as part of a prospective multicenter European 5-FU/CPT11 crossover trial V239. All patients were treated with intravenous 5-FU 425 mg/m² given over a 15 minute infusion for 5 consecutive days with Leucovorin 20 mg/m², also given by infusion over 5 consecutive days. This regimen was given either as first or second line palliative therapy.

Nine (25.5%) of the patients responded to 5-FU/LV, with response defined as any response, including complete response, partial response, and minimal response. Patients with progressive disease or stable disease were classified as non-responders (25 patients, 73.5%). Total mRNA was isolated from microdissected FPE pretreatment tumor samples, and relative mRNA expression levels of DPD/β-actin were measured using quantitative PCR, as described

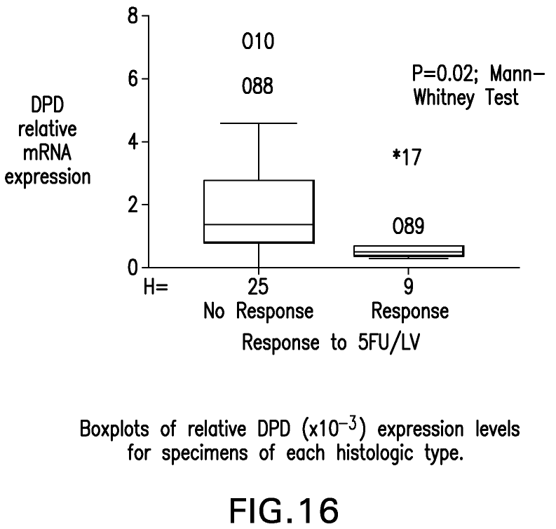

The mean corrected DPD:β-actin levels for the groups of responding and non-responding patients were 0.87×10⁻³ and 2.04×10⁻³, respectively. The Mann-Whitney U test, which compares the rank of values within two independent sample sets, was used to compare the corrected relative DPD expression levels in the responding and non-responding patient groups. Relative DPD levels were significantly lower in the group of responders compared to the non-responders (P=0.02). The association between DPD mRNA expression and response to 5-FU/LV in these patients is shown in the FIG. 16. These data show that DPD expression is a prognostic factor for response to 5-FU-based chemotherapy.

### Example 10

### Determining the Uncorrected Gene Expression (UGE) for TS

Two pairs of parallel reactions are carried out. The “test” reactions and the “calibration” reactions. FIG. 24. The TS amplification reaction and the β-actin internal control amplification reaction are the test reactions. Separate TS and β-actin amplification reactions are performed on the calibrator RNA template and are referred to as the calibration reactions. The TaqMan®. instrument will yield four different cycle threshold (Ct) values: Ct_{TS} and Ct_{β-actin} from the test reactions and Ct_{TS} and Ct_{β-actin} from the calibration reactions. The differences in Ct values for the two reactions are determined according to the following equation:

ΔCtₜₑₛₜ=Ct_{TS}−Ct_{β-actin} (From the “test” reaction)

ΔCt_{calibrator}=Ct_{TS}−Ct_{β-actin} (From the “calibration” reaction)

Next the step involves raising the number 2 to the negative ΔCt, according to the following equations.

2^{-ΔCt}ₜₑₛₜ (From the “test” reaction)

2^{-ΔCt}_{calibrator} (From the “calibration” reaction)

In order to then obtain an uncorrected gene expression for TS from the TaqMan®. instrument the following calculation is carried out:

Uncorrected gene expression (UGE) for TS=2^{-ΔCt}ₜₑₛₜ/2^{-ΔCt}_{calibrator}

Normalizing UGE with Known Relative TS Expression Levels

The normalization calculation entails a multiplication of the UGE with a correction factor (K_{TS}) specific to TS and a particular calibrator RNA. A correction factor K_{TS} can also be determined for any internal control gene and any accurately pre-quantified calibrator RNA. Preferably, the internal control gene β-actin and the accurately pre-quantified calibrator RNA, Universal PE RNA; Cat #4307281, lot #3617812014 from Applied Biosystems are used. Given these reagents correction factor K_{TS} equals 12.6×10⁻³.

Normalization is accomplished using a modification of the ΔCt method described by Applied Biosystems, the TaqMan®. manufacturer, in User Bulletin #2 and described above. To carry out this procedure, the UGE of 6 different previously published test tissues were analyzed for TS expression using the TaqMan®. methodology described above. These tissue samples are described in Salonga, et al., Clinical Cancer Research, 6:1322 1327, 2000, which is hereby incorporated by reference in its entirety. The internal control gene β-actin and the calibrator RNA, Universal PE RNA; Cat #4307281, lot #3617812014 from Applied Biosystems was used.

The previously published relative TS expression level of each sample L7, L91, L121, L150, L220, L164 was divided by its corresponding TaqMan®. derived UGE to yield an unaveraged correction factor K. Salonga, et al, Clinical Cancer Research, 6:1322 1327, 2000, incorporated herein by reference in its entirety.

K_{unaveraged}=Known Values/UGE

Next, all of the K values are averaged to determine a single K_{ERCC1} correction factor specific for TS, Applied Biosystems Universal PE RNA; Cat #4307281, lot #3617812014 calibrator RNA, and β-actin.

Therefore, to determine the Corrected Relative TS Expression in an unknown tissue sample on a scale that is consistent with pre-TaqMan®. TS expression studies, one merely multiplies the uncorrected gene expression data (UGE) derived from the TaqMan®. apparatus with the K_{TS} specific correction factor, given the use of the same internal control gene and calibrator RNA.

Corrected Relative TS Expression=UGE×K_{TS}

A K_{TS} may be determined using any accurately pre-quantified calibrator RNA or internal control gene. Future sources of accurately pre-quantified RNA can be calibrated to samples with known relative ERCC1 expression levels as described in the method above or may now be calibrated against a previously calibrated calibrator RNA such as Universal PE RNA; Cat #4307281, lot #3617812014 from Applied Biosystems described above.

For example, if a subsequent K_{TS} is determined for a different internal control gene and/or a different calibrator RNA, one must calibrate both the internal control gene and the calibrator RNA to tissue samples for which TS expression levels relative to that particular internal control gene have already been determined or published. Such a determination can be made using standard pre-TaqMan®, quantitative RT-PCR techniques well known in the art. The known expression levels for these samples will be divided by their corresponding UGE levels to determine a K for that sample. K values are then averaged depending on the number of known samples to determine a new K_{TS} specific to the different internal control gene and/or calibrator RNA.

### Example 11

### Patient Selection and Chemotherapy Treatment

All patients were enrolled in the compassionate protocol 3C-98-3 at the University of Southern California Medical Center from 1998-2000 and received the following oxaliplatin/5-FU combination therapy regimen: 130 mg/m² oxaliplatin plus continuous infusion of 5-FU. All patients had failed a prior treatment with 5-FU and 60% (30/50) had failed an additional second line treatment with irinotecan (CPT-11). All patients showed active disease in stage 1V colorectal cancer at time of protocol entry.

Clinical Evaluation and Response Criteria

During chemotherapy, weekly evaluations were recorded for performance status, weight, abdominal pain, complete blood counts, and serum creatinine and blood urea nitrogen levels. Tumor burden is measured using computed tomography (CT). A bi-dimensionally measurable tumor mass was required at the time of protocol entry. Responders to therapy were classified as those patients whose tumor burden was decreased by 50% or more for at least 6 weeks. Non-responders included those with stable disease or cancer progression. Survival was computed as the number of days from the initiation of chemotherapy with 5-FU/oxaliplatin to death of any cause. Patients who were alive at the last follow-up evaluation were censored at that time.

Statistical Analysis

TaqMan® analyses yield levels that are expressed as ratios between two absolute measurements (gene of interest: internal reference gene). The Mann-Whitney test and Kruskal-Wallis test were used to evaluate the associations of TS and ERCC1 expression (as continuous variables) with patients demographics. Zar, Biostatistical Analysis. Prentice-Hall, Inc Englewood Cliffs, N.J. (1974), pp. 109-114 and 139-142, respectively. The maximal chi-square method of Miller and Sigmund (Biometrics 38: 1011-1016, 1982) and Halpern (Biometrics 38: 1017-1023, 1982), was adapted to determine which cut-off threshold level best dichotomized patients into low and high TS and ERCC1 expression subgroups. Pearson's chi-square test was used to assess the associations between the dichotomized molecular markers and to response to chemotherapy Zar, Biostatistical Analysis. Prentice-Hall, Inc Englewood Cliffs, N.J. (1974), pp. 59-68. Hazard ratios were used to calculate the relative risks of death. Schulman, Infection Control & Hospital Epidemiology, 18:65-73, 1997. These calculations were based on the Pike estimate, with the use of the observed and expected number of events as calculated in the log-rank test statistic (Pike, J R Stat Soc Series A 135: 201-203, 1972). To determine a P value that would be interpreted as a measure of the strength of the association based art the maximal chi-square analysis, 1000 boot-strap-like simulations were used to estimate the distribution of the maximal chi-square statistics under the hypothesis of no association. (Halpern, Biometrics 38: 1017 1023, 1982). The level of significance was set to p<0.05.

Demographics and Patients Available for Response and Survival Evaluation

A total of 50 patients, consisting of 14 (28%) women and 36 (72%) men, with a median age of 59 (min.:34; max.:83) years were evaluated in this study. The ethnic backgrounds of this group included 39 Caucasians, 6 Hispanics, 3 Asians, and 2 African-Americans. All 50 patients were assessable to associate TS expression and ERCC1 expression levels with survival. Forty-five (90%) were assessable to test the association of this molecular parameters with response by above cited criteria.

TS Expression Levels and ERCC1 Expression Levels

Total mRNA was isolated from microdissected FPE pretreatment tumor samples, and relative mRNA expression levels of ERCC1:β-actin and or TS:β-actin were measured using quantitative RT-PCR. A method for mRNA isolation from such samples is described herein and in U.S. patent application Ser. No. 09/469,338, filed Dec. 20, 1999, and is hereby incorporated by reference in its entirety. A reverse transcription/polymerase chain reaction (RT/PCR)-based assay system was used to determine the level of expression of ERCC1, and β-actin, as described previously. Corrected relative ERCC1 and/or TS expression was determined as described above.

TS gene expression was detectable in all 50 samples analyzed. The median corrected TS expression, relative to the housekeeping gene, β-Actin, was 3.4×10⁻³ (min.:0.18×10⁻³; max.:11.5×10⁻³). Corrected ERCC1 gene expression was detectable in 47 (94%) samples analyzed. The median corrected ERCC1 gene expression was 2.53×10⁻³ (min.: 0.00; max.:14.61×10⁻³). When analyzed by gender, age, and ethnic origin, no significant differences in corrected TS and ERCC1 mRNA expression were found.

Survival in Relation to TS Expression

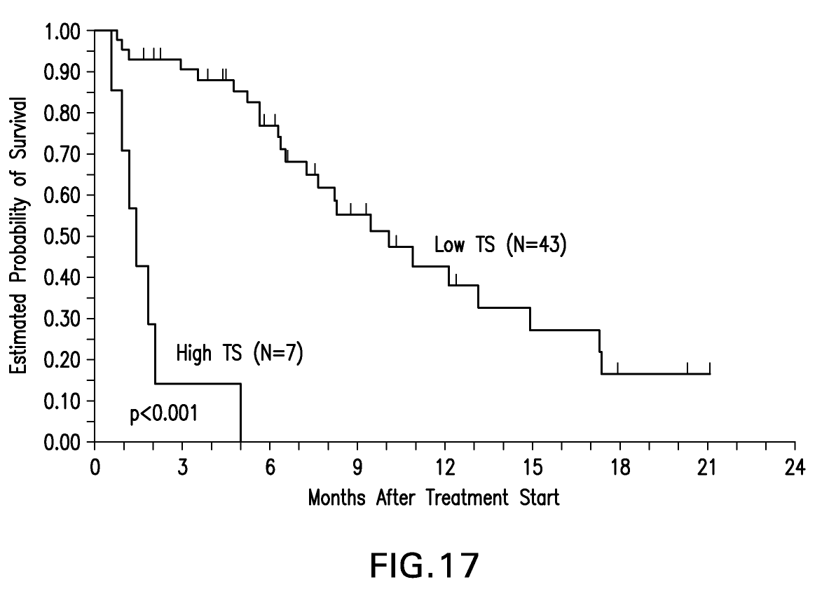

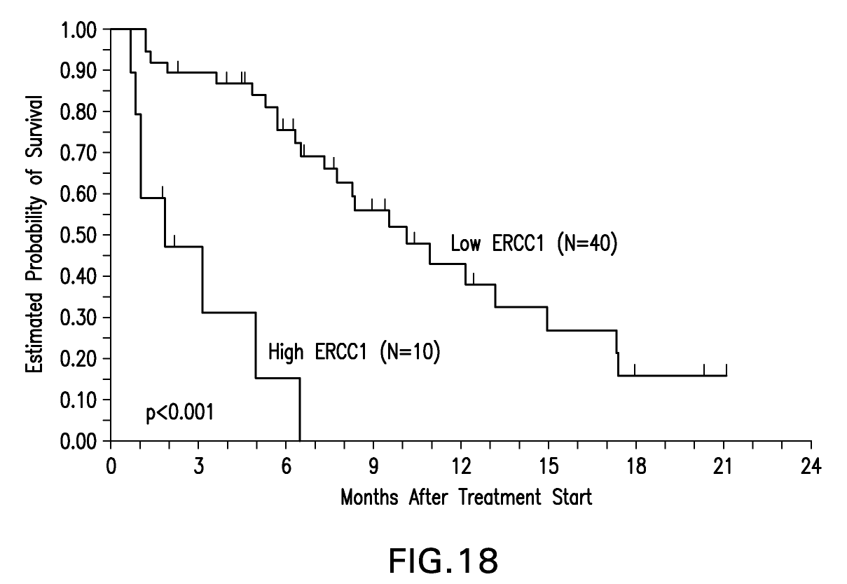

With a median follow-up period of 10.5 months (95% C.I.: 1.8, 21.2) for the 50 patients analyzed in this study, the median survival was 8.4 months (95% C.I.: 6.4, 12.3). Using a TS threshold value of 7.5×10⁻³, 43 (86%) patients had a low corrected TS expression level, and 7 (14%) patients had a high corrected TS expression level. The log-rank test was used to evaluate the association between corrected TS gene expression and survival. The respective survival curves are presented in FIG. 17 and show a median survival of 10.2 months (95% C.I.: 7.4, 15.1) in the low corrected TS expresser group, and 1.5 months (95% C.I.: 1.1,2.1) in the high corrected TS expression group (P<0.001; Logrank Test). The probability of survival at 6 months was 0.77 for patients with corrected TS expression.ltoreq.7.5×10⁻³ compared to 0.00 for the high expresser group. Patients with corrected TS levels>7.5×10⁻³ had a 8.4 (95% CI:2.63,27.13) fold increased relative risk of dying compared to patients with TS levels.ltoreq.7.5×10⁻³ in the univariate analysis (p<0.001, FIG. 17).

Survival in Relation to ERCC1 Expression

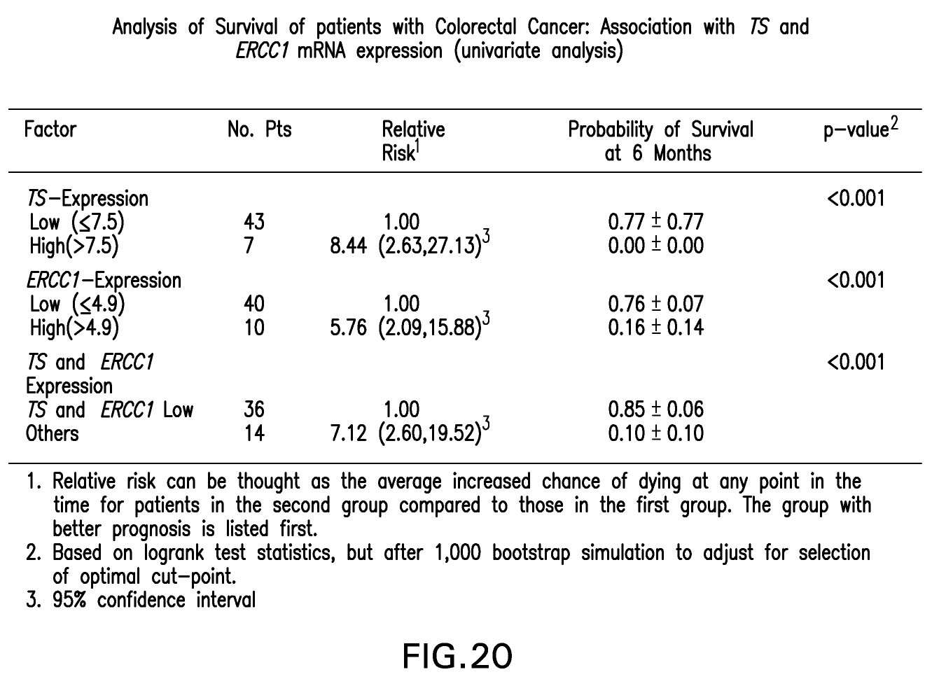

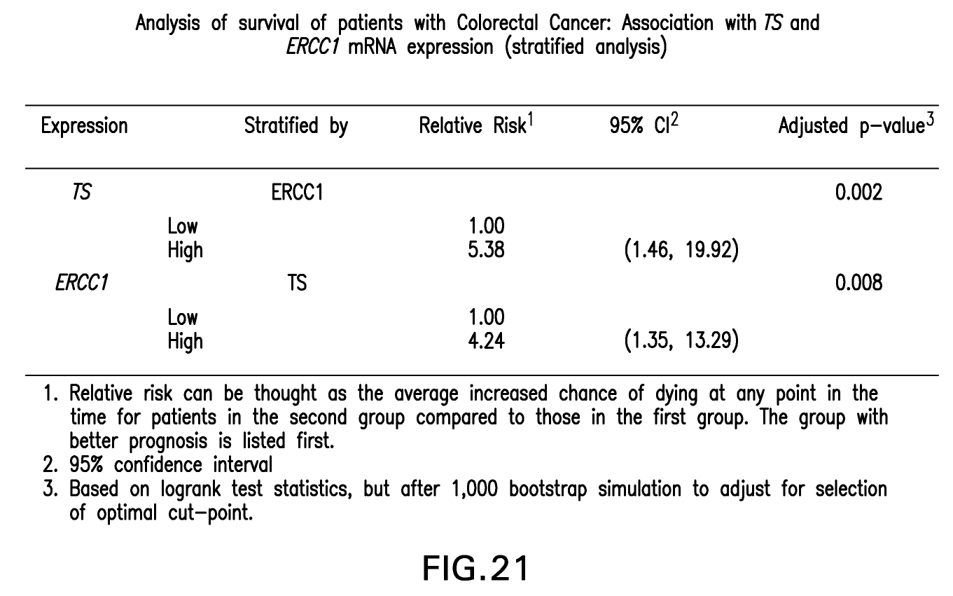

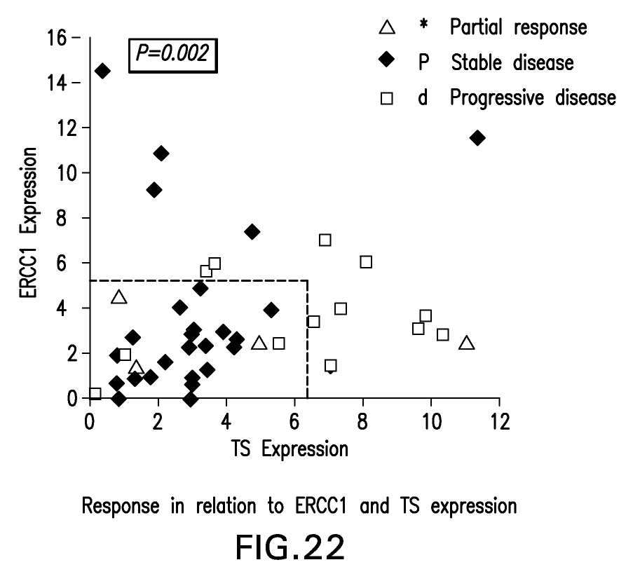

Using 4.9×10⁻³ as a threshold, 40 (80%) had a low corrected ERCC1 expression and 10 (20%) had a high corrected ERCC1 expression. FIG. 22 displays a Kaplan Meier plot of the estimated probability of survival versus corrected ERCC1 expression levels, and shows a median survival of 10.2 months (95% C.I.:7.8,15.1) for the low expresser group and 1.9 months (95% C.I.:1.1,4.9) for the high expressor group (P<0.001; Logrank Test). The probability of survival at 6 months was 0.76 for patients with corrected ERCC1 expression.ltoreq.4.9×10⁻³ compared to 0.16 for patients with corrected ERCC1 expression>4.9×10⁻³. Patients with corrected ERCC1 levels>4.9×10⁻³ had a 4.8 (95% CI:2.09,15.88) fold increased relative risk of dying compared to patients with corrected ERCC1 levels>4.9×10⁻³ in the univariate analysis (p<0.001; FIG. 20).

Survival in Relation to Combined ERCC1 and TS Expression

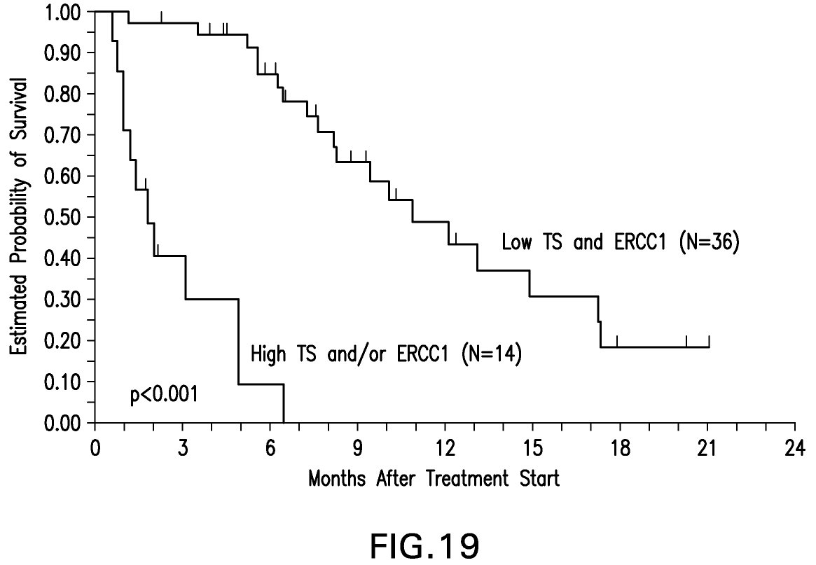

Low corrected TS and ERCC1 expression levels were detected in 36 (72%) of the patients, and 14 (28%) patients had high corrected TS and/or ERCC1 expression level. Patients with low expression levels for both genes had a significant superior survival. The median survival was 11.1 months (95% C.I.:8.4,17.5) for the low corrected TS and ERCC1 expressors, and 1.9 months (95% C.I.:1.1,4.9) for the high corrected TS and/or ERCC1 expressors (P<0.001, Logrank Test; FIG. 19). Patients with low corrected expression levels for both genes had a probability of survival at 6 months of 0.85 compared to 0.10 for the patients with a high corrected expression level for at least one gene, TS or ERCC1. The relative risk of dying for patients with an increased corrected expression for at least one gene (TS or ERCC1) was 7.12 (95% CI:2.60,19.52) compared to patients, which showed low expression levels for both genes in the tumor (P<0.00 1; FIG. 20). TS and ERCC1 mRNA expression are independent of each other as revealed by the stratified analysis (FIG. 24).

Association of Response with TS and ERCC1 Gene Expression Levels.

The median corrected TS expression level was 3.4×10⁻³ (min.: 0.18×10⁻³; max.:11.50×10⁻³) for the 45 measurable patients and is identical to the entire 50 patient-cohort. When responses were analyzed by segregating tumors into low- and high TS expressors, three out of four (75%) partial responders, 26 of 27 (96%) of patients with stable disease, and 9 of 14 (64%) of patients with progressive disease had a low corrected TS expression (P=0.02; Fisher's Exact Test)

The median corrected ERCC1 expression level was 2.7×10⁻³ (min.:0.00; max.:14.61×10⁻³) for the 45 measurable patients and not significantly different to the entire 50 patient-cohort. However the ERCC1 expression level was not statistically significant associated with response to chemotherapy (p=0.29, Fisher's Exact Test).

### Example 12

### Determining the Uncorrected Gene Expression (UGE) for EGFR

Two pairs of parallel reactions are carried out. The “test” reactions and the “calibration” reactions. FIG. 29. The EGFR amplification reaction and the β-actin internal control amplification reaction are the test reactions. Separate EGFR and β-actin amplification reactions are performed on the calibrator RNA template and are referred to as the calibration reactions. The TaqMan® instrument will yield four different cycle threshold (Ct) values: Ct_{EGFR} and Ct_{β-actin} from the test reactions and Ct_{EGFR} and Ct_{β-actin} from the calibration reactions. The differences in Ct values for the two reactions are determined according to the following equation:

ΔCtₜₑₛₜ=Ct_{EGFR}−Ct_{β-actin} (From the “test” reaction)

ΔCt_{calibrator}=Ct_{EGFR}−Ct_{β-actin} (From the “calibration” reaction)

Next the step involves raising the number 2 to the negative ΔCt, according to the following equations.

2^{-ΔCt}ₜₑₛₜ (From the “test” reaction)

2^{-ΔCt}_{calibrator} (From the “calibration” reaction)

In order to then obtain an uncorrected gene expression for EGFR from the TaqMan® instrument the following calculation is carried out:

Uncorrected gene expression (UGE) for EGFR=2^{-ΔCt}ₜₑₛₜ/2^{-ΔCt}_{calibrator}

Normalizing UGE with Known Relative EGFR Expression Levels

The normalization calculation entails a multiplication of the UGE with a correction factor (K_{EGFR}) specific to EGFR and a particular calibrator RNA. A correction factor K_{EGFR} can also be determined for any internal control gene and any accurately pre-quantified calibrator RNA. Preferably, the internal control gene β-actin and the accurately pre-quantified calibrator RNA, Human Liver Total RNA (Stratagene, Cat #735017), are used. Given these reagents correction factor K_{EGFR} equals 1.54.

Normalization is accomplished using a modification of the ΔCt method described by Applied Biosystems, the TaqMan® manufacturer, in User Bulletin #2 and described above. To carry out this procedure, the UGE of 6 different FPE test tissues were analyzed for EGFR expression using the TaqMan® methodology described above. The internal control gene β-actin and the calibrator RNA, Human Liver Total RNA (Stratagene, Cat #735017) was used.

The already known relative EGFR expression level of each sample AG22 1, AG222, AG252, Adult Lung, PC3, AdCol was divided by its corresponding TaqMan® derived UGE to yield an unaveraged correction factor K.

K_{unaveraged}=Known value/UGE

Next, all of the K values are averaged to determine a single K_{EGFR} correction factor specific for EGFR, Stratgene Human Liver Total RNA (Stratagene, Cat #735017) from calibrator RNA and β-actin.

Therefore, to determine the Corrected Relative EGFR Expression in an unknown tissue sample on a scale that is consistent with pre-TaqMan® EGFR expression studies, one merely multiplies the uncorrected gene expression data (UGE) derived from the TaqMan® apparatus with the K_{EGFR} specific correction factor, given the use of the same internal control gene and calibrator RNA.

Corrected Relative EGFR Expression=UGE×K_{EGFR}

A K_{EGFR} may be determined using any accurately pre-quantified calibrator RNA or internal control gene. Future sources of accurately pre-quantified RNA can be calibrated to samples with known relative EGFR expression levels as described in the method above or may now be calibrated against a previously calibrated calibrator RNA such as Human Liver Total RNA (Stratagene, Cat #735017) described above.

For example, if a subsequent K_{EGFR} is determined for a different internal control gene and/or a different calibrator RNA, one must calibrate both the internal control gene and the calibrator RNA to tissue samples for which EGFR expression levels relative to that particular internal control gene have already been determined. Such a determination can be made using standard pre-TaqMan®, quantitative RT-PCR techniques well known in the art. The known expression levels for these samples will be divided by their corresponding UGE levels to determine a K for that sample. K values are then averaged depending on the number of known samples to determine a new K_{EGFR} specific to the different internal control gene and/or calibrator RNA.

### Example 13

### Determining the Uncorrected Gene Expression (UGE) for HER2-neu

Two pairs of parallel reactions are carried out. The “test” reactions and the “calibration” reactions. FIG. 26. The HER2-neu amplification reaction and the β-actin internal control amplification reaction are the test reactions. Separate HER2-neu and β-actin amplification reactions are performed on the calibrator RNA template and are referred to as the calibration reactions. The TaqMan® instrument will yield four different cycle threshold (Ct) values: Ct_{HER2-neu} and Ct_{β-actin} from the test reactions and Ct_{Her2-neu} and Ct_{β-actin} from the calibration reactions. The differences in Ct values for the two reactions are determined according to the following equation:

ΔCtₜₑₛₜ=Ct_{Her2-neu}−Ct_{β-actin} (From the “test” reaction)

ΔCt_{calibrator}=Ct_{Her2-neu}−Ct_{β-actin} (From the “calibration” reaction)

Next the step involves raising the number 2 to the negative ΔCt, according to the following equations.

ΔCtₜₑₛₜ=Ct_{Her-neu}−CT_{β-actin} (From the “test” reaction)

ΔCt_{calibrator}=Ct_{Her-neu}−CT_{β-actin} (Form the “calibration” reaction)

Next the step involves raising the number 2 to the negative ΔCt, according to the following equations.

2^{-ΔCt}ₜₑₛₜ (From the “test” reaction)

2^{-ΔCt}_{calibrator} (From the “calibration” reaction)

In order to then obtain an uncorrected gene expression for HER2-neu from the TaqMan® instrument the following calculation is carried out:

Uncorrected gene expression (UGE) for Her2-neu=2^{-ΔCt}ₜₑₛₜ/2^{-ΔCt}_{calibrator}

Normalizing UGE with Known Relative Her2-Neu Expression Levels

The normalization calculation entails a multiplication of the UGE with a correction factor (K_{HER2-neu}) specific to HER2-neu and a particular calibrator RNA. A correction factor K_{HER2-neu} can also be determined for any internal control gene and any accurately pre-quantified calibrator RNA. Preferably, the internal control gene β-actin and the accurately pre-quantified calibrator RNA, Human Liver Total RNA (Stratagene, Cat #735017) are used. Using β-actin and the accurately pre-quantified calibrator RNA, Human Liver Total RNA (Stratagene, Cat #735017) the correction factor K_{HER2-neu} equals 12.6×10⁻³.

Normalization is accomplished using a modification of the ΔCt method described by Applied Biosystems, the TaqMan® manufacturer, in User Bulletin #2 and described above. To carry out this procedure, the UGE of 6 different FPE test tissues were analyzed for HER2-neu expression using the TaqMan® methodology described above. The internal control gene β-actin and the calibrator RNA, Human Liver Total RNA (Stratagene, Cat #735017) was used.

The already known relative HER2-neu expression level of each sample AG221, AG222, AG252, Adult Lung, PC3, AdCol is divided by its corresponding TaqMan® derived UGE to yield an unaveraged correction factor K.

K_{unaveraged}=Known value/UGE

Next, all of the K values are averaged to determine a single K_{EGFR} correction factor specific for HER2-neu, Human Liver Total RNA (Stratagene, Cat #735017) calibrator, and β-actin.

Therefore, to determine the Corrected Relative HER2-neu Expression in an unknown tissue sample on a scale that is consistent with pre-TaqMan® HER2-neu expression studies, one merely multiplies the uncorrected gene expression data (UGE) derived from the TaqMan® apparatus with the K_{HER2-neu} specific correction factor, given the use of the same internal control gene and calibrator RNA.

Corrected Relative EGFR Expression=UGE×K_{EGFR}

A K_{HER2-neu} may be determined using any accurately pre-quantified calibrator RNA or internal control gene. Future sources of accurately pre-quantified RNA can be calibrated to samples with known relative EGFR expression levels as described in the method above or may now be calibrated against a previously calibrated calibrator RNA such as Human Liver Total RNA (Stratagene, Cat #735017) described above.

For example, if a subsequent K_{HER2-neu} is determined for a different internal control gene and/or a different calibrator RNA, one should calibrate both the internal control gene and the calibrator RNA to tissue samples for which HER2-neu expression levels relative to that particular internal control gene have already been determined or published. Such a determination can be made using standard pre-TaqMan®, quantitative RT-PCR techniques well known in the art. The known expression levels for these samples will be divided by their corresponding UGE levels to determine a K for that sample. K values are then averaged depending on the number of known samples to determine a new K_{HER2-neu} specific to the different internal control gene and/or calibrator RNA.

### Example 14

### Testing Different Concentrations of EDTA and Different Incubation Temperatures

The procedure described in Example 1 was carried out using four different concentrations of EDTA within the extraction solution (0.1 mM, 0.6 mM, 3.6 mM and 20 mM) and 4 different incubation temperatures (44, 50, 56 and 62° C.). These variables were assessed with two different FFPE samples. Four different primer sets were used—100,300,400 and 1000 bp primers (meaning that the primers were 100, 300, 400 or 1,000 bp away from the 3′ (poly A) end of the RNA). Oligo dT reverse transcripase was performed. The extraction process used Tris/EDTA/PK buffer (described above) 16 hrs at various temperatures. A single Phenol/Chloroform/Isoamyl alcohol (PCI) extraction was performed to remove DNA contamination. The isolated RNA was resuspended in 50 μl Tris.

The data showed that although all incubation temperatures worked and different concentrations of EDTA worked, a preferred parameter to obtain long fragment RNA used 3.6 mm EDTA and a temperature range from 50-56° C. (as seen by the lowest Cts). See Table 2 below.

TABLE 2
EDTA Concentrations/Temperature

| | | | |
|---|---|---|---|
| 44° C. | 50° C. | 56° C. | 62° C. |

M1

| | | | | | |
|---|---|---|---|---|---|
| | 100 bp | | | | |
| EDTA | 0.1 mM | 23.1101 | 22.196 | 22.3712 | 22.9834 |
| | 0.6 mM | 23.3616 | 22.3673 | 22.5973 | 22.6378 |
| | 3.6 mM | 22.7179 | 22.2331 | 21.8783 | 21.9587 |
| | 20 mM | 23.0952 | 22.1579 | 21.8366 | 21.9461 |
| | 300 bp | | | | |
| | 0.1 mM | 26.4604 | 24.9523 | 25.0977 | 25.2382 |
| | 0.6 mM | 26.5112 | 24.8636 | 25.1074 | 24.9243 |
| | 3.6 mM | 25.8619 | 24.9501 | 24.5143 | 24.6931 |
| | 20 mM | 26.7892 | 25.3186 | 25.0386 | 25.3433 |
| | 400 bp | | | | |
| | 0.1 mM | 28.0654 | 27.0076 | 27.3271 | 27.6107 |
| | 0.6 mM | 28.1851 | 26.8109 | 27.0736 | 26.9763 |
| | 3.6 mM | 27.3316 | 26.4201 | 26.2641 | 26.8206 |
| | 20 mM | | | | |
| | 1000 bp | | | | |
| | 0.1 mM | 29.9852 | 27.9539 | 27.8691 | 27.3389 |
| | 0.6 mM | 30.3583 | 27.8463 | 28.1046 | 27.3479 |
| | 3.6 mM | 29.6949 | 28.4397 | 27.9215 | 27.5779 |
| | 20 mM | 30.2612 | 28.8745 | 28.6416 | 28.5171 |

M2

| | | | | | |
|---|---|---|---|---|---|
| | 100 bp | | | | |
| EDTA | 0.1 mM | 21.4018 | 21.4633 | 21.4504 | 21.6834 |
| | 0.6 mM | 21.4292 | 21.0563 | 21.1044 | 21.47 |
| | 3.6 mM | 21.6286 | 20.9075 | 20.9972 | 20.7812 |
| | 20 mM | 21.027 | 21.0579 | 21.0027 | 21.2553 |
| | 300 bp | | | | |
| | 0.1 mM | 23.9609 | 23.8571 | 23.9106 | 24.3064 |
| | 0.6 mM | 23.9403 | 23.4637 | 23.7906 | 24.2977 |
| | 3.6 mM | 24.5158 | 23.5704 | 23.7666 | 23.7389 |
| | 20 mM | 24.3286 | 23.9254 | 24.2174 | 24.5737 |
| | 400 bp | | | | |
| | 0.1 mM | 26.5268 | 25.8475 | 25.9314 | 26.5978 |
| | 0.6 mM | 25.854 | 25.1882 | 25.6386 | 26.0809 |
| | 3.6 mM | 26.1936 | 24.9937 | 25.1652 | 25.3845 |
| | 20 mM | 25.7346 | 25.183 | | |
| | 1000 bp | | | | |
| | 0.1 mM | 30.0974 | 27.9908 | 27.5454 | 27.673 |
| | 0.6 mM | 29.7021 | 27.7281 | 27.5899 | 27.2548 |
| | 3.6 mM | 28.7475 | 27.6364 | 27.2434 | 26.9735 |
| | 20 mM | 28.582 | 27.7993 | 28.078 | 27.9715 |

### Example 15

### Use of Sodium Citrate or EGTA Instead of EDTA as the Chelator

In this experiment, three different chelators were tested: EDTA, EGTA and sodium citrate. EGTA and sodium citrate were tested at 0.1, 0.6, 3.6 and 20 mM along with 3.6 mM EDTA. The samples were incubated for 16 hours at 50° C. A single phenol/chloroform step was used to remove contaminating DNA. The isolated RNA was resuspended in 50 μl Tris. The results showed that sodium citrate at 0.6 and 3.6 mM is a good chelator and it even worked at concentrations as high as 20 mM. See Table 3 below.

TABLE 3

| | | | |
|---|---|---|---|
| 0.1 | 0.6 | 3.6 | 20 |

100 bp

| | | | | |
|---|---|---|---|---|
| M1 | | | | |
| EGTA | 22.05584 | 21.91483 | 22.14477 | 21.32523 |
| NaCitrate | 21.26925 | 20.75951 | 20.68721 | 20.97618 |
| EDTA | | | 20.97683 | |
| M2 | | | | |
| EGTA | 20.95042 | 21.02856 | 21.50124 | 20.75807 |
| NaCitrate | 20.43178 | 20.03613 | 20.35514 | 20.25602 |
| EDTA | | | 20.01918 | |

300 bp

| | | | | |
|---|---|---|---|---|
| M1 | | | | |
| EGTA | 26.4581 | 26.09962 | 26.25091 | 25.07969 |
| NaCitrate | 24.72144 | 22.69152 | 22.93418 | 23.24895 |
| EDTA | | | 23.95666 | |
| M2 | | | | |
| EGTA | 25.16278 | 25.05163 | 25.61697 | 24.87095 |
| NaCitrate | 24.38187 | 22.76978 | 22.86453 | 22.9773 |
| EDTA | | | 23.09989 | |

400 bp

| | | | | |
|---|---|---|---|---|
| M1 | | | | |
| EGTA | 28.26457 | 27.55415 | 28.0468 | 26.84575 |
| NaCitrate | 26.77297 | 25.08387 | 25.27766 | 25.49137 |
| EDTA | | | 26.43221 | |
| M2 | | | | |
| EGTA | 27.03514 | 26.65384 | 27.32611 | 26.27913 |
| NaCitrate | 25.89327 | 24.67949 | 24.87163 | 25.08208 |
| EDTA | | | 25.00621 | |

1000 bp

| | | | | |
|---|---|---|---|---|
| M1 | | | | |
| EGTA | 29.19289 | 29.04155 | 29.78311 | 28.69869 |
| NaCitrate | 27.47323 | 25.47567 | 25.64558 | 25.9896 |
| EDTA | | | 26.5681 | |
| M2 | | | | |
| EGTA | 28.37434 | 28.50888 | 29.09281 | 27.69427 |
| NaCitrate | 27.28292 | 25.09847 | 25.18052 | 25.41669 |
| EDTA | | | 25.68557 | |

### Example 16

### PK Concentration and Incubation Time

The RNA extraction was carried out as described above except the extraction solution comprised Tris/EDTA/PK buffer with 0.5×, 1×, 2× and 4×PK concentrations. 1× PK concentration=500 μg/ml. Incubation times of 3, 6, 12, 16 and 20 hrs were assessed and a single Phenol/Chloroform/Isoamyl alcohol (PCI) extraction was performed. The RNA was resuspended in 50 μl Tris. Oligo dT reverse transcriptase was performed. The results showed that the preferred incubation time was for 16 hours at 50° C. The different concentrations of PK all worked and it appears that 1× worked at well as the higher concentrations. See Table 4.

TABLE 4

| | | | | |
|---|---|---|---|---|
| 3 hrs | 6 hrs | 12 hrs | 16 hrs | 20 hrs |

Sample M3

| | 100 bp | | | | |
|---|---|---|---|---|---|
| 0.5 | 28.31861 | 26.58241 | 25.46082 | 24.74091 | 24.36967 |
| 1 | 28.73512 | 26.28547 | 24.97916 | 24.44251 | 25.0303 |
| 2 | 28.03614 | 25.97902 | 25.04817 | 24.51852 | 24.82751 |
| 4 | 29.53969 | 26.86962 | 25.82528 | 24.64906 | 26.06563 |
| | 300 bp | | | | |
| 0.5 | 31.91504 | 30.79285 | 29.34402 | 28.86285 | 28.53668 |
| 1 | 32.02502 | 30.62955 | 29.29224 | 28.61764 | 28.96055 |
| 2 | 30.01775 | 30.23354 | 28.37638 | 28.20503 | 28.61338 |
| 4 | 33.19733 | 31.13701 | 29.4218 | 28.05663 | 29.63655 |
| | 400 bp | | | | |
| 0.5 | 32.87424 | 32.41137 | 30.89621 | 30.70137 | 30.3052 |
| 1 | 33.28026 | 32.20423 | 31.00278 | 28.61866 | 30.58809 |
| 2 | 32.45806 | 31.61768 | 30.20251 | 29.87417 | 30.30267 |
| 4 | 34.42826 | 32.35595 | 31.04639 | 29.15671 | 31.08682 |
| | 1000 bp | | | | |
| 0.5 | 33.11692 | 32.76052 | 30.58482 | 31.28069 | 30.60988 |
| 1 | 33.10397 | 32.08092 | 30.74326 | 31.22962 | 30.76994 |
| 2 | 31.63774 | 31.48734 | 30.37366 | 30.01824 | 30.33966 |
| 4 | 34.11372 | 32.5022 | 30.43215 | 29.4997 | 31.24582 |

Sample M4

| | 100 bp | | | | |
|---|---|---|---|---|---|
| 0.5 | 27.50618 | 26.33205 | 24.90162 | 24.11188 | 24.08716 |
| 1 | 27.4682 | 26.733 | 24.70451 | 24.20879 | 24.15328 |
| 2 | 27.23042 | 26.39459 | 24.51051 | 24.34997 | 24.89759 |
| 4 | 27.87524 | 27.06252 | 25.15285 | 24.34653 | 24.96841 |
| | 300 bp | | | | |
| 0.5 | 31.97025 | 30.67854 | 28.66794 | 27.46286 | 27.86116 |
| 1 | 31.51245 | 30.91859 | 28.21581 | 27.52881 | 27.74868 |
| 2 | 31.29552 | 30.24914 | 28.07282 | 27.54162 | 27.36115 |
| 4 | 31.53427 | 30.60247 | 28.67055 | 27.59484 | 27.97908 |
| | 400 bp | | | | |
| 0.5 | 33.43846 | 32.34631 | 30.33808 | 29.74591 | 30.02667 |
| 1 | 33.10722 | 32.62965 | 30.38408 | 29.86639 | 29.84286 |
| 2 | 32.72484 | 32.17758 | 30.1776 | 29.86035 | 29.58862 |
| 4 | 32.81466 | 32.15597 | 30.64189 | 29.74172 | 30.08539 |
| | 1000 bp | | | | |
| 0.5 | 33.69785 | 32.728 | 30.44111 | 30.24496 | 30.44284 |
| 1 | 33.38166 | 32.99974 | 30.4358 | 30.20776 | 30.32836 |
| 2 | 33.33786 | 32.39023 | 30.4802 | 30.16997 | 30.18674 |
| 4 | 33.47508 | 32.55459 | 30.54346 | 30.12733 | 29.94782 |

### Example 17

### Isolation of mRNA and gDNA from FFPE Pancreatic Ductal Adenocarcinoma (PDA) Tissue

Using methods of the present invention, RNA was isolated from FFPE Pancreatic ductal adenocarcinoma (PDA) tissue samples. Global mRNA expression and gDNA copy number data were obtained from a single microdissected sample and were compared to data from tissues processed separately on similar platforms. mRNA and gDNA data was found to be of comparable to superior quality to frozen, non-microdissected tumor tissues, as evidenced by median proportion of overlapping probes (for gDNA) and robust copy number/mRNA expression concordance.

Little is known about the expression and copy number patterns of pancreatic ductal adenocarcinoma (PDA) primary tumors, due in large part to the difficulty obtaining tissue from this retroperitoneal organ, and the poor quality of nucleic acids obtained. In addition, severe desmoplasia leads to stromal contamination (Chu, G. C., et al., Stromal biology of pancreatic cancer. J Cell Biochem, 2007. 101(4): p. 887-907) and the extreme autodigestive properties of the organ often lead to degraded nucleic acid quality.

Microdissection of the tumor tissue was performed using manual or laser dissection techniques. After microdissection, gDNA was isolated by a proprietary extraction procedure at Response Genetics (Los Angeles, Calif.). Total RNA was isolated with the method of the present invention. Two rounds of RNA amplification and cDNA preparation was performed as previously described (Lord, R. V., et al., Telomerase reverse transcriptase expression is increased early in the Barrett's metaplasia, dysplasia, adenocarcinoma sequence. J Gastrointest Surg, 2000. 4(2): p. 135-42). cRNA was synthesized and hybridized to Affymetrix Hu133Plus2 chips. Co-extracted gDNA (70 ng) was subjected to genome wide allele-specific copy number analysis on a molecular inversion probe (MIP) platform as described (Wang, Y., et al., Analysis of molecular inversion probe performance for allele copy number determination. Genome Biol, 2007. 8(11): p. R246).

It was found that genomic DNA and mRNA can be successfully and consistently co-isolated from a single microdissected FFPE sample in PDA and analyzed for expression and allele specific copy number on a genome-wide scale. FFPE MIP data compared favorably to non-microdissected, frozen tumor samples. Gene expression reflects copy number as in samples extracted separately. This approach of microdissection and co-extraction of nucleic acids (mRNA and gDNA) makes archival FFPE tissue available to genome wide analysis on multiple platforms and begins to maximize data from valuable patient samples extant in large pathological archives available for genomic analysis, which is especially relevant for studies in hereditary disease and clinical trials where fresh material is rarely available.

### Example 18

### Sequences of Primers Discussed Above

| | | |
|---|---|---|
| ERCC1-504F | SEQ ID NO:1 | gggaatttgg cgacgtaatt c |
| ERCC1-574R | SEQ ID NO:2 | gcggaggctg aggaacag |
| GST-F | SEQ ID NO:3 | cctgtaccag tccaatacca tcct |
| GST-R | SEQ ID NO:4 | tcctgctggt ccttcccata |
| DPD3A | SEQ ID NO:5 | aggacgcaag gagggtttg |
| DPD3a-13R | SEQ ID NO:6 | gtccgccgag tccttactga |
| DPD3b-651F | SEQ ID NO:7 | gaagcctatt ctgcaaagat tgc |
| DPD3b-736R | SEQ ID NO:8 | gagtacccca atcgagccaa a |
| TS-763F | SEQ ID NO:9 | ggcctcggtg tgccttt |
| TS-825R | SEQ ID NO:10 | gatgtgcgca atcatgtacg t |
| EGFR-1753F | SEQ ID NO:11 | tgcgtctctt gccggaat |
| EGFR-1823R | SEQ ID NO:12 | ggctcaccct ccagaagctt |
| Her2-neu | SEQ ID NO:13 | ctgaactggt gtatgcagat tgc |
| 2671F | | |
| Her2-neu | SEQ ID NO:14 | ttccgagcggccaagtc |
| 2699R | | |

All references cited herein are hereby incorporated by reference in their entirety. Throughout the specification, the references are referred to with a reference number. These references are provided below.

### REFERENCES

1. Ross J S. The impact of molecular diagnostic tests on patient outcomes. Clin Lab Med. 1999; 19:815-831.
2. Lewis F, Maughan N J, Smith V, Hillan K, Quirke P: Unlocking the archive: gene expression in paraffin-embedded tissue. J Pathol 2001, 195:66-71.
3. Srinivasan M, Sedmak D, Jewell S. Effects of fixative and tissue processing on the content and integrity of nucleic acids. Am J Pathol. 2002; 161:1961-1971.
4. Masuda N, Ohnishi T, Kawamoto S, et al. Analysis of chemical modification of RNA from formalin-fixed samples and optimization of molecular biology applications for such samples. Nucleic Acids Res. 1999; 27:4436-4443.
5. Gillespie J W, Best C J, Bichsel V E, et al. Evaluation of non-formalin tissue fixation for molecular profiling studies. Am J Pathol. 2002; 160:449-457.
6. Stanta G, Schneider C: RNA extracted from paraffin-embedded human tissues is amenable to analysis by PCR amplification. Biotechniques 1991, 11:304-308.
7. Rupp G M, Locker J: Purification and analysis of RNA from paraffin-embedded tissues. Biotechniques 1988, 6:56-60.
8. Von Weizsacker F, Labeit S, Koch H K, Oehlert W, Gerok W, Blum H E: A simple and rapid method for the detection of RNA in formalin-fixed, paraffin-embedded tissues by PCR amplification. Biochem Biophys Res Commun 1991, 174:176-180.
9. Ben-Ezra J, Johnson D A, Rossi J, Cook N, Wu A: Effect of fixation on the amplification of nucleic acids from paraffin-embedded material by the polymerase chain reaction. J Histochem Cytochem 1991, 39:351-354.
10. Finke J, Fritzen R, Ternes P, Lange W, Dolken G: An improved strategy and a useful housekeeping gene for RNA analysis from formalin-fixed, paraffin-embedded tissues by PCR. Biotechniques 1993, 14:448-453.
11. Mies C: A simple, rapid method for isolating RNA from paraffin-embedded tissues for reverse transcription-polymerase chain reaction (RT-PCR). J Histochem Cytochem 1994, 42:811-813.
12. Foss R D, Guha-Thakurta N, Conran R M, Gutman P: Effects of fixative and fixation time on the extraction and polymerase chain reaction amplification of RNA from paraffin-embedded tissue: comparison of two housekeeping gene mRNA controls. Diagn Mol Pathol 1994, 3:148-155.
13. Stanta G, Bonin S: RNA quantitative analysis from fixed and paraffin-embedded tissues: membrane hybridization and capillary electrophoresis. Biotechniques 1998, 24:271-276.
14. Godfrey T E, Kim S-H, Chavira M, Ruff D W, Warren R S, Gray J W, Jensen R H: Quantitative mRNA expression analysis from formalin-fixed, paraffin-embedded tissues using 5′ nuclease quantitative reverse transcription-polymerase chain reaction. J Mol Diagn 2000, 2:84-91.
15. Specht K, Richter T, Muller U, Walch A, Werner M, Hofler H: Quantitative gene expression analysis in microdissected archival formalin-fixed and paraffin-embedded tumor tissue. Am J Pathol 2001, 158:419-429.
16. Karbler T, Grskovic M, Dominis M, Antica M: A simple method for RNA isolation from formalin-fixed and paraffin-embedded lymphatic tissues. Exp Mol Pathol 2003, 74:336-340.
17. Gillespie J W, Best C J, Bichsel V E, Cole K A, Greenhut S F, Hewitt S M, Ahram M, Gathright Y B, Merino M J, Strausberg R L, Epstein J I, Hamilton S R, Gannot G, Baibakova G V, Calvert V S, Flaig M J, Chuaqui R F, Herring J C, Pfeifer J, Petricoin E F, Linehan W M, Duray P H, Bova G S, Emmert-Buck M R: Evaluation of non-formalin tissue fixation for molecular profiling studies. Am J Pathol 2002, 160:449-457.
18. De Andres B, del Pozo V, Gallardo S, de Arruda-Chaves E, Cardaba B, Martin-Orozco E, Posada M, Palomino P, Lahoz C: Improved method for mRNA extraction from paraffin-embedded tissues. Biotechniques 1995, 18:42-44.
19. Banerjee S K, Makdisi W F, Weston A P, Mitchell S M, Campbell D R: Microwave-based DNA extraction from paraffin-embedded tissue for PCR amplification. Biotechniques 1995, 18:768-770.
20. Masuda N, Ohnishi T, Kawamoto S, Monden M, Okubo K: Analysis of chemical modification of RNA from formalin-fixed samples and optimization of molecular biology applications for such samples. Nucleic Acids Res 1999, 27:4436-4443.
21. Coombs N J, Gough A C, Primrose J N: Optimization of DNA and RNA extraction from archival formalin-fixed tissue. Nucleic Acids Res 1999, 27:e12.
22. Shi S-R, Cote R J, Wu L, Data R, Shi Y, Liu D, Lim H, Taylor C R: DNA extraction from archival formalin-fixed, paraffin-embedded tissues sections based on the antigen retrieval principle: heating under the influence of pH. J Histochem Cytochem 2000, 50:1005-1011.
23. Wu L, Pattern N, Yamashiro C T, Chui B: Extraction and amplification of DNA from formalin-fixed, paraffin-embedded tissues. Appl Immunohistochem Mol Morphol 2002, 10:269-274.
24. Liu H, Huang X, Zhang Y, Ye H, EL Hamifi A, Kocjan G, Dogan A, Isaacson P G, Du M-Q: Archival fixed histologic and cytologic specimens including stained and unstained materials are amenable to RT-PCR. Diagn Mol Pathol 2002, 11:222-227.
25. Bonin S, Petrera F, Niccolini B, Stanta G: PCR analysis in archival postmortem tissues. Mol Pathol 2003, 56:184-186.
26. Macabeo-Ong M, Ginzinger D G, Dekker N, McMillan A, Regezi J A, Wong D T W, Jordan R C K: Effect of duration of fixation on quantitative reverse transcription polymerase chain reaction analyses. Mod Pathol 2001, 15:979-987.
27. Srinivasan M, Sedmak D, Jewell S: Effect of fixatives and tissue processing on the content and integrity of nucleic acids. Am J Pathol 2002, 161:1961-1971.
28. Urieli-Shoval S, Meek R L, Hanson R H, Ferguson M, Gordon D, Benditt E P: Preservation of RNA for in situ hybridization: Carnoy's versus formaldehyde fixation. J Histochem Cytochem 1992, 40:1879-1885.
29. Carbone A, Cilia A M, Gloghini A, Capello D, Perin T, Bontempo D, Canzonieri V, Tirelli U, Volpe R, Gaidano G: Primary effusion lymphoma cell lines harbouring human herpesvirus type-8. Leuk Lymphoma 2000, 36:447-456.
30. Carbone A, Gloghini A, Vaccher E, Zagonel V, Pastore C, Dalla Palma P, Branza F, Saglio G, Volpe R, Tirelli U, Gaidano G: Kaposi's sarcoma-associated herpesvirus DNA sequences in AIDS-related and AIDS-unrelated lymphomatous effusions. Br J Haematol 1996, 94:533-543.
31. Mehra M: RNA isolation from cells and tissue. Krieg PAA eds. Laboratory Guide to RNA: Isolation, Analysis and Synthesis. 1996:1-20 Wiley-Liss New York.
32. Chomczynski P, Sacchi N: Single-step method of RNA isolation by acid guanidinium thiocyanate-phenol-chloroform extraction. Anal Biochem 1987, 162:156-159.
33. Jackson D P, Lewis F A, Taylor G R, Boylston A W, Quirke P: Tissue extraction of DNA and RNA and analysis by the polymerase chain reaction. J Clin Pathol 1990, 43:499-504.
34. Krafft A E, Duncan B W, Bijwaard K E, Taubenberger J K, Lichy J H: Optimization of the isolation and amplification of RNA from formalin-fixed, paraffin-embedded tissue: the Armed Forces Institute of Pathology experience and literature review. Mol Diagn 1997, 2:217-230.
35. Schneider, S., Uchida, K., Salonga, D., Yochim, J.-M., Danenberg, K. D. and Danenberg, P. V. Quantitative determination of p16 gene pression by RT-PCR. Methods Mol. Biol., 281:91-103, 2000.
36. Heid, C. A., Stevens, J., Livak, K. J. and Williams, P. M. Real time quantitative PCR. Genome Res., 6: 986-994, 1996.
37. Gibson, U. E., Heid, C. A. and Williams, P. M. A novel method for real time quantitative RT-PCR. Genome Res., 6: 995-1001, 1996.
38. Bijwaard K E, Fetsch J F, Przygodzki R, Taubenberger J K, Lichy J H. Detection of SYT-SSX fusion transcripts in archival synovial sarcomas by real-time reverse transcriptase-polymerase chain reaction. J Mol. Diagn. 2002; 4:59-64.

What is claimed is:

1. A method for the isolation of long fragment RNA from a fixed tissue sample comprising the following:
a) heating the fixed tissue sample in an extraction solution to a temperature in the range of about 44 to about 62° C. for a time period of 3 hours or more, wherein the extraction solution comprises a chelator at a concentration of about 0.1 mM to about 20 mM, and proteinase K; and
b) removing DNA contamination; and
c) isolating said RNA from said extraction solution.

2. The method of claim 1 wherein the heating is selected from the group consisting of a temperature range from about 45 to about 60° C., from about 48 to about 58° C., from about 48 to about 55° C., from about 48 to 52° C., and about 50° C.

3. The method of claim 1 wherein the heating is from about 50-56° C.

4. The method of claim 1 wherein the time period is greater than 4 hours.

5. The method of claim 4 wherein the time period is greater than 8 hours.

6. The method of claim 5 wherein the time period is greater than 12 hours.

7. The method of claim 6 wherein the time period is greater than 14 hours.

8. The method of claim 7 wherein the time period is about 16 hours.

9. The method of claim 3 wherein the time period is about 16 hours.

10. The method of claim 1 wherein the chelator is selected from the group consisting of EDTA, EGTA, citrates, citric acids, salicylic acid, salts of salicylic acids, phthalic acids, 2,4-pentanedines, histidines, histidinol dihydrochlorides, 8-hydroxyquinolines, 8-hydroxyquinoline, citrates and o-hydroxyquinones.

11. The method of claim 1 wherein the chelator is EDTA.

12. The method of claim 1 wherein the chelator is sodium citrate.

13. The method of claim 1 wherein the chelator is present at a concentration of about 0.6 mM to about 5.0 mM.

14. The method of claim 1 wherein the chelator is present at a concentration of about 0.6 mM to about 3.6 mM.

15. The method of claim 1 wherein the chelator is present at a concentration of 3.6 mM.

16. The method of claim 11 wherein EDTA is present at about 3.6 mM.

17. The method of claim 12 wherein sodium citrate is present at about 0.6 mM to about 3.6 mM.

18. The method of claim 1 wherein removing DNA contamination is performed with a first and a second phenol extraction wherein the second phenol extraction comprises a chaotropic agent

19. The method of claim 1 wherein the chaotropic agent is selected from the group consisting of urea, guanidinium isothiocyanate, sodium thiocyanate (NaSCN), Guanidine HCl, guanidinium chloride, guanidinium thiocyanate, lithium tetrachloroacetate, sodium perchlorate, rubidium tetrachloroacetate, potassium iodide and cesium trifluoroacetate.

20. The method of claim 1 wherein the chaotropic agent is guanidinium isothiocyanate.

21. The method of claim 1 wherein the fixed sample is a formalin-fixed paraffin embedded tissue sample.

22. The method of claim 21 wherein the fixed formalin-fixed paraffin embedded tissue sample is 5 years old or younger.

23. The method of claim 1 wherein no DNAse is employed.

24. A method for the isolation of long fragment RNA from comprising the following:
a) heating a fixed tissue sample in an extraction solution to a temperature in the range of about 50° C. to about 56° C. for a time period of about 16 hours, and
b) performing at least a first and a second phenol extraction wherein the second phenol extraction comprises a chaotropic agent, and isolating said RNA from said extraction solution.

25. A method for the isolation of long fragment RNA from comprising the following:
a) heating a formalin-fixed paraffin embedded tissue sample in an extraction solution to a temperature in the range of about 45 to about 62° C. for a time period of 3 hours or more; wherein the extraction solution comprises a chelator at a concentration of 2.5 mM to about 5.0 mM and proteinase K at a concentration of 12.5 μg proteinase K/mL;
b) performing at least a first and a second phenol extraction wherein the second phenol extraction comprises a chaotropic agent, and isolating said RNA from said extraction solution.

26. A method for the extraction of long fragment RNA from formalin-fixed paraffin embedded tissue comprising the following:
a) heating a fixed paraffin-embedded tissue sample in an extraction solution comprising EDTA or sodium citrate at a concentration of about 3.6 mM and proteinase K at a concentration of 12.5 μg proteinase K/mL to a temperature of about 50° C.-56° C. for a time period of about 16; and
b) performing at least a first and a second phenol extraction wherein the second phenol extraction comprises a chaotropic agent, and isolating said RNA from said extraction solution.

27. The method of claim 1, in which the long fragment RNA is longer than 200 nucleotides in length.

28. The method of claim 1, in which the long fragment RNA is 300 nucleotides or longer.

29. The method claim 1, wherein the extraction method co-isolates less than 10% DNA.

30. Long fragment RNA isolated by the method of claim 1.

31. cDNA generated from the long fragment RNA of claim 30.

32. Use of the RNA of claim 30 in gene expression analysis.

33. A method for determining the level of a target gene expression in a fixed paraffin embedded tissue sample comprising:
(a) isolating long fragment RNA from the tissue sample by the method of claim 1;
(b) subjecting the mRNA to amplification using a pair of oligonucleotide primers capable of amplifying a region of the target gene, to obtain amplified mRNA; and
(c) determining the quantity of the target gene mRNA relative to the quantity of an internal control gene's mRNA.

34. The method of claim 33 wherein the target gene is ERCC1, TS, DPD, Her2neu, Gst-pi, RRM1, or Kras.
